SEQUENCE LISTING (62 sequences)

SEQ ID NO: 1 - protein, 133 aa, Homo sapiens
  misc_feature (1)..(133): Wild-type Human pre-cursor Norrin (133 a.a.)
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 2 - protein, 133 aa, Homo sapiens
  misc_feature (93)..(93): Xaa is any amino acid other than C and A
MRKHVLAASFSMLSLLVIMGDTDSKTDSSFIMDSDPRRCMRHHYVDSISHPLYKCSSKMVLLARCEGHCSQASRSEPLVSFSTVLKQPFRSSXHCCRPQTSKLKALRLRCSGGMRLTATYRYILSCHCEECNS

SEQ ID NO: 3 - protein, 133 aa, Homo sapiens
  misc_feature (95)..(95): Xaa is any amino acid other than C and A
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHXCRPQTSKLKALRLRCSGGMRLTATYRYILSCHCEECNS

SEQ ID NO: 4 - protein, 133 aa, Homo sapiens
  misc_feature (131)..(131): Xaa is any amino acid other than C and A
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEEXNS

SEQ ID NO: 5 - protein, 133 aa, Homo sapiens
  misc_feature (89)..(89): Xaa is any amino acid other than F  and  L
MRKHVLAASFSMLSLLVIMGDTDSKTDSSFIMDSDPRRCMRHHYVDSISHPLYKCSSKMVLLARCEGHCSQASRSEPLVSFSTVLKQPXRSSCHCCRPQTSKLKALRLRCSGGMRLTATYRYILSCHCEECNS

SEQ ID NO: 6 - protein, 133 aa, Homo sapiens
  misc_feature: Xaa is any amino acid other than I
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYXLSCHCEECNS

SEQ ID NO: 7 - protein, 133 aa, Homo sapiens
  misc_feature (41)..(41): Xaa is any amino acid other than R, K, T, and S
MRKHVLAASFSMLSLLVIMGDTDSKTDSSFIMDSDPRRCMXHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 8 - protein, 133 aa, Homo sapiens
  misc_feature (43)..(43): Xaa is any amino acid other than H, R and Q
MRKHVLAASFSMLSLLVIMGDTDSKTDSSFIMDSDPRRCMRHXYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 9 - protein, 133 aa, Homo sapiens
  misc_feature (44)..(44): Xaa is any amino acid other than Y and C
MRKHVLAASFSMLSLLVIMGDTDSKTDSSFIMDSDPRRCMRHHXVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 10 - protein, 133 aa, Homo sapiens
  misc_feature (45)..(45): Xaa is any amino acid other than V, M and E
MRKHVLAASFSMLSLLVIMGDTDSKTDSSFIMDSDPRRCMRHHYXDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 11 - protein, 133 aa, Homo sapiens
  misc_feature (43)..(43): Xaa is any amino acid other than H, R and Q
MRKHVLAASFSMLSLLVIMGDTDSKTDSSFIMDSDPRRCMRHXXVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 12 - protein, 133 aa, Homo sapiens
  misc_feature (43)..(43): Xaa is any amino acid other than H, R and Q
MRKHVLAASFSMLSLLVIMGDTDSKTDSSFIMDSDPRRCMRHXYXDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 13 - protein, 133 aa, Homo sapiens
  misc_feature (44)..(44): Xaa is any amino acid other than Y and C
MRKHVLAASFSMLSLLVIMGDTDSKTDSSFIMDSDPRRCMRHHXXDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 14 - protein, 133 aa, Homo sapiens
  misc_feature (43)..(43): Xaa is any amino acid other than H, R and Q
MRKHVLAASFSMLSLLVIMGDTDSKTDSSFIMDSDPRRCMRHXXXDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 15 - protein, 133 aa, Homo sapiens
  misc_feature (59)..(59): Xaa is any amino acid other than M
MRKHVLAASFSMLSLLVIMGDTDSKTDSSFIMDSDPRRCMRHHYVDSISHPLYKCSSKXVLLARCEGHCSQASRSEPLVSFSTVLKQPFRSSCHCCRPQTSKLKALRLRCSGGMRLTATYRYILSCHCEECNS

SEQ ID NO: 16 - protein, 133 aa, Homo sapiens
  misc_feature (60)..(60): Xaa is any amino acid other than V and E
MRKHVLAASFSMLSLLVIMGDTDSKTDSSFIMDSDPRRCMRHHYVDSISHPLYKCSSKMXLLARCEGHCSQASRSEPLVSFSTVLKQPFRSSCHCCRPQTSKLKALRLRCSGGMRLTATYRYILSCHCEECNS

SEQ ID NO: 17 - protein, 133 aa, Homo sapiens
  misc_feature (61)..(61): Xaa is any amino acid other than L, F, I and P
MRKHVLAASFSMLSLLVIMGDTDSKTDSSFIMDSDPRRCMRHHYVDSISHPLYKCSSKMVXLARCEGHCSQASRSEPLVSFSTVLKQPFRSSCHCCRPQTSKLKALRLRCSGGMRLTATYRYILSCHCEECNS

SEQ ID NO: 18 - protein, 133 aa, Homo sapiens
  misc_feature (59)..(59): Xaa is any amino acid other than M
MRKHVLAASFSMLSLLVIMGDTDSKTDSSFIMDSDPRRCMRHHYVDSISHPLYKCSSKXXLLARCEGHCSQASRSEPLVSFSTVLKQPFRSSCHCCRPQTSKLKALRLRCSGGMRLTATYRYILSCHCEECNS

SEQ ID NO: 19 - protein, 133 aa, Homo sapiens
  misc_feature (59)..(59): Xaa is any amino acid other than M
MRKHVLAASFSMLSLLVIMGDTDSKTDSSFIMDSDPRRCMRHHYVDSISHPLYKCSSKXVXLARCEGHCSQASRSEPLVSFSTVLKQPFRSSCHCCRPQTSKLKALRLRCSGGMRLTATYRYILSCHCEECNS

SEQ ID NO: 20 - protein, 133 aa, Homo sapiens
  misc_feature (60)..(60): Xaa is any amino acid other than V and E
MRKHVLAASFSMLSLLVIMGDTDSKTDSSFIMDSDPRRCMRHHYVDSISHPLYKCSSKMXXLARCEGHCSQASRSEPLVSFSTVLKQPFRSSCHCCRPQTSKLKALRLRCSGGMRLTATYRYILSCHCEECNS

SEQ ID NO: 21 - protein, 133 aa, Homo sapiens
  misc_feature (120)..(120): Xaa is any amino acid other than Y and C
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATXRYILSCHCEECNS

SEQ ID NO: 22 - protein, 133 aa, Homo sapiens
  misc_feature (121)..(121): Xaa is any amino acid other than R, G, W, Q and L
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYXYILSCHCEECNS

SEQ ID NO: 23 - protein, 133 aa, Homo sapiens
  misc_feature (122)..(122): Xaa is any amino acid other than Y
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRXILSCHCEECNS

SEQ ID NO: 24 - protein, 133 aa, Homo sapiens
  misc_feature (120)..(120): Xaa is any amino acid other than Y and C
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATXXYILSCHCEECNS

SEQ ID NO: 25 - protein, 133 aa, Homo sapiens
  misc_feature (120)..(120): Xaa is any amino acid other than Y and C
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATXRXILSCHCEECNS

SEQ ID NO: 26 - protein, 133 aa, Homo sapiens
  misc_feature (121)..(121): Xaa is any amino acid other than R, G, W, Q and L
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYXXILSCHCEECNS

SEQ ID NO: 27 - protein, 133 aa, Homo sapiens
  misc_feature (52)..(52): Xaa is any amino acid other than L
MRKHVLAASFSMLSLLVIMGDTDSKTDSSFIMDSDPRRCMRHHYVDSISHPXYKCSSKMVLLARCEGHCSQASRSEPLVSFSTVLKQPFRSSCHCCRPQTSKLKALRLRCSGGMRLTATYRYILSCHCEECNS

SEQ ID NO: 28 - protein, 133 aa, Homo sapiens
  misc_feature (53)..(53): Xaa is any amino acid other than Y
MRKHVLAASFSMLSLLVIMGDTDSKTDSSFIMDSDPRRCMRHHYVDSISHPLXKCSSKMVLLARCEGHCSQASRSEPLVSFSTVLKQPFRSSCHCCRPQTSKLKALRLRCSGGMRLTATYRYILSCHCEECNS

SEQ ID NO: 29 - protein, 133 aa, Homo sapiens
  misc_feature (54)..(54): Xaa is any amino acid other than K and N
MRKHVLAASFSMLSLLVIMGDTDSKTDSSFIMDSDPRRCMRHHYVDSISHPLYXCSSKMVLLARCEGHCSQASRSEPLVSFSTVLKQPFRSSCHCCRPQTSKLKALRLRCSGGMRLTATYRYILSCHCEECNS

SEQ ID NO: 30 - protein, 133 aa, Homo sapiens
  misc_feature (52)..(52): Xaa is any amino acid other than L
MRKHVLAASFSMLSLLVIMGDTDSKTDSSFIMDSDPRRCMRHHYVDSISHPXXKCSSKMVLLARCEGHCSQASRSEPLVSFSTVLKQPFRSSCHCCRPQTSKLKALRLRCSGGMRLTATYRYILSCHCEECNS

SEQ ID NO: 31 - protein, 133 aa, Homo sapiens
  misc_feature (52)..(52): Xaa is any amino acid other than L
MRKHVLAASFSMLSLLVIMGDTDSKTDSSFIMDSDPRRCMRHHYVDSISHPXYXCSSKMVLLARCEGHCSQASRSEPLVSFSTVLKQPFRSSCHCCRPQTSKLKALRLRCSGGMRLTATYRYILSCHCEECNS

SEQ ID NO: 32 - protein, 133 aa, Homo sapiens
  misc_feature (53)..(53): Xaa is any amino acid other than Y
MRKHVLAASFSMLSLLVIMGDTDSKTDSSFIMDSDPRRCMRHHYVDSISHPLXXCSSKMVLLARCEGHCSQASRSEPLVSFSTVLKQPFRSSCHCCRPQTSKLKALRLRCSGGMRLTATYRYILSCHCEECNS

SEQ ID NO: 33 - protein, 133 aa, Homo sapiens
  misc_feature (107)..(107): Xaa is any amino acid other than R
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALXLRCSGGMRLTATYRYILSCHCEECNS

SEQ ID NO: 34 - protein, 133 aa, Homo sapiens
  misc_feature (109)..(109): Xaa is any amino acid other than R
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLXCSGGMRLTATYRYILSCHCEECNS

SEQ ID NO: 35 - protein, 133 aa, Homo sapiens
  misc_feature (115)..(115): Xaa is any amino acid other than R and L
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMXLTATYRYILSCHCEECNS

SEQ ID NO: 36 - protein, 133 aa, Homo sapiens
  misc_feature (109)..(109): Xaa is any amino acid other than R
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLXCSGGMXLTATYRYILSCHCEECNS

SEQ ID NO: 37 - protein, 133 aa, Homo sapiens
  misc_feature (54)..(54): Xaa is any amino acid other than K and N
MRKHVLAASFSMLSLLVIMGDTDSKTDSSFIMDSDPRRCMRHHYVDSISHPLYXCSSKMVLLARCEGHCSQASRSEPLVSFSTVLKQPFRSSCHCCRPQTSKLKALRLXCSGGMRLTATYRYILSCHCEECNS

SEQ ID NO: 38 - protein, 133 aa, Homo sapiens
  misc_feature (55)..(55): Xaa is any amino acid other than C, R, and F
MRKHVLAASFSMLSLLVIMGDTDSKTDSSFIMDSDPRRCMRHHYVDSISHPLYKXSSKMVLLARCEGHCSQASRSEPLVSFSTVLKQPFRSSCHCCRPQTSKLKALRLRXSGGMRLTATYRYILSCHCEECNS

SEQ ID NO: 39 - DNA, 327 bp, Homo sapiens
  misc_feature (1)..(327): Mature Human Norrin DNA sequence of SEQ ID NO41
aaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcc

SEQ ID NO: 40 - protein, 483 aa, Artificial Sequence
  Recombinant MBP-Norrin protein sequence
MAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNAAAEFKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 41 - protein, 109 aa, Homo sapiens
  misc_feature (1)..(109): Mature Human Norrin protein sequence
KTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 42 - protein, 374 aa, Escherichia coli
  misc_feature (1)..(374): MBP protein sequence
MAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNAAAEF

SEQ ID NO: 43 - DNA, 1452 bp, Homo sapiens
  misc_feature (1)..(1452): MBP-Human Norrin fusion DNA sequence
atggcaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaacgcggcggccgaattcaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctga

SEQ ID NO: 44 - DNA, 1122 bp, Homo sapiens
  misc_feature (1)..(1122): MBP-DNA sequence
atggcaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaacgcggcggccgaattc

SEQ ID NO: 45 - protein, 109 aa, Homo sapiens; identical to SEQ ID NO: 41

SEQ ID NO: 46 - protein, 109 aa, Canis lupus
KTDSSFMMDSDPLRCMRHHYVDSISHPLYKCSSKMVLLARCEGHCSQASRSEPLVSFSTVLKQPFRSSCHCCRPQTSKLKALRLRCSGGMRLTATYRYILSCHCEECSS

SEQ ID NO: 47 - protein, 107 aa, Mus musculus
KTDSSFLMDSQRCMRHHYVDSISHPLYKCSSKMVLLARCEGHCSQASRSEPLVSFSTVLKQPFRSSCHCCRPQTSKLKALRLRCSGGMRLTATYRYILSCHCEECSS

SEQ ID NO: 48 - protein, 109 aa, Monodelphis domestica
KMDSAFLMDSDPGRCMRHHYVDSISHPLYKCSSKMVLLARCEGHCSQPSRSEPLVSFSTVLKQPFRSTCHCCRPQTSKLKALRLRCSGGMRLTATYRYILSCHCEECSS

SEQ ID NO: 49 - protein, 109 aa, Gallus gallus
KPDSSFMIDNDPSRCMRHHYVDSISHPLYKCSSKMVLLARCEGRCSQTSRSEPMVSFSTVLKQPFRSTCHCCRPQTSKLKAMRLRCSGGMRLTATYRYILSCHCEECNS

SEQ ID NO: 50 - protein, 109 aa, Taeniopygia guttata
KTEGSFLMDSDPSRCMRHHYVDSISHPLYKCSSKMVLLARCEGRCSQTSRSEPMVSFSTVLKQPFRSTCHCCRPQTSKLKAMRLRCSGGMRLTATYRYILSCHCEECNS

SEQ ID NO: 51 - protein, 110 aa, Xenopus laevis
DKKGKAFLAESDPDRCMRHHYVDSINHPQHKCTAKMVLLARCEGRCSQTSRSDPLVSFSTVLKQPFRSTCHCCRPQTSKLKAIRLRCSGGRRMTATYRYILSCHCEECNS

SEQ ID NO: 52 - protein, 108 aa, Danio rerio
KAESGHLGDNDPDRCMRHHFVETITHPIYKCNSKMVLLARCEGHCSHTSRSDPLISFSSVLKQPFKNTCFCCRPHTSKLKAVRLRCSGGTRITATYRYILACSCEECS

SEQ ID NO: 53 - protein, 109 aa, Ornithorhynchus anatinus
KMDRTVRTDADPGQCMRHHYVDSISHPLYKCSSKMVLLARCEGRCSQSSRSEPLVSFSAVLKQPFRSTCYCCRPQTSKLKALRLRCSGGMRLTATYRYILSCHCEQCSS

SEQ ID NO: 54 - protein, 104 aa, Homo sapiens
  misc_feature (1)..(104): von Willebrand factor
KIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 55 - protein, 102 aa, Homo sapiens
  misc_feature (1)..(102): Mucin2
CCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLP

SEQ ID NO: 56 - protein, 97 aa, Sus scrofa
  misc_feature (1)..(97): Mucin-pig
CCYTCKSSCKPSPVNVTVRYNGCTIKVEMARCVGECKKTVTYDYDIFQLKNSCLCCQEEDYEERDIVLDCPDGSTLLPYRYRHITACSCLDDPCQQS

SEQ ID NO: 57 - protein, 112 aa, Homo sapiens
  misc_feature (1)..(112): TGF-beta1
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 58 - protein, 112 aa, Homo sapiens [sequence omitted]

SEQ ID NO: 59 - protein, 112 aa, Homo sapiens
  misc_feature (1)..(112): TGF-beta2
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 60 - protein, 133 aa, Homo sapiens
  misc_feature (55)..(55): X is any other amino acid other than C, R, and F
MRKHVLAASFSMLSLLVIMGDTDSKTDSSFIMDSDPRRCMRHHYVDSISHPLYKXSSKMVLLARCEGHCSQASRSEPLVSFSTVLKQPFRSSCHCCRPQTSKLKALRLRCSGGMRLTATYRYILSCHCEECNS

SEQ ID NO: 61 - protein, 133 aa, Homo sapiens
  misc_feature (65)..(65): X is any other amino acid other than C, W, and Y
MRKHVLAASFSMLSLLVIMGDTDSKTDSSFIMDSDPRRCMRHHYVDSISHPLYKCSSKMVLLARXEGHCSQASRSEPLVSFSTVLKQPFRSSCHCCRPQTSKLKALRLRCSGGMRLTATYRYILSCHCEECNS

SEQ ID NO: 62 - protein, 133 aa, Homo sapiens
  misc_feature (110)..(110): X is any other amino acid other than C, R, G, and S
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRXSGGMRLTATYRYILSCHCEECNS

(19) United States
(12) Patent Application Publication
(10) Pub. No.: US 2015/0376252 A1
(43) Pub. Date: Dec. 31, 2015

(54) Norrin Mutant Polypeptides, Methods of Making and Uses Thereof

(71) Applicant: VAN ANDEL RESEARCH INSTITUTE, Grand Rapids, MI (US)

(72) Inventors: Huaqiang Eric Xu, Grand Rapids, MI (US); Jiyuan Ke, Grand Rapids, MI (US)

(73) Assignee: VAN ANDEL RESEARCH INSTITUTE, Grand Rapids, MI (US)

(21) Appl. No.: 14/769,166

(22) PCT Filed: Feb. 20, 2014
(86) PCT No.: PCT/US14/17493

§ 371 Date: Aug. 20, 2015

Related U.S. Application Data
(60) Provisional application No. 61/767,569, filed on Feb. 21, 2013.

(51) Int. Cl. C07K 14/475; A61K 45/06

(52) U.S. Cl. CPC C07K 14/475; A61K 45/06; C07K 2319/24

(57) ABSTRACT

The present invention relates to Norrin mutant polypeptides that inhibit or reduce angiogenesis in various tissues. Methods for synthesizing recombinant Norrin and Norrin mutant polypeptides are provided. Methods of inhibiting or reducing aberrant angiogenesis comprise contacting a tissue undergoing aberrant angiogenesis with a composition comprising an isolated Norrin C mutant polypeptide.

### CROSS-REFERENCE TO RELATED APPLICATIONS

This PCT application claims the benefit of U.S. Provisional Application Ser. No. 61/767,569, filed Feb. 21, 2013. The disclosure of this document is hereby incorporated by reference in its entirety.

### INCORPORATION BY REFERENCE OF MATERIAL SUBMITTED ELECTRONICALLY

Incorporated by reference in its entirety is a computer-readable sequence listing submitted concurrently herewith and identified as follows: One 82 KB ASCII (Text) file named “232119-349809_Sequence_Listing_ST25.txt,” created on Feb. 20, 2014, at 7:20 pm.

### TECHNICAL FIELD

The present invention relates generally to isolated Norrin wild-type and mutant polypeptides. The present invention also relates to methods of making wild-type and mutant Norrin polypeptides and uses of these Norrin polypeptides to treat various diseases and disorders associated with aberrant angiogenesis.

### BACKGROUND

Wnt signaling plays essential roles in animal embryogenesis, tissue homeostasis, and numerous diseases (Huelsken and Behrens, 2002; Logan and Nusse, 2004). The canonical Wnt signaling pathway is mediated by the direct binding of Wnt ligands to the Frizzled (Fz) family of receptors and the Wnt co-receptor, low density lipoprotein receptor-related protein (Lrp) 5/6, which typically results in activation of the β-catenin/TCF transcriptional pathway (He et al., 2004; Wodarz and Nusse, 1998). Norrin is an atypical Wnt ligand, which can activate the canonical β-catenin signaling through its specific binding to Fz4 receptor (Xu et al., 2004). Norrin is encoded by the Norrie Disease Protein (NDP) gene, which is associated with Norrie disease (ND). Positional cloning identified mutations in the X-linked NPD gene as principal cause of ND, a severe, but rare, retinal hypovascularization disease that generally leads to blindness, mental retardation, and deafness, as well as a cause for the related, milder disorder, Familial Exudative Vitreoretinopathy (FEVR) (Berger, 1998; Berger et al., 1992; Chen et al., 1992; Warburg, 1966). To date over 100 different disease-causing NPD mutations have been mapped to the Norrin gene (Ye et al., 2010).

Norrin plays important roles in angiogenesis not only for eye development, but also for the development of ear, brain and the female reproductive system (Berger et al., 1996; Chen et al.,) 1993; Luhmann et al., 2005a; Luhmann et al., 2005b; Ohlmann et al., 2005; Rehm et al., 2002; Xu et al., 2004). Norrin also has pronounced neuroprotective properties for retinal neurons (Seitz et al., 2010). Recently, it was shown that Norrin is also required for maintenance of the integrity of blood brain barrier and blood retina barrier, loss of which are involved in a wide range of diseases including age-related macular degeneration and diabetic macular edema (Wang et al., 2012). Retinal hypervascularization diseases, such as diabetic retinopathy, age-related macular degeneration (AMD), and retinopathy of prematurity (ROP) are leading causes of vision impairment and blindness, that collectively affect about 20 million patients in the United States alone.

Like Wnt ligands, Norrin specifically binds with high affinity to the N-terminal extracellular Cysteine Rich Domain (CRD) of Fz4 and activates the β-catenin signaling pathway (Xu et al., 2004). Mutations in Fz4 also cause FEVR, suggesting they function in the same pathway and underscoring the importance of the Norrin/Fz4 mediated β-catenin pathway for eye vascularization (Xu et al., 2004; Ye et al., 2009). Importantly, mutations in the gene encoding Lrp5 also cause the ocular disorder FEVR (Berger, 1998; He et al., 2004; Robitaille et al., 2002; Xu et al., 2004), suggesting Lrp5 may function in the same pathway as Norrin and Fz4. Moreover, cotransfection with Lrp5 or its close homolog Lrp6 greatly potentiates Norrin-mediated Fz4 activation (Xu et al., 2004). These results suggest that Lrp5/6 may function as Norrin co-receptors, analogous to their role as Wnt co-receptors. However, the mechanism of how Lrp5/6 participates in Norrin/Fz4 signaling remains puzzling as Norrin, in contrast to Wnt ligands, did not show a direct interaction with Lrp5/6 in cell-based binding assays (Junge et al., 2009; Xu et al., 2004). Finally, a tetraspanin family protein, TSPAN12, was identified as additional factor specific for Norrin-induced, but not Wnt-induced 3-catenin signaling. TSPAN12 is thought to have chaperone activity to further stabilize Norrin-Fz4 signaling complexes (Junge et al., 2009).

Norrin belongs to the cysteine-knot growth factor superfamily and shows weak homology (sequence identity ≦17%) to members of the Transforming Growth Factor-β(TGF-β) family, but has no sequence similarity to the canonical Wnt ligands. No structural information is available for any close homolog to Norrin in part due to the difficulties in obtaining pure proteins for structural and functional studies.

Angiogenesis is the process of blood vessel growth from pre-existing vasculatures. In recent years, angiogenesis has been elucidated as an important physiological phenomenon in proliferation and metastasis of various progressive solid cancers. Angiogenesis proceeds through multiple steps including, for example, 1) stimulation by vascular endothelial growth factor secreted from tumor cells; 2) disengagement of peritheliocyte or decomposition or digestion of extracellular matrix such as basal membrane; 3) migration and proliferation of vascular endothelial cells; 4) formation of tubules by the endothelial cells, formation of basal membrane, and maturation of blood vessel. In tumorous angiogenesis, the new vessels generated have the role of supplying oxygen and nutrient to tumors to accelerate their growth and serving as a route for infiltration and metastasis of tumor cells to other cells.

Age-related macular degeneration (AMD) is an angiogenesis-mediated ocular disorder in humans and is the leading cause of visual loss in individuals over age 55 (Ferris et al. 1984 Arch. Opthalmol. 102:1640-1642). There are two major clinical types of AMD: non-exudative (dry) type and exudative (wet) type. One of the pathological complications of age-related macular degeneration is choroidal angiogenesis or choroidal neovascularization (CNV). CNV is responsible for the sudden and disabling loss of central vision.

CNV is a complex biological process and the pathogenesis of new choroidal vessel formation is not fully understood. Several factors such as inflammation, ischemia, and local production of angiogenic factors are thought to be important in the pathogenesis of CNV.

Despite extensive research in the field of angiogenesis inhibition, particularly for ocular diseases and disorders, there remains a need to identify targets and develop novel agents that are capable of inhibiting angiogenesis that may also complement or enhance the activity of existing anti-angiogenic therapies.

### SUMMARY

The present invention relates in part to the finding that Norrin, an atypical Wnt ligand induces formation of a ternary complex of Fz4 with Lrp5/6 by binding to their respective extracellular domains. The ternary complex is then able to exert further activation of the β-catenin/LRP transcriptional pathway and induce angiogenesis. In view of the sensitively orchestrated binding and conformational requirements of each of the complex′ participants, disruption to the structure of Norrin leads to inhibition of Norrin-Fz4 and/or Lrp5/6 mediated signaling. The inhibition of Norrin-Fz4 mediated signaling is believed to further effectuate an anti-angiogenic response in tissue subjected to angiogenic stimuli.

In one aspect of the present invention, Norrin mutant polypeptides are provided. The Norrin mutant polypeptides are believed to inhibit, reduce, or attenuate the activation of canonical Wnt signaling previously shown to be an important mediator of vascular development.

In accordance with the present invention, in one aspect, the invention provides an isolated Norrin mutant polypeptide, the Norrin mutant polypeptide comprising the amino acid sequence of SEQ ID NO: 1, the polypeptide comprising the amino acid sequence of SEQ ID NO: 1, the sequence having one or more amino acid substitutions at positions: 93, 95, 131, 89, 123, 41, 43, 44, 45, 59, 60, 61, 120, 121, 122, 52, 53, 54, 107, 109, 115, 55, and 110 relative to SEQ ID NO: 1.

In accordance with the present invention, in another related aspect the invention provides an isolated Norrin mutant polypeptide wherein the polypeptide has two to seven, two to five, two to four, one to three, three, two, or one amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1. In accordance with the present invention, in one aspect, the invention provides an isolated Norrin mutant polypeptide, wherein the polypeptide has an amino acid sequence having one or more amino acid substitutions at positions: 131, 59, 122, 52, 53, 93, 107, and 109 of SEQ ID NO:1. In some embodiments, the isolated Norrin mutant polypeptide has one or more amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another embodiment, the isolated Norrin mutant polypeptide can have two or more amino acid substitutions selected from: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, for example, a substitution: C93A, C131A, M59A, Y122A, L52A, Y53A, and R107E, or for example, M59A, Y122A, L52A, Y53A, and R107E. In one example, the Norrin mutant polypeptide an amino acid sequence having one to seven amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E.

In another aspect, the present invention provides an isolated Norrin mutant polypeptide wherein the polypeptide comprises an amino acid sequence of SEQ ID NOs: 2-38 and 60-62, for example, an amino acid sequence of SEQ ID NO:2, 4, 15, 23, 26, 27 or 33. In accordance with the present invention, in another aspect, the invention provides an isolated Norrin mutant polypeptide, wherein the polypeptide has an amino acid sequence having one or more amino acid substitutions as shown in Table 1. In one such example, the polypeptide comprising the amino acid sequence of SEQ ID NO: 1, and having one or more amino acid substitutions at positions 93, 131, 59, 122, 52, 53 107, and 109. In one such example, the isolated Norrin mutant polypeptide has an amino acid sequence having one or more amino acid substitutions relative to SEQ ID NO:1 selected from the group consisting of: C93A, C95A, C131A, F89R, R41E, H43A, Y44A, V45A, M59A, L61A, Y120A, R121A, Y122A, L52A, Y53A, K54A, R107E, R109E, and R115E. For example, in one embodiment, the isolated Norrin mutant polypeptide has an amino acid sequence having one, or two or more amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another example, the isolated Norrin mutant polypeptide has an amino acid sequence having two to seven amino acid substitutions selected from: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or two to five amino acid substitutions relative to SEQ ID NO:1, selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or one, two or three amino acid substitutions relative to SEQ ID NO:1 selected from C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E.

In accordance with the present invention, in one aspect, the invention provides an isolated Norrin mutant polypeptide, wherein the polypeptide has an amino acid sequence having one to seven amino acid substitutions relative to SEQ ID NO:1 comprising: C93A, C95A, C131A, F89R, R41E, H43A, Y44A, V45A, M59A, L61A, Y120A, R121A, Y122A, L52A, Y53A, K54A, R107E, R109E, and R115E. In various aspects, an exemplary isolated Norrin mutant polypeptide has an amino acid substitution as defined above, wherein the amino acid substitution or substitutions is or are, conservative amino acid substitutions.

In accordance with the present invention, in another aspect, the invention provides an isolated Norrin mutant polypeptide, the polypeptide comprising the amino acid sequence of SEQ ID NO: 1, wherein the amino acid sequence of SEQ ID NO:1 has one or more, or one to seven, or one to five, or one, two or three amino acid substitutions at positions 131, 59, 122, 52, 53, 93, 107, and 109 relative to SEQ ID NO: 1.

In accordance with the present invention, in one aspect, the invention provides an isolated fusion protein comprising a Norrin mutant polypeptide of the present invention fused to a maltose binding protein.

In another aspect, the present invention provides a method for the purification and isolation of Norrin and Norrin mutant polypeptides. In some embodiments, the method for purifying the polypeptides includes:

a. providing a nucleic acid comprising a nucleic acid sequence encoding a bacterial maltose binding protein (MBP) operatively fused to a nucleic acid sequence encoding a Norrin construct;

b. expressing said nucleic acid in a bacterial strain comprising a gor and a trxB genetic mutation;

c. disrupting the integrity of the bacterial cell wall to provide a crude extract;

d. isolating the MBP-Norrin construct from the crude extract using an amylose affinity column; and

e. mixing the isolated MBP-Norrin protein with a shuffling solution comprising arginine, reduced glutathione, oxidized glutathione, and a disulfide bond isomerase. Optionally, step (e) or step (f) comprises removing MBP terminal protein from the MBP-Norrin construct.

Properly folded Norrin and mutant polypeptides thereof produced using the methods described herein can be easily recovered from other components using amylose affinity chromatography. Such purification methods can easily be scaled to produce milligram and gram quantities of a Norrin construct for further investigation. In a related aspect, the present invention also includes a novel recombinant MBP-Norrin fusion protein.

In another aspect, the invention also relates to a method of treating a disease associated with aberrant angiogenesis with a compound of the invention, or a composition or formulation as described herein. In some embodiments, tissue (in vitro or in vivo) that is subject to angiogenic stimuli or has aberrant angiogenesis is treated by administering an anti-angiogenic effective amount of a Norrin mutant polypeptide. In some embodiments, the disease is an ophthalmic disease. In some embodiments, the disease is cancer or metastases of a tumor.

### BRIEF DESCRIPTION OF THE DRAWINGS

FIG. 1A shows an SDS-PAGE electropherogram depicting isolated proteins comprising purified Fz4-FcH6, Fz8-FcH6, and MBP-Norrin proteins separated under non-reducing (lanes 1-3) and reducing conditions (lanes 4-6).

FIG. 1B depicts an elution profile of MBP-Norrin purified on a size exclusion column (120 ml Superdex).

FIG. 1C depicts a graph representing the binding kinetics of MBP-Norrin to Fz4-FcH6, Fz8-FcH6, or human IgG measured by biolayer interferometry.

FIG. 1D(1) depicts a schematic drawing of recombinant biotin Norrin-Fz4Fc.H6 binding using AlphaScreen luminescence proximity screens.

FIG. 1D(2) depicts a bar chart representing binding of recombinant biotin-MBP-Norrin protein to Fz4-FcH6 or Fz8-FcH6.

FIG. 1E depicts a line graph representing saturation binding curve for biotin-MBP-Norrin to Fz4-FcH6 as measured by AlphaScreen assay.

FIG. 1F depicts a bar chart representing luciferase activity from a TCF-mediated luciferase reporter in Fz4-expressing 293STF cells in the presence of bacterially expressed MBP-Norrin.

FIG. 1G depicts a bar chart representing activation of TCF-mediated luciferase activity in cells coexpressing Fz4 and Lrp5 in the presence or absence of MBP-Norrin or Norrin.

FIG. 1H depicts an electropherogram representing MBP-Norrin and MBP-Rhodopsin (control) samples and its separation from aqueous and detergent fractions using an anti-Norrin antibody and anti-MBP antibodies.

FIG. 2A is a graphical representation of the three dimensional structure of a Norrin monomer with four intramolecular disulfide bonds (C39-C96, C65-C126, C69-C128, and C55-C110) shown as stick models. MBP is omitted for clarity.

FIG. 2B is a graphical representation of the crystal structure of a Norrin dimer.

FIG. 2C is a graphical representation of the structure of a Norrin dimer depicting three inter-molecular disulfide bonds (Cys93-Cys95, Cys95-Cys93, Cys131-Cys131).

FIG. 2D is a graphical representation of the crystal structure of a Norrin dimer illustrating the dimer being further stabilized by intermolecular hydrogen bond interactions between β2′ of one monomer with β2 and β4 from the other monomer.

FIG. 2E is a graphical representation of the F89-I123 hydrophobic interaction at the dimeric interface of the Norrin dimer.

FIG. 2F is an electropherogram representing co-immunoprecipitation of MBP-Norrin dimer and binding of two Fz4 CRD domains. Fz4-T4L-v5H6 or T4L-v5H6 were immunoprecipitated with v5-agarose beads. Co-precipitated MBP-Norrin and Fz4-FcH6 were detected by tag-specific antibodies

FIG. 3A is a bar chart showing Fz4 receptors strongly interacted with each other in the absence of any Norrin ligand, producing a BRET signal as strong as that of the positive control.

FIG. 3B is a line chart representing the saturation BRET signals in a sample consisting of a coexpression of a constant amount of Fz4-Rlu with increasing amounts of Fz4-YFP generated a hyperbolic, plateau-reaching signal, indicative of a true interaction, whereas titrating Fz4-Rlu with CCK2R-YFP, an unrelated seven-transmembrane receptor, generated a non-saturating, quasi-linear signal, indicative of random collisions.

FIG. 3C is a bar chart illustrating the effect of three Norrin ligands wild-type, mutant (R41E) and MBP-Norrin construct on the Fz4 BRET signal.

FIG. 3D (1-2) are photomicrographs showing that Fz4 fused to the non-fluorescent complementary halves of YFP, Fz4-YFP-N, and Fz4-YFP-C dimerize and establish a strong YFP fluorescent signal on the cell membrane (arrow heads).

FIG. 3D(3) illustrates the emission spectrum and anisotropy of the signals generated in FIG. 3D(1-2) were appropriate.

FIG. 3E depicts a bar graph indicating that Norrin did not induce higher oligomerization of Fz4 receptors detected by three-component BRET assay as shown by the absence of significant BRET signal from the Rlu-tagged Fz4 co-expressed with Fz4 tagged with YFP-N and YFP-C in the absence or presence of Norrin coexpression. The shaded area represents the background signal as determined using Rlu-tagged Fz4 with CCK2R-YFP.

FIG. 3F depicts line graph representing saturation BRET signals from the Fz4-Rlu/Fz4-YFP pair and the lack of a significant signal from co-expression of the Rlu-tagged Fz4 along with Fz4 tagged with YFP-N and YFP-C.

FIG. 4A depicts a bar chart indicating binding strength of the interactions of biotin-MBP-Norrin with different Lrp6 ECD proteins.

FIG. 4B is a graphical representation of binding of biotinylated MBP-Norrin to Lrp6 ECD proteins (Lrp6BP1-2, Lrp6BP3-4 and Lrp6BP1-4) measured by biolayer interferometry.

FIG. 4C is a line plot of competition data of FIG. 4B using normalized values and calculating the IC₅₀ as 566 nM by nonlinear regression, corresponding to a K_{d} of ≈450 nM.

FIG. 5A is graphical representation of the ribbon diagram of Norrin dimer with the putative residues involved in binding to Fz4 (darker) or Lrp5/6 (lighter) shown as stick models.

FIG. 5B is a bar chart representing the effect of mutations of the above surface residues on Norrin that interfered with Fz4 binding (R41E, H43A/V45A, L61 A and Y120A/Y122A) or Lrp5/6 binding (K54E, R107E, R109E, K54E/R109E, and C55A/C110A) reduced Norrin's signaling activity function determined by AlphaScreen luminescence proximity assay.

FIG. 5C depicts a bar chart representing a competition assay using MBP-Norrin wild-type or mutant proteins to compete with the interaction between biotinylated MBP-Norrin and Fz4-FcH6 protein.

FIG. 5D depicts a bar chart representing a competition assay using MBP-Norrin wild-type or mutant proteins to compete with the interaction between biotinylated DKK1 peptide and H8-Lrp6 BP1-2 protein.

FIG. 6A depicts an electropherogram representing the formation of a Norrin ternary complex with Fz4-CRD and Lrp5 ECD domains using immunoprecipitated complexes.

FIG. 6B depicts a schematic diagram of full length Lrp5 and of the Lrp5 C-terminal truncation (Lrp5NT). Right, a bar graph representing inhibition of Norrin-mediated TCF luciferase reporter activity in the presence of Lrp5-NT in a dominant negative manner. 293STF cells were transfected with Fz4, Fz4+Lrp5, or Fz4+Lrp5-NT in the presence or absence of Norrin expression vector.

FIG. 6C depicts a bar chart representing Norrin/Fz4/Lrp5-mediated β-catenin signaling in the presence and absence Norrin expression vector.

FIG. 6D depicts a bar chart representing static BRET signals in COS-1 cells expressing Rlu- or YFP-tagged Tspan12, and YFP-, YFP-N- and YFP-C-tagged Fz4 receptors.

FIG. 7 depicts a schematic representation of a model for Norrin mediated Fz4 receptor activation.

FIG. 8A depicts a line graph representing the binding avidity between Fz4-FcH6 and MBP-Norrin dimers measured by biolayer interferometry.

FIG. 8B (1&2) is a depiction of a stereo view of the Norrin monomeric structure. Shown are the main chain atoms in stick models with carbon atoms, oxygen atoms, and nitrogen atoms. The 2Fₒ-F_{c} map contoured at 1.0 σ is also shown.

FIG. 9A is a representation of the crystal structure of MBP-Norrin with four dimers shown indicating the MBP-MBP crystal packing interaction site (1) and MBP-Norrin crystal packing interaction sites 2 & 3.

FIG. 9B is a graphical representation of the structure of the MBP-Norrin dimer. On top are two MBP molecules and on the bottom are two Norrin monomers. One MBP-Norrin molecule is related to another molecule by a crystallographic twofold symmetry.

FIG. 10A is a graphical representation of monomeric structures of Norrin and TGF-β3 (PDB code: 1TGJ). The disulfide bonds including the conserved cysteine knot structure (a cluster of three intramolecular disulfide bonds) are shown as stick models. The significant structural differences between them are indicated in four boxes (Boxes 1-4).

FIG. 10B is a graphical representation comparing the dimeric structures of Norrin and TGF-β3 (PDB code: 1TGJ). The intermolecular disulfide bonds are shown as stick models.

FIG. 10C depicts the missense Norrie disease mutations onto the Norrin dimeric structure in graphical form. A Norrin dimer is shown as a Cα backbone ribbon diagram with two monomers shaded in different tones.

FIG. 11A bar chart representing activation of TCF luciferase as a result of Norrin-mediated activation of the downstream TCF luciferase reporter activity which is dependent on Fz4 expression. TCF mediated firefly luciferase activity was normalized to renilla luciferase activity. The value for Norrin+Fz4+Lrp5 transfected cells is set to 100% and the values for the other groups are adjusted accordingly.

FIG. 11B is a bar chart representing the effects of Norrin cysteine mutations and hydrophobic residue mutations at the dimer interface on Norrin function in 293STF cells transfected with Fz4 or Fz4+Lrp5.

FIG. 11C is a bar chart representing the effects of Norrin cysteine mutations and hydrophobic residue mutations at the dimer interface on Norrin function in 293STF cells transfected with Fz4+Lrp6.

FIG. 12A depicts flow cytometry scans representing Fz4 surface expression for HEK293 cells transfected with Fz4 receptor with or without different tags. HEK293 cells were transfected with Fz4 receptor with or without a v5, YFP, YFP-N, YFP-C or Rlu tag at the C-terminus for 2 d.

FIG. 12B depicts a photomicrograph of a Western Blot analysis representing electrophoretic separation of cell extracts from HEK293 cells transfected with Fz4 treated with or without 5 μg/ml MBP-Norrin on day 2 and harvested on day 3. Co-expression with Norrin or treatment with MBP-Norrin protein did not change overall protein expression for Fz4 receptor. HEK293 cells were transfected with Fz4±Norrin on day 1.

FIG. 12C (1 & 2) depicts a photomicrographs of (1) HEK293 cells transfected with FzYFP; and (2) FzYFP and Norrin DNA. Cell surface staining indicates Fz4 surface expression. FIG. 12C (3) depicts a bar chart representing the surface fluorescence quantified as shown in FIGS. 12 C (1 & 2).

FIG. 12 D depicts photomicrographs of cell surface Fz4-YFP fluorescence of various COS-1 cell constructs transfected with Fz4-YFP, Fz4-YFP-N, Fz4-YFP-C or Fz4-YFP-N+Fz4-YFP-C. COS-1 cells were transfected with either intact YFP construct or the complementary YFP-N and YFP-C constructs.

FIG. 13A depicts a bar chart depicting the interaction between biotinylated DKK1 peptide and His8-tagged Lrp6 BP1-2 with increasing concentrations of competitor untagged MBP-Norrin protein.

FIG. 13B is a representation of a curve measuring binding kinetics between Lrp6 BP2-Fc protein and MBP-Norrin determined by biolayer interferometry.

FIG. 13C depicts a bar chart depicting the binding of MBP-Norrin to Lrp6 BP2-Fc measured by AlphaScreen assay.

FIG. 14A depicts a line graphs depicting ternary complex formation between MBP-Norrin and Fz4-CRD and different Lrp6 ECD proteins (40 μg/ml Lrp6BP1-2, 40 μg/ml Lrp6BP3-4 and 80 μg/ml Lrp6BP1-4) as measured using biolayer interferometry.

FIG. 14B depicts a line graphs depicting ternary complex formation between MBP-Norrin and Fz4-FcH6 protein and different Lrp6 ECD proteins. Fz4-FcH6 protein does not directly bind Lrp6 β-propeller domains.

FIG. 15A is a sequence alignment between different Norrin proteins from different animal species. The darker shaded areas indicate sequence identity and the lighter shaded areas indicate sequence homology. The stars denote the cysteines forming intramolecular or intermolecular disulfide bonds. The filled triangles denote the residues for Fz4 binding whereas the filled circles denote residues for Lrp5/6 binding.

FIG. 15B is a sequence alignment between human Norrin protein and various growth factors and extracellular matrix proteins.

These figures are provided by way of example and are not intended to limit the scope of the invention.

### DETAILED DESCRIPTION

### Definitions

For purposes of this disclosure, unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N. Y. 1994); Sambrook et al., Molecular Cloning, A Laboratory Manual, Cold Spring Harbor Press (Cold Spring Harbor, N. Y. 1989). These references are hereby incorporated into this disclosure by reference in their entireties.

Before the present compositions and methods are described, it is to be understood that any invention is not limited to the particular processes, compositions, or methodologies described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. It is also to be understood that the terminology used in the description is for the purpose of describing the particular versions or embodiments only, and is not intended to limit the scope of the present invention, which will be limited only by the appended claims. Unless clearly defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

It must be noted that, as used herein, and in the appended claims, the singular forms “a”, “an”, and “the” include plural reference unless the context clearly dictates otherwise.

Embodiments including the transition phrase “consisting of” or “consisting essentially of” include only the recited components and inactive ingredients. For example, a composition “consisting essentially of” a Norrin mutant polypeptide can include a Norrin mutant polypeptide and inactive excipients, which may or may not be recited, but may not contain any additional active agents or angiogenesis suppressing agents. A composition “consisting of” a Norrin mutant polypeptide may include only the components specifically recited.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the description includes both instances where the event occurs and instances where it does not.

“Administering”, when used in conjunction with a therapeutic, means to administer a therapeutic directly into or onto a target tissue or to administer a therapeutic to a subject whereby the therapeutic positively impacts the tissue to which it is targeted. “Administering” a composition may be accomplished by oral administration, injection, infusion, absorption or by any method in combination with other known techniques. “Administering” may include the act of self-administration or administration by another person such as a healthcare provider or a device.

The term “amino acid” not only encompasses the 20 common amino acids in naturally synthesized proteins, but also includes any modified, unusual, or synthetic amino acid. One of ordinary skill in the art would be familiar with modified, unusual, or synthetic amino acids.

The term “improves” is used to convey that the present invention refers to the overall physical state of an individual to whom an active agent has been administered. For example, the overall physical state of an individual may “improve” if one or more symptoms of a neurodegenerative disorder are alleviated by administration of an active agent. “Improves may also refer to changes in the appearance, form, characteristics, and/or physical attributes of tissue, or any combination thereof, to which it is being provided, applied, or administered.

As used herein, the term “therapeutic” means an agent utilized to treat, combat, ameliorate, or prevent, or any combination thereof, an unwanted condition or disease of a subject.

As used herein, the term “effective amount” refers to the amount of a composition (e.g., a Norrin mutant polypeptide) sufficient to effect beneficial or desired results. An effective amount can be administered in one or more administrations, applications or dosages and is not intended to be limited to a particular formulation or administration route. An effective amount may include a therapeutically effective amount, or a non-therapeutically effective amount.

The terms “therapeutically effective amount” or “therapeutic dose” as used herein are interchangeable and may refer to the amount of an active agent or pharmaceutical compound or composition that elicits a biological and/or medicinal response in a tissue, system, animal, individual or human that is being sought by a researcher, veterinarian, medical doctor or other clinician, or any combination thereof. A biological or medicinal response may include, for example, one or more of the following: (1) preventing a disorder, disease, or condition in an individual that may be predisposed to the disorder, disease, or condition but does not yet experience or display pathology or symptoms of the disorder, disease, or condition, (2) inhibiting a disorder, disease, or condition in an individual that is experiencing or displaying the pathology or symptoms of the disorder, disease, or condition or arresting further development of the pathology and/or symptoms of the disorder, disease, or condition, and/or (3) ameliorating a disorder, disease, or condition in an individual that is experiencing or exhibiting the pathology or symptoms of the disorder, disease, or condition or reversing the pathology and/or symptoms disorder, disease, or condition experienced or exhibited by the individual.

As used herein, the term “anti-angiogenic” or “anti-angiogenic agent” refers to the capability of any agent, compound, or pharmaceutical formulation thereof, to attenuate, inhibit or otherwise reduce angiogenesis, including in some conditions, attenuation of the migration, proliferation and differentiation of endothelial cells. Anti-angiogenic agents of the present invention may reduce or terminate formation of new blood vessels in a target tissue.

The term “treating” may be taken to mean prophylaxis of a specific disorder, disease, or condition, alleviation of the symptoms associated with a specific disorder, disease, or condition and/or prevention of the symptoms associated with a specific disorder, disease or condition. In some embodiments, the term refers to slowing the progression of the disorder, disease, or condition or alleviating the symptoms associated with the specific disorder, disease, or condition. In some embodiments, the term refers to slowing the progression of the disorder, disease, or condition. In some embodiments, the term refers to alleviating the symptoms associated with the specific disorder, disease, or condition. In some embodiments, the term refers to restoring function which was impaired or lost due to a specific disorder, disease, or condition.

The term “subject” generally refers to any living organism to which compounds described herein are administered and may include, but is not limited to, any human, primate, or non-human mammal, for example, an experimental animal or model, such as a mouse, rat, rabbit, guinea pig, hamster, ferret, dog, cat, and the like. In some embodiments, a subject may also include non-mammalian animals, or non-vertebrate animals. A “subject” may or may not be exhibiting the signs, symptoms, or pathology of aberrant angiogenesis at any stage of any embodiment.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild-type” or “native” (used interchangeably) refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may form by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “so” and a double stranded nucleic acid by the prefix “ds”.

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

The term “isolated” or “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated.

In the context of this invention the term “aberrant angiogenesis” refers to unwanted or uncontrolled angiogenesis. For example, inhibiting or reducing aberrant angiogenesis refers to a physiological response associated with the decrease or inhibition of pro-angiogenic stimuli associated with Norrin-Fz4-Lrp5/6 signaling, most commonly through the β-catenin/TCF transcriptional pathway. The term an “anti-angiogenic amount” of the compositions and compounds of the present invention are defined as the decrease or inhibition of pro-angiogenic stimuli in a tissue associated with Norrin-Fz4-Lrp5/6 signaling, most commonly through the β-catenin/TCF transcriptional pathway in the tissue by at least 10%, or at least 20%, or at least 30%, or at least 40%, or at least 50%, or at least 60%, or at least 70% or at least 80% relative to the amount of Norrin-Fz4-Lrp5/6 signaling in the same tissue in the absence of the active agent.

As used herein, the term “hypervascularization disease” refers to a disease, disorder, condition or symptom that relates to uncontrolled, disorganized blood vessel formation in tissue that results in some pathological disease or disorder.

As used herein, the term “subject diagnosed with a cancer” refers to a subject who has been tested and found to have cancerous cells, and possibly requiring oncologic intervention. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

As used herein, the term “administration” refers to the act of giving a Norrin mutant polypeptide, or a pharmaceutically acceptable salt, prodrug, or solvate thereof, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intramuscularly, intratumorally, intraperitoneally, etc.) and the like.

As used herein, the term “co-administration” refers to the administration of at least two agent(s) (e.g., a Norrin mutant polypeptide and one or more other agents such as an anti-angiogenic agent, or an anti-cancer agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., a Norrin mutant polypeptide) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as whole blood, plasma, serum and the like, and other fluids typically found within or produced by an organism, such as cerebrospinal fluid, ascites fluid, vitreous fluid and the like.

“Wnt protein signaling” or “Wnt signaling” is used herein to refer to the mechanism by which Wnt proteins modulate cell activity. Wnt proteins modulate cell activity by binding to Wnt receptor complexes that include a polypeptide from the Frizzled (Fzd or Fz) family of proteins and a polypeptide of the low-density lipoprotein receptor (LDLR)-related protein (LRP) family of proteins e.g. Lrp5 and/or Lrp6. Fzd proteins are seven-pass transmembrane proteins (Ingham, P. W. (1996) Trends Genet. 12: 382-384; YangSnyder, J. et al. (1996) Curr. Biol. 6: 1302-1306; Bhanot, P. et al. (1996) Nature 382: 225-230). There are ten known members of the Fzd family (Fzd1 through Fzd10), any of which may be used in the Wnt receptor complex. LRP proteins are single-pass transmembrane proteins that bind and internalize ligands in the process of receptor-mediated endocytosis; LRP family members Lrp5 (NCBI RefSeq.: NM_{—}002335.2) or Lrp6 (NCBI RefSeq.: NM_{—}002336.2) are included in the Wnt receptor complex.

Once activated by Wnt binding, the Wnt receptor complex will activate one or more intracellular signaling cascades. These include the canonical Wnt signaling pathway; the Wnt/planar cell polarity (Wnt/PCP) pathway; and the Wnt-calcium (Wnt/Ca²⁺) pathway (Giles, R H et al. (2003) Biochim Biophys Acta 1653, 1-24; Peifer, M. et al. (1994) Development 120: 369-380; Papkoff, J. et al (1996) Mol. Cell Biol. 16: 2128-2134; Veeman, M. T. et al. (2003) Dev. Cell 5: 367-377). For example, activation of the canonical Wnt signaling pathway results in the inhibition of phosphorylation of the intracellular protein β-catenin, leading to an accumulation of β-catenin in the cytosol and its subsequent translocation to the nucleus where it interacts with transcription factors, e.g. TCF/LEF, to activate target genes.

The phrases “Wnt-mediated condition” and “Wnt-mediated disorder” are used interchangeably herein to describe a condition, disorder, or disease state characterized by aberrant Wnt signaling. In a specific aspect, the aberrant Wnt signaling is a level of Wnt signaling in a cell or tissue suspected of being diseased that exceeds the level of Wnt signaling in a similar non-diseased cell or tissue. Examples of Wnt-mediated disorders include those associated with aberrant angiogenesis, e.g. retinopathies, and those associated with aberrant proliferation, e.g. cancer.

### Abbreviations:

AMD: Age-related Macular Degeneration; BMP: bone morphogenetic protein; BRET: Bioluminescence Resonance Energy Transfer; CG: Chorionic Gonadotropin; CRD: Cysteine Rich Domain (of Frizzled receptors); ECD: Extra-Cellular Domains; EM: Electron Microscopy; FEVR: Familial Exudative Vitreo-Retinopathy; Fz4: Frizzled protein 4; hCG: Lrp: Low density lipoprotein receptor-Related Protein; luc: luciferase; MBP: Maltose-Binding Protein; ND: Norrie disease; NGF: Nerve Growth Factor, PDGF: Platelet-Derived Growth Factor Rlu: Renilla luciferase; ROP: Retinopathy Of Prematurity; TCF: T Cell Factor; TGF-β: Transforming Growth Factor-β; YFP: Yellow Fluorescent Protein.

### A. Norrin Mutant Polypeptides

In some embodiments of the present invention, the inventors have discovered a method to express, purify, crystallize, and determine the structure of the polypeptide or protein Norrin. Key mutational studies provided herein have revealed specific amino acid sequence mutations in Norrin that disrupt Norrin signaling pathways that can be exploited to treat specific diseases that result in aberrant angiogenic signaling as a result of Wnt and Norrin mediated angiogenesis.

A “native amino acid sequence or wild-type amino acid sequence of Norrin” comprises a polypeptide having the same amino acid sequence as the corresponding Norrin polypeptide derived from nature. In one embodiment, a native or wild-type Norrin polypeptide comprises the amino acid sequence of SEQ ID NO:1 (see Table 1).

A “Norrin mutant polypeptide”, means a native Norrin polypeptide or fragment thereof, having at least about 80%, 85%, 90%, 91%, 92%, 93% 94%, 95%, 96%, 97%, 98% or 99% amino acid sequence identity with any of the native sequence Norrin polypeptide sequences as disclosed herein. In some embodiments, the Norrin mutant polypeptide having one or more amino acid residues that are mutated relative to a wild-type or native Norrin amino acid sequence as provided in Table 1. Ordinarily, a Norrin mutant polypeptide will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native or wild-type sequence Norrin polypeptide sequence as disclosed herein. In some embodiments, Norrin mutant polypeptides are at least about 10 amino acids in length, alternatively at least about 20, 30, 40, 50, 60, 70, 80, 90, 100, 110, 120, 130, 140 amino acids or 150 amino acids in length, or more. All Norrin mutant polypeptides including functional fragments thereof, bind to at least one of Fz4, or Lrp5, and Lrp6, but fails to form an angiogenic functional or activated Fz4-Lrp5/6 ternary complex in the presence of Fz4 and Lrp5/6. In some embodiments, the Norrin mutant polypeptides of the present invention has at least one amino acid mutation in the amino acid sequence of human Norrin of SEQ ID NO:1 as provided in Table 1.

In some embodiments, isolated Norrin mutant polypeptides of the present invention have an amino acid sequence that contains one or more amino acid substitutions at the substituted positions indicated by an X, J, or Z shown in Table 1. As used herein, Norrin mutant polypeptides having one or more mutations refers to the polypeptides that have the primary amino acid sequence of SEQ ID NO:1, in which one or more amino acids in the amino acid sequence of SEQ ID NO:1 are substituted with a different amino acid. In some embodiments illustrative Norrin mutant polypeptides are set forth in Table 1. In some embodiments, Norrin mutant polypeptides can have one to seven amino acid substitutions, or one to five amino acid substitutions or one to three amino acid substitutions at the positions at X, J, or Z as shown in Table 1 relative to the amino acid sequence of SEQ ID NO:1. For example, in one embodiment, the Norrin mutant polypeptide has three amino acid substitutions, the polypeptide having a substitution as provided in the amino acid sequence of SEQ ID NO:2 wherein X is glycine, and further contains the substitutions of SEQ ID NO:9, wherein X is alanine, and a substitution of SEQ ID NO:21, wherein X is glutamic acid. In another example, an illustrative Norrin mutant polypeptide has 6 amino acid substitutions relative to the amino acid sequence of SEQ ID NO:1, comprising the substitutions of amino acid sequence of SEQ ID NO: 14, wherein X is alanine, Z is tryptophan, and J is glycine, the substitutions of amino acid sequence of SEQ ID NO:24, wherein Z is methionine and X is glutamic acid, and the substitutions of amino acid sequence of SEQ ID NO:28, wherein X is lysine.

In some embodiments, an isolated Norrin mutant polypeptide comprises the amino acid sequence of SEQ ID NO: 1, wherein the sequence has one or more amino acid substitutions at positions: 93, 95, 131, 89, 123, 41, 43, 44, 45, 59, 60, 61, 120, 121, 122, 52, 53, 54, 107, 109, 115, 55, and 110 relative to SEQ ID NO: 1.

In accordance with the present invention, illustrative isolated Norrin mutant polypeptides include a Norrin mutant polypeptide comprising the amino acid sequence of SEQ ID NO: 1, wherein the polypeptide has two to seven, two to five, two to four, one to three, three, two, or one amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1. In some embodiments, the Norrin mutant polypeptide has an amino acid sequence having one or more amino acid substitutions at positions: 131, 59, 122, 52, 53, 93, 107, and 109 of SEQ ID NO:1. In some embodiments, the isolated Norrin mutant polypeptide has one or more of the following amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another embodiment, the isolated Norrin mutant polypeptide can have two or more amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, for example, a substitution: C93A, C131A, M59A, Y122A, L52A, Y53A, and R107E, or for example, M59A, Y122A, L52A, Y53A, and R107E. In one example, the Norrin mutant polypeptide an amino acid sequence having one to seven of the following amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In one embodiment, any of the foregoing Norrin mutant polypeptides has one or more amino acid substitutions wherein the replacement amino acid is a conservative amino acid, and thus, the substitution is a conservative amino acid substitution.

In one embodiment, the Norrin mutant polypeptides described herein has one to seven amino acid substitutions at amino acid positions as described in Table 1, wherein the amino acid replacement is a conservative amino acid, and thus, the substitution is a conservative amino acid substitution. For example, an isolated Norrin mutant polypeptide includes a polypeptide having an amino acid sequence in which the amino acid substitution occurs at positions: 93, 131, 59, 122, 52, 53, 107 in SEQ ID NO: 1. In some embodiments, these substitutions at the noted positions are conservative amino acid substitutions.

In another aspect, the present invention provides an isolated Norrin mutant polypeptide wherein the polypeptide comprises an amino acid sequence of SEQ ID NOs: 2-38 and 60-62, for example, an amino acid sequence of SEQ ID NO:2, 4, 15, 23, 26, 27 or 33. In accordance with the present invention, in another aspect, the invention provides an isolated Norrin mutant polypeptide, wherein the polypeptide has an amino acid sequence having one or more amino acid substitutions as shown in Table 1. In one such example, the polypeptide comprising the amino acid sequence of SEQ ID NO: 1, and having one or more amino acid substitutions at positions 93, 131, 59, 122, 52, 53 107, and 109. In one example, an isolated Norrin mutant polypeptide has an amino acid sequence having one or more amino acid substitutions relative to SEQ ID NO:1 selected from the group consisting of: C93A, C95A, C131A, F89R, R41E, H43A, Y44A, V45A, M59A, L61A, Y120A, R121A, Y122A, L52A, Y53A, K54A, R107E, R109E, and R115E. For example, in one embodiment, the isolated Norrin mutant polypeptide has an amino acid sequence having one, or two or more amino acid substitutions: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another example, the isolated Norrin mutant polypeptide has an amino acid sequence having two to seven amino acid substitutions selected from: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or two to five amino acid substitutions relative to SEQ ID NO:1, selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or one, two or three amino acid substitutions relative to SEQ ID NO:1 selected from C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E.

In accordance with the present invention, in one aspect, the invention provides an isolated Norrin mutant polypeptide, wherein the polypeptide has an amino acid sequence having one to seven amino acid substitutions relative to SEQ ID NO:1 comprising: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In various aspects, an exemplary isolated Norrin mutant polypeptide has an amino acid substitution as defined above, wherein the amino acid substitution or substitutions is or are, conservative amino acid substitutions.

Conservative amino acid replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, WH Freeman and Co., 1981). Whether a change in the amino acid sequence of a peptide results in a functional Norrin mutant polypeptide (e.g. functional in the sense that the resulting polypeptide mimics or antagonizes the wild-type form) can be readily determined by assessing the ability of the Norrin mutant polypeptide to produce a response in cells in a fashion similar to the wild-type protein, or competitively inhibit such a response. Polypeptides in which more than one amino acid replacement has taken place can readily be tested in the same manner.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, as described in U.S. Pat. No. 6,828,146.

In some embodiments, exemplary Norrin mutant polypeptides of SEQ ID NOs: 2-38 and 60-62 are provided in Table 1 below.

TABLE 1
Norrin polypeptides and nucleic acids.

| SEQ | | |
|---|---|---|
| ID NO | Amino Acid or Nucleotide Sequence | Comment/Mutation |
| 1 | MRKHVLAASF SMLSLLVIMG DTDSKTDSSF IMDSDPRRCM | Wild-type Human pre- |
| | RHHYVDSISH PLYKCSSKMV LLARCEGHCS QASRSEPLVS | cursor Norrin (133 a.a.) |
| | FSTVLKQPFR SSCHCCRPQT SKLKALRLRC SGGMRLTATY | |
| | RYILSCHCEE CNS | |
| 2 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than C and A |
| | FSTVLKQPFR SSXHCCRPQT SKLKALRLRC SGGMRLTATY | |
| | RYILSCHCEE CNS | |
| 3 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than C and A |
| | FSTVLKQPFR SSCHXCRPQT SKLKALRLRC SGGMRLTATY | |
| | RYILSCHCEE CNS | |
| 4 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than C and A |
| | FSTVLKQPFR SSCHCCRPQT SKLKALRLRC SGGMRLTATY | |
| | RYILSCHCEE XNS | |
| 5 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than F and L |
| | FSTVLKQPXR SSCHCCRPQT SKLKALRLRC SGGMRLTATY | |
| | RYILSCHCEE CNS | |
| 6 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than I |
| | FSTVLKQPFR SSCHCCRPQT SKLKALRLRC SGGMRLTATY | |
| | RYXLSCHCEE CNS | |
| 7 | MRKHVLAASF SMLSLLVIMG DTDSKTDSSF IMDSDPRRCM | X is any amino acid other |
| | XHHYVDSISH PLYKCSSKMV LLARCEGHCS QASRSEPLVS | than R, K, T, and S |
| | FSTVLKQPFR SSCHCCRPQT SKLKALRLRC SGGMRLTATY | |
| | RYILSCHCEE CNS | |
| 8 | MRKHVLAASF SMLSLLVIMG DTDSKTDSSF IMDSDPRRCM | X is any amino acid other |
| | RHXYVDSISH PLYKCSSKMV LLARCEGHCS QASRSEPLVS | than H, R and Q |
| | FSTVLKQPFR SSCHCCRPQT SKLKALRLRC SGGMRLTATY | |
| | RYILSCHCEE CNS | |
| 9 | MRKHVLAASF SMLSLLVIMG DTDSKTDSSF IMDSDPRRCM | X is any amino acid other |
| | RHHXVDSISH PLYKCSSKMV LLARCEGHCS QASRSEPLVS | than Y and C |
| | FSTVLKQPFR SSCHCCRPQT SKLKALRLRC SGGMRLTATY | |
| | RYILSCHCEE CNS | |
| 10 | MRKHVLAASF SMLSLLVIMG DTDSKTDSSF IMDSDPRRCM | X is any amino acid other |
| | RHHYXDSISH PLYKCSSKMV LLARCEGHCS QASRSEPLVS | than V, M and E |
| | FSTVLKQPFR SSCHCCRPQT SKLKALRLRC SGGMRLTATY | |
| | RYILSCHCEE CNS | |
| 11 | MRKHVLAASF SMLSLLVIMG DTDSKTDSSF IMDSDPRRCM | X is any amino acid other |
| | RHXZVDSISH PLYKCSSKMV LLARCEGHCS QASRSEPLVS | than H, R and Q; and Z is |
| | FSTVLKQPFR SSCHCCRPQT SKLKALRLRC SGGMRLTATY | any amino acid other than |
| | RYILSCHCEE CNS | Y and C |
| 12 | MRKHVLAASF SMLSLLVIMG DTDSKTDSSF IMDSDPRRCM | X is any amino acid other |
| | RHXYZDSISH PLYKCSSKMV LLARCEGHCS QASRSEPLVS | than H, R and Q; Z is any |
| | FSTVLKQPFR SSCHCCRPQT SKLKALRLRC SGGMRLTATY | amino acid other than V, |
| | RYILSCHCEE CNS | M and E |
| 13 | MRKHVLAASF SMLSLLVIMG DTDSKTDSSF IMDSDPRRCM | Z is any amino acid other |
| | RHHZXDSISH PLYKCSSKMV LLARCEGHCS QASRSEPLVS | than Y and C; |
| | FSTVLKQPFR SSCHCCRPQT SKLKALRLRC SGGMRLTATY | X is any amino acid other |
| | RYILSCHCEE CNS | than V, M and E |
| 14 | MRKHVLAASF SMLSLLVIMG DTDSKTDSSF IMDSDPRRCM | X is any amino acid other |
| | RHXZJDSISH PLYKCSSKMV LLARCEGHCS QASRSEPLVS | than H, R and Q; Z is any |
| | FSTVLKQPFR SSCHCCRPQT SKLKALRLRC SGGMRLTATY | amino acid other than Y |
| | RYILSCHCEE CNS | and C; J is any amino |
| | | acid other than V, M and |
| | | E |
| 15 | MRKHVLAASF SMLSLLVIMG DTDSKTDSSF IMDSDPRRCM | X is any amino acid other |
| | RHHYVDSISH PLYKCSSKXV LLARCEGHCS QASRSEPLVS | than M |
| | FSTVLKQPFR SSCHCCRPQT SKLKALRLRC SGGMRLTATY | |
| | RYILSCHCEE CNS | |
| 16 | MRKHVLAASF SMLSLLVIMG DTDSKTDSSF IMDSDPRRCM | X is any amino acid other |
| | RHHYVDSISH PLYKCSSKMX LLARCEGHCS QASRSEPLVS | than V and E |
| | FSTVLKQPFR SSCHCCRPQT SKLKALRLRC SGGMRLTATY | |
| | RYILSCHCEE CNS | |
| 17 | MRKHVLAASF SMLSLLVIMG DTDSKTDSSF IMDSDPRRCM | X is any amino acid other |
| | RHHYVDSISH PLYKCSSKMV XLARCEGHCS QASRSEPLVS | than L, F, I and P |
| | FSTVLKQPFR SSCHCCRPQT SKLKALRLRC SGGMRLTATY | |
| | RYILSCHCEE CNS | |
| 18 | MRKHVLAASF SMLSLLVIMG DTDSKTDSSF IMDSDPRRCM | X is any amino acid other |
| | RHHYVDSISH PLYKCSSKXJ LLARCEGHCS QASRSEPLVS | than M; |
| | FSTVLKQPFR SSCHCCRPQT SKLKALRLRC SGGMRLTATY | J is any amino acid other |
| | RYILSCHCEE CNS | than V and E |
| 19 | MRKHVLAASF SMLSLLVIMG DTDSKTDSSF IMDSDPRRCM | X is any amino acid other |
| | RHHYVDSISH PLYKCSSKXV JLARCEGHCS QASRSEPLVS | than M; |
| | FSTVLKQPFR SSCHCCRPQT SKLKALRLRC SGGMRLTATY | J is any amino acid other |
| | RYILSCHCEE CNS | than L, F, I and P |
| 20 | MRKHVLAASF SMLSLLVIMG DTDSKTDSSF IMDSDPRRCM | X is any amino acid other |
| | RHHYVDSISH PLYKCSSKMX JLARCEGHCS QASRSEPLVS | than V and E; J is any |
| | FSTVLKQPFR SSCHCCRPQT SKLKALRLRC SGGMRLTATY | amino acid other than L, |
| | RYILSCHCEE CNS | F, I and P |
| 21 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than Y and C |
| | FSTVLKQPFR SSCHCCRPQT SKLKALRLRC SGGMRLTATX | |
| | RYILSCHCEE CNS | |
| 22 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R, G, W, Q and L |
| | FSTVLKQPFR SSCHCCRPQT SKLKALRLRC SGGMRLTATY | |
| | XYILSCHCEE CNS | |
| 23 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than Y |
| | FSTVLKQPFR SSCHCCRPQT SKLKALRLRC SGGMRLTATY | |
| | RXILSCHCEE CNS | |
| 24 | MRKHVLAASF SMLSLLVIMG DTDSKTDSSF IMDSDPRRCM | Z is any amino acid other |
| | RHHYVDSISH PLYKCSSKMV LLARCEGHCS QASRSEPLVS | than Y and C; |
| | FSTVLKQPFR SSCHCCRPQT SKLKALRLRC SGGMRLTATZ | X is any amino acid other |
| | XYILSCHCEE CNS | than R, G, W, Q and L |
| 25 | MRKHVLAASF SMLSLLVIMG DTDSKTDSSF IMDSDPRRCM | Z is any amino acid other |
| | RHHYVDSISH PLYKCSSKMV LLARCEGHCS QASRSEPLVS | than Y and C; X is any |
| | FSTVLKQPFR SSCHCCRPQT SKLKALRLRC SGGMRLTATZ | amino acid other than Y |
| | RXILSCHCEE CNS | |
| 26 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R, G, W, Q and L; J |
| | FSTVLKQPFR SSCHCCRPQT SKLKALRLRC SGGMRLTATY | is any amino acid other |
| | XJILSCHCEE CNS | than Y |
| 27 | MRKHVLAASF SMLSLLVIMG DTDSKTDSSF IMDSDPRRCM | X is any amino acid other |
| | RHHYVDSISH PXYKCSSKMV LLARCEGHCS QASRSEPLVS | than L |
| | FSTVLKQPFR SSCHCCRPQT SKLKALRLRC SGGMRLTATY | |
| | RYILSCHCEE CNS | |
| 28 | MRKHVLAASF SMLSLLVIMG DTDSKTDSSF IMDSDPRRCM | X is any amino acid other |
| | RHHYVDSISH PLXKCSSKMV LLARCEGHCS QASRSEPLVS | than Y |
| | FSTVLKQPFR SSCHCCRPQT SKLKALRLRC SGGMRLTATY | |
| | RYILSCHCEE CNS | |
| 29 | MRKHVLAASF SMLSLLVIMG DTDSKTDSSF IMDSDPRRCM | X is any amino acid other |
| | RHHYVDSISH PLYXCSSKMV LLARCEGHCS QASRSEPLVS | than K and N |
| | FSTVLKQPFR SSCHCCRPQT SKLKALRLRC SGGMRLTATY | |
| | RYILSCHCEE CNS | |
| 30 | MRKHVLAASF SMLSLLVIMG DTDSKTDSSF IMDSDPRRCM | X is any amino acid other |
| | RHHYVDSISH PXJKCSSKMV LLARCEGHCS QASRSEPLVS | than L; |
| | FSTVLKQPFR SSCHCCRPQT SKLKALRLRC SGGMRLTATY | J is any amino acid other |
| | RYILSCHCEE CNS | than Y |
| 31 | MRKHVLAASF SMLSLLVIMG DTDSKTDSSF IMDSDPRRCM | X is any amino acid other |
| | RHHYVDSISH PXYJCSSKMV LLARCEGHCS QASRSEPLVS | than L; |
| | FSTVLKQPFR SSCHCCRPQT SKLKALRLRC SGGMRLTATY | J is any amino acid other |
| | RYILSCHCEE CNS | than K and N |
| 32 | MRKHVLAASF SMLSLLVIMG DTDSKTDSSF IMDSDPRRCM | X is any amino acid other |
| | RHHYVDSISH PLXJCSSKMV LLARCEGHCS QASRSEPLVS | than Y; |
| | FSTVLKQPFR SSCHCCRPQT SKLKALRLRC SGGMRLTATY | J is any amino acid other |
| | RYILSCHCEE CNS | than K and N |
| 33 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R |
| | FSTVLKQPFR SSCHCCRPQT SKLKALXLRC SGGMRLTATY | |
| | RYILSCHCEE CNS | |
| 34 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R |
| | FSTVLKQPFR SSCHCCRPQT SKLKALRLXC SGGMRLTATY | |
| | RYILSCHCEE CNS | |
| 35 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R and L |
| | FSTVLKQPFR SSCHCCRPQT SKLKALRLRC SGGMXLTATY | |
| | RYILSCHCEE CNS | |
| 36 | MRKHVLAASF SMLSLLVIMG DTDSKTDSSF IMDSDPRRCM | X is any amino acid other |
| | RHHYVDSISH PLYKCSSKMV LLARCEGHCS QASRSEPLVS | than R; Z is any amino |
| | FSTVLKQPFR SSCHCCRPQT SKLKALRLXC SGGMZLTATY | acid other than R and L |
| | RYILSCHCEE CNS | |
| 37 | MRKHVLAASF SMLSLLVIMG DTDSKTDSSF IMDSDPRRCM | X is any amino acid other |
| | RHHYVDSISH PLYXCSSKMV LLARCEGHCS QASRSEPLVS | than K and N; Z is any |
| | FSTVLKQPFR SSCHCCRPQT SKLKALRLZC SGGMRLTATY | amino acid other than R |
| | RYILSCHCEE CNS | |
| 38 | MRKHVLAASF SMLSLLVIMG DTDSKTDSSF IMDSDPRRCM | X is any amino acid other |
| | RHHYVDSISH PLYKXSSKMV LLARCEGHCS QASRSEPLVS | than C, R, and F; Z is |
| | FSTVLKQPFR SSCHCCRPQT SKLKALRLRZ SGGMRLTATY | any amino acid other than |
| | RYILSCHCEE CNS | C, R, G, and S |
| 39 | AAAACGGACA GCTCATTCAT AATGGACTCG GACCCTCGAC | Mature Human Norrin DNA |
| | GCTGCATGAG GCACCACTAT GTGGATTCTA TCAGTCACCC | sequence of SEQ ID NO: 41 |
| | ATTGTACAAG TGTAGCTCAA AGATGGTGCT CCTGGCCAGG | |
| | TGCGAGGGGC ACTGCAGCCA GGCGTCACGC TCCGAGCCTT | |
| | TGGTGTCGTT CAGCACTGTC CTCAAGCAAC CCTTCCGTTC | |
| | CTCCTGTCAC TGCTGCCGGC CCCAGACTTC CAAGCTGAAG | |
| | GCACTGCGGC TGCGATGCTC AGGGGGCATG CGACTCACTG | |
| | CCACCTACCG GTACATCCTC TCCTGTCACT GCGAGGAATG | |
| | CAATTCC | |
| 40 | MAKIEEGKLV IWINGDKGYN GLAEVGKKFE KDTGIKVTVE | Recombinant MBP-Norrin |
| | HPDKLEEKFP QVAATGDGPD IIFWAHDRFG GYAQSGLLAE | protein sequence |
| | ITPDKAFQDK LYPFTWDAVR YNGKLIAYPI AVEALSLIYN | |
| | KDLLPNPPKT WEEIPALDKE LKAKGKSALM FNLQEPYFTW | |
| | PLIAADGGYA FKYENGKYDI KDVGVDNAGA KAGLTFLVDL | |
| | IKNKHMNADT DYSIAEAAFN KGETAMTING PWAWSNIDTS | |
| | KVNYGVTVLP TFKGQPSKPF VGVLSAGINA ASPNKELAKE | |
| | FLENYLLTDE GLEAVNKDKP LGAVALKSYE EELAKDPRIA | |
| | ATMENAQKGE IMPNIPQMSA FWYAVRTAVI NAASGRQTVD | |
| | EALKDAQTNA AAEFKTDSSF IMDSDPRRCM RHHYVDSISH | |
| | PLYKCSSKMV LLARCEGHCS QASRSEPLVS FSTVLKQPFR | |
| | SSCHCCRPQT SKLKALRLRC SGGMRLTATY RYILSCHCEE | |
| | CNS | |
| 41 | KTDSSFIMDS DPRRCMRHHY VDSISHPLYK CSSKMVLLAR | Mature Human Norrin |
| | CEGHCSQASR SEPLVSFSTV LKQPFRSSCH CCRPQTSKLK | protein sequence |
| | ALRLRCSGGM RLTATYRYIL SCHCEECNS | |
| 42 | MAKIEEGKLV IWINGDKGYN GLAEVGKKFE KDTGIKVTVE | MBP protein sequence |
| | HPDKLEEKFP QVAATGDGPD IIFWAHDRFG GYAQSGLLAE | |
| | ITPDKAFQDK LYPFTWDAVR YNGKLIAYPI AVEALSLIYN | |
| | KDLLPNPPKT WEEIPALDKE LKAKGKSALM FNLQEPYFTW | |
| | PLIAADGGYA FKYENGKYDI KDVGVDNAGA KAGLTFLVDL | |
| | IKNKHMNADT DYSIAEAAFN KGETAMTING PWAWSNIDTS | |
| | KVNYGVTVLP TFKGQPSKPF VGVLSAGINA ASPNKELAKE | |
| | FLENYLLTDE GLEAVNKDKP LGAVALKSYE EELAKDPRIA | |
| | ATMENAQKGE IMPNIPQMSA FWYAVRTAVI NAASGRQTVD | |
| | EALKDAQTNA AAEF | |
| 43 | atggcaaaaa tcgaagaagg taaactggta atctggatta | MBP-Human Norrin fusion |
| | acggcgataa aggctataac ggtctcgctg aagtcggtaa | DNA sequence |
| | gaaattcgag aaagataccg gaattaaagt caccgttgag | |
| | catccggata aactggaaga gaaattccca caggttgcgg | |
| | caactggcga tggccctgac attatcttct gggcacacga | |
| | ccgctttggt ggctacgctc aatctggcct gttggctgaa | |
| | atcaccccgg acaaagcgtt ccaggacaag ctgtatccgt | |
| | ttacctggga tgccgtacgt tacaacggca agctgattgc | |
| | ttacccgatc gctgttgaag cgttatcgct gatttataac | |
| | aaagatctgc tgccgaaccc gccaaaaacc tgggaagaga | |
| | tcccggcgct ggataaagaa ctgaaagcga aaggtaagag | |
| | cgcgctgatg ttcaacctgc aagaaccgta cttcacctgg | |
| | ccgctgattg ctgctgacgg gggttatgcg ttcaagtatg | |
| | aaaacggcaa gtacgacatt aaagacgtgg gcgtggataa | |
| | cgctggcgcg aaagcgggtc tgaccttcct ggttgacctg | |
| | attaaaaaca aacacatgaa tgcagacacc gattactcca | |
| | tcgcagaagc tgcctttaat aaaggcgaaa cagcgatgac | |
| | catcaacggc ccgtgggcat ggtccaacat cgacaccagc | |
| | aaagtgaatt atggtgtaac ggtactgccg accttcaagg | |
| | gtcaaccatc caaaccgttc gttggcgtgc tgagcgcagg | |
| | tattaacgcc gccagtccga acaaagagct ggcgaaagag | |
| | ttcctcgaaa actatctgct gactgatgaa ggtctggaag | |
| | cggttaataa agacaaaccg ctgggtgccg tagcgctgaa | |
| | gtcttacgag gaagagttgg cgaaagatcc acgtattgcc | |
| | gccactatgg aaaacgccca gaaaggtgaa atcatgccga | |
| | acatcccgca gatgtccgct ttctggtatg ccgtgcgtac | |
| | tgcggtgatc aacgccgcca gcggtcgtca gactgtcgat | |
| | gaagccctga aagacgcgca gactaacgcg gcggccgaat | |
| | tcaaaacgga cagctcattc ataatggact cggaccctcg | |
| | acgctgcatg aggcaccact atgtggattc tatcagtcac | |
| | ccattgtaca agtgtagctc aaagatggtg ctcctggcca | |
| | ggtgcgaggg gcactgcagc caggcgtcac gctccgagcc | |
| | tttggtgtcg ttcagcactg tcctcaagca acccttccgt | |
| | tcctcctgtc actgctgccg gccccagact tccaagctga | |
| | aggcactgcg gctgcgatgc tcagggggca tgcgactcac | |
| | tgccacctac cggtacatcc tctcctgtca ctgcgaggaa | |
| | tgcaattcct ga | |
| 44 | atggcaaaaa tcgaagaagg taaactggta atctggatta | MBP-DNA sequence |
| | acggcgataa aggctataac ggtctcgctg aagtcggtaa | |
| | gaaattcgag aaagataccg gaattaaagt caccgttgag | |
| | catccggata aactggaaga gaaattccca caggttgcgg | |
| | caactggcga tggccctgac attatcttct gggcacacga | |
| | ccgctttggt ggctacgctc aatctggcct gttggctgaa | |
| | atcaccccgg acaaagcgtt ccaggacaag ctgtatccgt | |
| | ttacctggga tgccgtacgt tacaacggca agctgattgc | |
| | ttacccgatc gctgttgaag cgttatcgct gatttataac | |
| | aaagatctgc tgccgaaccc gccaaaaacc tgggaagaga | |
| | tcccggcgct ggataaagaa ctgaaagcga aaggtaagag | |
| | cgcgctgatg ttcaacctgc aagaaccgta cttcacctgg | |
| | ccgctgattg ctgctgacgg gggttatgcg ttcaagtatg | |
| | aaaacggcaa gtacgacatt aaagacgtgg gcgtggataa | |
| | cgctggcgcg aaagcgggtc tgaccttcct ggttgacctg | |
| | attaaaaaca aacacatgaa tgcagacacc gattactcca | |
| | tcgcagaagc tgcctttaat aaaggcgaaa cagcgatgac | |
| | catcaacggc ccgtgggcat ggtccaacat cgacaccagc | |
| | aaagtgaatt atggtgtaac ggtactgccg accttcaagg | |
| | gtcaaccatc caaaccgttc gttggcgtgc tgagcgcagg | |
| | tattaacgcc gccagtccga acaaagagct ggcgaaagag | |
| | ttcctcgaaa actatctgct gactgatgaa ggtctggaag | |
| | cggttaataa agacaaaccg ctgggtgccg tagcgctgaa | |
| | gtcttacgag gaagagttgg cgaaagatcc acgtattgcc | |
| | gccactatgg aaaacgccca gaaaggtgaa atcatgccga | |
| | acatcccgca gatgtccgct ttctggtatg ccgtgcgtac | |
| | tgcggtgatc aacgccgcca gcggtcgtca gactgtcgat | |
| | gaagccctga aagacgcgca gactaacgcg gcggccgaat | |
| | tc | |
| 60 | MRKHVLAASF SMLSLLVIMG DTDSKTDSSF IMDSDPRRCM | X is any other amino acid |
| | RHHYVDSISH PLYKXSSKMV LLARCEGHCS QASRSEPLVS | other than C, R, and F |
| | FSTVLKQPFR SSCHCCRPQT SKLKALRLRC SGGMRLTATY | |
| | RYILSCHCEE CNS | |
| 61 | MRKHVLAASF SMLSLLVIMG DTDSKTDSSF IMDSDPRRCM | X is any other amino acid |
| | RHHYVDSISH PLYKCSSKMV LLARXEGHCS QASRSEPLVS | other than C, W, and Y |
| | FSTVLKQPFR SSCHCCRPQT SKLKALRLRC SGGMRLTATY | |
| | RYILSCHCEE CNS | |
| 62 | MRKHVLAASF SMLSLLVIMG DTDSKTDSSF IMDSDPRRCM | X is any other amino acid |
| | RHHYVDSISH PLYKCSSKMV LLARCEGHCS QASRSEPLVS | other than C, R, G, and S |
| | FSTVLKQPFR SSCHCCRPQT SKLKALRLRX SGGMRLTATY | |
| | RYILSCHCEE CNS | |

In some embodiments, the Norrin mutant polypeptides encompass full length (133 amino acids) mutated polypeptides based on SEQ ID NO: 1, or polypeptides that are between 10 amino acids and 150 amino acids in length. Norrin mutant polypeptides include polypeptides that have an amino acid sequence comprising any portion of SEQ ID NO:1 having one or more mutations defined in Table 1. In some embodiments, the Norrin mutant polypeptides include the polypeptides of SEQ ID NOs: 2-38 and 60-62 excluding the first (or N-terminal) 24 amino acids MRKHVLAASF SMLSLLVIMG DTDS. In one embodiment, a Norrin mutant polypeptide can include a peptide or polypeptide having an amino acid length ranging from 10 to 150 amino acids, in which the amino acid sequence comprises between 10 to 133 contiguous amino acids of SEQ ID NO: 1 and comprises at least one mutation selected from Table 1. In some embodiments, a Norrin mutant polypeptide can include a peptide or polypeptide having an amino acid length ranging from 10 to 133 amino acids, in which the amino acid sequence comprises 10 to 133 contiguous amino acids of SEQ ID NO: 1 and comprises at least one mutation selected from C93A, C131A, M59A, Y122A, L52A, Y53A, and R107E relative to SEQ ID NO: 1. In some embodiments, an isolated Norrin mutant polypeptides comprises the amino acid sequence of SEQ ID NOs: 2, 4, 15, 23, 26, 27 or 33. In some embodiments, the Norrin mutant polypeptides comprising at least one mutation selected from C93A, C95A, C131A, F89R, I123N, R41E, H43A, Y44A, V45A, M59A, V60A, L61A, Y120A, R121A, Y122A, L52A, Y53A, K54A, R107E, R109E, R115E, C55A, and C110A relative to SEQ ID NO:1. In some embodiments, the Norrin mutant polypeptides comprises at least one mutation, preferably, one to seven, or one to five, or one to three, or one, or two, or three amino acid substitutions selected from C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E relative to SEQ ID NO: 1.

The isolated Norrin mutant polypeptides of the present technology can also be obtained recombinantly by expressing a nucleic acid in an expression vector, using standard and well established techniques known in the field of molecular biology. In this regard, the practice of the present technology will employ, unless otherwise indicated, conventional techniques of molecular biology, microbiology, recombinant DNA technology and immunology, which are within the skill of those working in the art. Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984): Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986) which are all incorporated by reference herein in their entireties.

In various embodiments, Norrin mutant polypeptides are recombinantly produced.

### B. Norrin Mutant Polypeptide Fusion Proteins

In some embodiments, Norrin mutant polypeptides described above further include a marker peptide or polypeptide that is fused in frame to a Norrin mutant polypeptide at either the N-terminus or the C-terminus of a Norrin mutant polypeptide described above. In some embodiments, the fusion protein comprises a Norrin mutant polypeptide fused to maltose binding protein (MBP), glutathione S-transferase (GST) tag, a 6×-His, 8×-His tags and a FLAG-tag among others. In some embodiments, the fusion protein is bacterial MBP, for example, an E. coli maltose binding protein. In some embodiments, the MBP is a protein with an amino acid sequence of SEQ ID NO:42. In some embodiments, with reference to SEQ ID NO:1, the natural 24 amino acid N-terminal Norrin signal peptide is replaced with a signal peptide from a different protein, tissue, or organism to facilitate expression and/or isolation when expressed in a non-human cell line. In one embodiment, Norrin mutant polypeptides are fused to a maltose binding protein, for example a bacterial maltose binding protein. In one embodiment, the maltose binding protein is an E. coli 392 amino acid maltose binding protein having an amino acid sequence as provided in NCBI Accession No. ABO28850, version ABO28850.1, GI:129278846. In another embodiment, the MBP-Norrin fusion protein has an amino acid sequence of SEQ ID NO:40, and a polynucleotide sequence of SEQ ID NO:43. In another embodiment, illustrative MBP-Norrin fusion proteins can include a Norrin construct fused in frame to a MBP protein, at either the N-terminus and/or the C-terminus. As used herein, a Norrin construct is a collective term to encompass both wild-type Norrin proteins and Norrin mutant polypeptides as disclosed herein. In some embodiments, an illustrative Norrin construct can include any wild-type Norrin protein or a Norrin mutant polypeptide as disclosed in Table 1. In some of these embodiments, a Norrin construct can be fused with a MBP protein from any species, for example, a bacterial MBP, which are known in the art and are readily identifiable, both at the amino acid sequence level and the nucleotide level using various molecular biology databases such as Pubmed, BLAST, Expasy and the like. In some embodiments, an illustrative bacterial MBP can include an Escherichia coli MBP. In some embodiments, a representative E. coli MBP is provided in Table 1 as SEQ ID NO: 42. Such recombinant MBP encoding nucleic acids can be fused in frame to the N-terminus or the C-terminus of any Norrin polypeptide or Norrin mutant polypeptide as disclosed herein. In some embodiments, the Norrin construct includes a Norrin mutant polypeptide of SEQ ID NOs: 2-38 and 60-62, or a an isolated Norrin mutant polypeptide having one or more, or two or more, or one to seven, or two to seven amino acid substitutions at positions: 93, 95, 131, 89, 123, 41, 43, 44, 45, 59, 60, 61, 120, 121, 122, 52, 53, 54, 107, 109, 115, 55, and 110 relative to SEQ ID NO: 1, or having one or more amino acid substitutions at positions 93, 131, 59, 122, 52, 53 107, and 109, or a Norrin construct having one or more amino acid substitutions relative to SEQ ID NO:1 selected from: C93A, C95A, C131A, F89R, R41E, H43A, Y44A, V45A, M59A, L61A, Y120A, R121A, Y122A, L52A, Y53A, K54A, R107E, R109E, and R115.

In some embodiments, a recombinant MBP-Norrin construct can include a recombinant MBP encoding nucleic acid fused in frame to the N-terminus or the C-terminus of any Norrin polypeptide or Norrin mutant polypeptide encoding nucleic acid, wherein the Norrin construct encoding nucleic acid has the N-terminal 72 nucleotide coding portion (i.e. amino acids 1-24) deleted. In some embodiments, the MBP-Norrin construct includes the Norrin construct of SEQ ID NO:1-38 excluding the first (or N-terminal) 24 amino acids MRKHVLAASF SMLSLLVIMG DTDS fused in frame with an MBP protein, for example, a bacterial MBP. In one embodiment, an illustrative MBP-Norrin construct can include a Norrin construct having an amino acid length ranging from 10 to 150 amino acids, in which the amino acid sequence comprises between 10 to 133 contiguous amino acids of SEQ ID NO: 1 and comprises at least one mutation selected from Table 1. In some embodiments, a Norrin construct can include a Norrin mutant polypeptide having an amino acid length ranging from 10 to 133 amino acids, in which the amino acid sequence comprises 10 to 133 contiguous amino acids of SEQ ID NO: 1 and comprises at least one mutation selected from C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, for example, an amino acid substitution of C93A, C131A, M59A, Y122A, L52A, Y53A, and R107E relative to SEQ ID NO: 1. In some embodiments, an isolated Norrin construct comprises the amino acid sequence of SEQ ID NOs: 2, 4, 15, 23, 26, 27 or 33. In some embodiments, a representative MBP-Norrin construct can comprise at least one mutation selected from C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, relative to SEQ ID NO:1. In some embodiments, the fusion protein comprises a Norrin mutant polypeptide fused to a protein selected from maltose binding protein (MBP), glutathione S-transferase (GST) tag, a 6×-His, 8×-His tags and a FLAG-tag among others, wherein the Norrin construct comprises the amino acid sequence of SEQ ID NO:1 having a number of substitutions ranging from one to seven amino acid substitutions, or one to five amino acid substitutions or one to three amino acid substitutions. In one illustrative embodiment, the Norrin construct comprises an isolated Norrin mutant polypeptide having an amino acid sequence in which the one to seven amino acid substitutions, or one to five amino acid substitutions or one to three, or two or more, or two, or three amino acid substitutions occur at positions: 93, 95, 131, 89, 123, 41, 43, 44, 45, 59, 60, 61, 120, 121, 122, 52, 53, 54, 107, 109, 115, 55, and 110 relative to SEQ ID NO:1, or the one to seven amino acid substitutions, or one to five amino acid substitutions or one to three amino acid substitutions occur at amino acid positions: 93, 131, 59, 122, 52, 53 107, and 109 in SEQ ID NO:1.

In some embodiments, the present technology provides a method of synthesizing a recombinant MBP-Norrin construct. In some of these embodiments, the method includes the steps: (a) providing a nucleic acid comprising a nucleic acid sequence encoding a bacterial maltose binding protein (MBP) operatively fused to a nucleic acid sequence encoding a Norrin construct; (b) expressing said nucleic acid in a bacterial strain comprising a gor and a trxB genetic mutation; (c) disrupting the integrity of the bacterial cell wall to provide a crude extract; (d) isolating the MBP-Norrin construct from the crude extract using an amylose affinity column and (e) mixing the isolated MBP-Norrin protein with a shuffling solution comprising arginine, reduced gluthathione, oxidized gluthathione, and a disulfide bond isomerase. As provided above, the method can employ the use of a nucleic acid encoding a Norrin construct as described above, i.e. a Norrin wild-type protein or a Norrin mutant polypeptide as provided herein, for example, a Norrin mutant polypeptide of Table 1.

In illustrative embodiments, the method employs fusion of the MBP to a Norrin construct at either the N-terminus of the Norrin construct, or the C-terminus of the Norrin construct. Similarly, the N-terminus of the MBP can be the N-terminus of the MBP-Norrin construct, or the N-terminus of the MBP can be fused to the N-terminus of the Norrin construct. In some embodiments, the method employs fusion of an E. coli MBP protein.

In some illustrative examples, the method further employs the use of a shuffling solution comprising an equimolar amount of reduced glutathione to oxidized glutathione. In some embodiments, the method can further employ an additional chromatography step after the isolated MBP-Norrin construct is mixed with the shuffling solution. In some embodiments, the additional chromatography step can include re-application of the isolated MBP-Norrin construct on an amylose affinity column or passing the MBP-Norrin construct/shuffling solution mixture through a gel size exclusion chromatography column.

In some embodiments, the method of synthesizing a recombinant MBP-Norrin construct provides an isolated and purified MBP-Norrin construct that has a purity (on a % wt versus protein contaminants) that is typically greater than 90% pure, or greater than 95% pure, or greater than 96% pure, or greater than 97% pure, or greater than 98% pure, or greater than 99% pure, or greater than 99.5% pure, or greater than 99.9% pure.

### C. Recombinant Constructs and Vectors

In certain aspects, the present invention also provides isolated and/or recombinant nucleic acids encoding a Norrin mutant polypeptide or a Norrin fusion protein. The subject nucleic acids may be single-stranded or double-stranded, DNA or RNA molecules. These nucleic acids are useful as therapeutic agents. For example, these nucleic acids are useful in making recombinant Norrin mutant polypeptides which are administered to a cell or an individual as therapeutics. Alternative, these nucleic acids can be directly administered to a cell or an individual as therapeutics such as in gene therapy. In certain embodiments, the invention provides isolated or recombinant nucleic acid sequences that are at least 80%, 85%, 90%, 95%, 97%, 98%, 99% or 100% identical to a region of the nucleotide sequence depicted in SEQ ID NO:39 in which the nucleotide sequence encodes a Norrin mutant polypeptide as described herein. One of ordinary skill in the art will appreciate that nucleic acid sequences complementary to the subject nucleic acids, and variants of the subject nucleic acids are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleotide sequences that hybridize under highly stringent conditions to the nucleotide sequence depicted in SEQ ID NO:39, or a complement sequence thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature. In some embodiments, the recombinant nucleic acids of the invention may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In some embodiments, the nucleotide sequence encoding a Norrin mutant polypeptide is operably fused (in frame) to a different signal peptide other than the first 24 amino acid sequences of SEQ ID NO:1, for example, the Norrin mutant polypeptide lacks the first 24 amino acids, but rather is fused to a maltose binding protein at the N-terminus.

In some embodiments, the subject nucleic acid is provided in an expression vector comprising a nucleotide sequence encoding a Norrin mutant polypeptide and operably linked to at least one regulatory sequence. Regulatory sequences are art-recognized and are selected to direct expression of the soluble polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a soluble polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This invention also pertains to a host cell transfected with a recombinant gene including a coding sequence for one or more of the subject Norrin mutant polypeptides. The host cell may be any prokaryotic or eukaryotic cell. For example, a soluble polypeptide of the invention may be expressed in bacterial cells such as E. coli, insect cells (e.g., using a baculovirus expression system), yeast, or mammalian cells. Other suitable host cells are known to those skilled in the art. Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pETDuet™, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements. In some embodiments of the present invention, transcription of the DNA encoding the wild-type and/or mutant NNO polypeptides by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin by 100 to 270, a cytomegalovirus early promoter enhancer, the polyoma enhancer on the late side of the replication origin, and adenovirus enhancers.

In certain embodiments of the present invention, the DNA sequence in the expression vector is operatively linked to an appropriate expression control sequence(s) (for example, a promoter) to direct mRNA synthesis. Promoters useful in the present invention include, but are not limited to, the LTR or SV40 promoter, the E. coli lac or trp, the phage lambda PL and PR, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or selectable antibiotic markers, for example, tetracycline or ampicillin resistance in E. coli).

In other embodiments, the expression vector may also contain a ribosome binding site for translation initiation (IRES) and a transcription terminator. In still other embodiments of the present invention, the vector may also include appropriate sequences for amplifying expression.

In a further embodiment, the present invention provides host cells containing the above-described vector constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, species within the genera Pseudomonas, Streptomyces, Staphylococcus, as well as eukaryotic host cells Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, C127, 3T3, 293, 293T, HeLa, epithelial cell lines, (for example, A549, BEAS-2B, PtK1, NCI H441), BHK cell lines, T-1 (tobacco cell culture line), root cell and cultured plant cells.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation, gene gun approach and other known methods for introducing DNA into cells (See e.g., Davis et al. [1986] Basic Methods in Molecular Biology). Alternatively, in some embodiments of the present invention, the polypeptides and polynucleotides, including nucleic acid probes of the invention can be synthetically produced by conventional peptide and oligonucleotide synthesizers.

Polypeptides and proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al. (1989) Molecular Cloning: A Laboratory Manual, Second Edition, Cold Spring Harbor, N.Y. Exemplary methods for expressing Norrin, Norrin fusion proteins and Norrin mutant polypeptides are provided in further detail in the Examples below.

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of Norrin proteins can be disrupted by any convenient method, including freeze-thaw cycling, sonication, mechanical disruption, or use of cell lysing agents.

The protein can be expressed in insect cells using baculoviral vectors, or in mammalian cells using vaccinia virus or specialized eukaryotic expression vectors. For expression in mammalian cells, the cDNA sequence may be ligated to heterologous promoters, such as the simian virus (SV 40) promoter in the pSV2 vector or other similar vectors and introduced into cultured eukaryotic cells such as COS cells to achieve transient or long-term expression. The stable integration of the chimeric gene construct may be maintained in mammalian cells by biochemical selection, such as neomycin and mycophenolic acid. The DNA sequence can be altered using procedures such as restriction enzyme digestion, fill-in with DNA polymerase, deletion by exonuclease, extension by terminal deoxynucleotide transferase, ligation of synthetic or cloned DNA sequences and site-directed sequence alteration with the use of specific oligonucleotides together with PCR.

The cDNA sequence or portions thereof can be introduced into eukaryotic expression vectors by conventional techniques. These vectors permit the transcription of the cDNA in eukaryotic cells by providing regulatory sequences that initiate and enhance the transcription of the cDNA and ensure its proper splicing and polyadenylation. The endogenous NNO gene promoter can also be used. Different promoters within vectors have different activities, which alters the level of expression of the cDNA. In addition, certain promoters can also modulate function such as the glucocorticoid-responsive promoter from the mouse mammary tumor virus.

Cell lines can also be produced which have integrated the vector into the genomic DNA. In this manner, the gene product is produced on a continuous basis. Vectors are introduced into recipient cells by various methods including calcium phosphate, strontium phosphate, electroporation, lipofection, DEAE dextran, microinjection, or by protoplast fusion. Alternatively, the cDNA can be introduced by infection using viral vectors. Using the techniques mentioned, the expression vectors containing the NNO gene or portions thereof can be introduced into a variety of mammalian cells from other species or into non-mammalian cells. The recombinant expression vector, according to this invention, comprises the selected DNA of the DNA sequences of this invention for expression in a suitable host. The DNA is operatively joined in the vector to an expression control sequence in the recombinant DNA molecule so that normal or mutant protein can be expressed. The expression control sequence may be selected from the group consisting of sequences that control the expression of genes of prokaryotic or eukaryotic cells and their viruses and combinations thereof. The expression control sequence may be selected from the group consisting of the lac system, the trp system, the tac system, the trc system, major operator and promoter regions of phage lambda, the control region of the fd coat protein, early and late promoters of SV40, promoters derived from polyoma, adenovirus, retrovirus, baculovirus, simian virus, 3-phosphoglycerate kinase promoter, yeast acid phosphatase promoters, yeast alpha-mating factors and combinations thereof.

The host cells to be transfected with the vectors of this invention may be from a host selected from the group consisting of yeasts, fungi, insects, mice or other animals or plant hosts or may be human tissue cells. For the mutant DNA sequence, similar systems are employed to express and produce the Norrin mutant polypeptides.

### D. Pharmaceutical Compositions

The invention further contemplates pharmaceutical compositions comprising a Norrin mutant polypeptide. In one embodiment, exemplary pharmaceutical compositions include pharmaceutical compositions comprising a Norrin mutant polypeptide, formulated in a pharmaceutically acceptable carrier or excipient. In other embodiments, pharmaceutical compositions include pharmaceutical compositions comprising a Norrin polypeptide of the present invention fused to maltose binding protein, for example, a maltose binding protein as provided in Table 1. Further exemplary pharmaceutical compositions include pharmaceutical compositions comprising one or more Norrin mutant polypeptides, and a secondary anti-angiogenesis agent. Such secondary agents include, but are not limited to, anti-angiogenic agents, for example, a VEGF antagonist or inhibitor, for example, an anti-VEGF antibody or active fragment thereof (e.g., humanized A4.6.1, Avastin®) RNA aptamers, and ribozymes against VEGF or VEGF receptors or small molecules that block VEGF receptor signaling (e.g., PTK787/ZK2284, SU6668). Anti-angiogenesis agents also include native angiogenesis inhibitors, e.g., angiostatin, endostatin, etc. See, e.g., Klagsbrun and D'Amore, Ann. Rev. Physiol., 53:217-39 (1991); Streit and Detmar, Oncogene, 22:3172-3179 (2003) (e.g., Table 3 of Streit et al. listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 of Tonini et al. listing anti-angiogenic factors); and, Sato, et al., Int. J. Clin. Oncol., 8:200-206 (2003) (e.g., Table 1 of Sato et al., which lists anti-angiogenic agents used in clinical trials), the contents of these disclosures are incorporated herein by reference in their entirety.

### E. Formulations and Administration

In some embodiments, the present invention discloses methods for preparing pharmaceutical compositions comprising a Norrin mutant polypeptide or a Norrin fusion protein as an active ingredient. In the pharmaceutical compositions and methods of the present invention, the active ingredient will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, sprays, liquid drops, washes, ointments topical liposome formulations and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture.

Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below. Additionally, the compositions of the present invention may be formulated in sustained release form to provide the rate controlled release of any one or more of the components or active ingredients to optimize the therapeutic effects, i.e. anti-angiogenic activity and the like. Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen. Also included are solid form preparations which are intended to be converted, shortly before use, to liquid form preparations for either oral or parenteral administration. Such liquid forms include solutions, suspensions and emulsions. In some embodiments, pharmaceutical compositions may be formulated as liposomes.

The Norrin mutant polypeptide may also be deliverable transdermally. The transdermal compositions may take the form of creams, lotions, aerosols and/or emulsions and can be included in a transdermal patch of the matrix or reservoir type as are conventional in the art for this purpose. The choice of formulation depends on various factors such as the mode of drug administration (e.g., for intraocular injection or intratumoral injection, such compositions may be formulated as liquids e.g., solutions, oil in water and/or water in oil emulsions or gels) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants. One specific route of administration is oral, using a convenient daily dosage regimen that can be adjusted according to the degree of severity of the disease-state to be treated.

Solid dosage forms for oral administration include capsules, tablets, pills, powders, and granules, preferably formulated with enteric coatings or encapsulated acid resistant/tolerable polymers to preserve the biological activity of the polypeptides of the present invention. In such solid dosage forms, the Norrin mutant polypeptide is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the Norrin mutant polypeptide in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a Norrin mutant polypeptide, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3 butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. In some embodiments, the compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, and physiologically acceptable lipid capable of forming liposomes can be used. The compositions in liposome form may contain stabilizers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

Dosage forms for topical administration of a Norrin mutant polypeptide include ointments, powders, sprays, and inhalants. The active component is admixed under sterile conditions with a physiologically acceptable carrier and any preservatives, buffers, or propellants as may be required. Compressed gases may be used to disperse a Norrin mutant polypeptide of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a Norrin mutant polypeptide of the present invention, and 99% to 1% by weight of one or more suitable pharmaceutical excipients. In one example, the composition will be between about 5% and about 75% by weight of a Norrin mutant polypeptide of the invention, with the rest being one or more suitable pharmaceutical excipients. If formulated as a fixed dose, in some embodiments, such products employ the Norrin mutant polypeptide of this invention within the dosage range described above and optionally, a secondary active agent(s) within its approved therapeutic dosage range. In one embodiment, Norrin mutant polypeptide and secondary agents of the instant invention may alternatively be used sequentially with known pharmaceutically acceptable agent(s) when a combination formulation is inappropriate.

In some embodiments, a Norrin mutant polypeptide of the invention, can be administered in a therapeutically effective amount which will vary depending upon a variety of factors including the activity of the specific polypeptide employed, the metabolic stability and length of action of the polypeptide, the age, body weight, general health, sex, diet, mode and time of administration, rate of excretion, drug combination, the severity of the particular disease-states, and the host undergoing therapy. Preferably, the pharmaceutical preparation is in a unit dosage form. In such form, the preparation is subdivided into suitably sized unit doses containing appropriate quantities of the active components, e.g., a therapeutically effective amount of a Norrin mutant polypeptide to achieve the desired purpose, for example, reduction in Norrin-Fz4 mediated signaling leading to a reduction in angiogenesis. In some embodiments, a pharmaceutical composition comprises a quantity of the Norrin mutant polypeptide formulated in a unit dose form which may be generally varied or adjusted from about 0.1 milligram to about 1,000 milligrams, preferably from about 1 to about 750 milligrams, more preferably from about 5 to about 500 milligrams, and typically from about 1 to about 250 milligrams, according to the particular application. Each unit dose may form a daily dose or may be a partial dose of a daily dose. Daily doses may range from about 1-10,000 mg per day or more, or about 1-2,000 mg per day or more, or about 1-1,000 mg per day or more, or about 1-500 mg per day or more, or about 1-100 mg or more. In some embodiments, a pharmaceutical composition comprises a therapeutically effective amount of a Norrin mutant polypeptide, or a Norrin polypeptide as recited in Table 1, and a pharmaceutically acceptable carrier, vehicle or excipient. In some embodiments, the present invention provides a pharmaceutical composition comprising a therapeutically effective amount of a Norrin mutant polypeptide comprising an amino acid sequence of SEQ ID NOs: 2-38 and 60-62, in admixture with at least one pharmaceutically acceptable carrier, vehicle or excipient. In some embodiments, the present invention provides a pharmaceutical composition comprising a therapeutically effective amount of a Norrin fusion protein comprising an amino acid sequence of SEQ ID NO: 40, in admixture with at least one pharmaceutically acceptable carrier, vehicle or excipient.

In another embodiment, the present invention provides a pharmaceutical composition comprising a therapeutically effective amount of a Norrin mutant polypeptide comprising an amino acid sequence of SEQ ID NOs: 2-38 and 60-62, for example, SEQ ID NO: 2, 4, 15, 23, 26, 27 or 33, in admixture with at least one pharmaceutically acceptable carrier, vehicle or excipient. In some embodiments, the present invention provides a pharmaceutical composition comprising a therapeutically effective amount of a Norrin mutant polypeptide, the Norrin mutant polypeptide comprising the amino acid sequence of SEQ ID NO: 1, said sequence having one or more amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1, for example, at positions 131, 59, 122, 52, 53, 93, 107, and 109 relative to SEQ ID NO:1. In accordance with the present invention, in one aspect, the invention provides a pharmaceutical composition comprising a therapeutically effective amount of a Norrin mutant polypeptide, wherein the polypeptide has two to seven, two to five, two to four, one to three, three, two, or one amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1, for example, at positions 131, 59, 122, 52, 53, 93, 107, and 109 relative to SEQ ID NO:1.

In one embodiment, a pharmaceutical composition comprises a therapeutically effective amount of a Norrin mutant polypeptide as disclosed herein. In related embodiments, a pharmaceutical composition comprises a therapeutically effective amount of a Norrin mutant polypeptide, wherein the polypeptide has an amino acid sequence of SEQ ID NO:1, having an amino acid substitution at one or more of amino acids: 93, 131, 59, 122, 52, 53, and 107 of SEQ ID NO:1 in admixture with at least one pharmaceutically acceptable carrier, vehicle or excipient. For example, an isolated Norrin mutant polypeptide of the present pharmaceutical compositions can have an amino acid substitution is selected from the group consisting of C93A, C131A, M59A, Y122A, L52A, Y53A, and R107E. In another aspect, the present invention provides a pharmaceutical composition comprising a therapeutically effective amount of a Norrin mutant polypeptide, the polypeptide comprising an amino acid sequence of SEQ ID NOs: 2-38 and 60-62, for example, an amino acid sequence of SEQ ID NO:2, 4, 15, 23, 26, 27 or 33 in admixture with at least one pharmaceutically acceptable carrier, vehicle or excipient.

In accordance with the present invention, the present invention provides a pharmaceutical composition comprising a therapeutically effective amount of a Norrin mutant polypeptide, wherein the polypeptide has an amino acid sequence having one or more amino acid substitutions as shown in Table 1 in admixture with at least one pharmaceutically acceptable carrier, vehicle or excipient. In one such example, the isolated Norrin mutant polypeptide has an amino acid sequence having one or more amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. For example, in one embodiment, the isolated Norrin mutant polypeptide has an amino acid sequence having two or more amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another example, the isolated Norrin mutant polypeptide has an amino acid sequence having two to seven amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or two to five amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or one, two or three amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E.

In some embodiments, the present invention provides a pharmaceutical composition comprising a therapeutically effective amount of a Norrin mutant polypeptide, wherein the polypeptide has an amino acid sequence having one to seven amino acid substitutions relative to SEQ ID NO:1 comprising: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E in admixture with at least one pharmaceutically acceptable carrier, vehicle or excipient. In various aspects, an exemplary isolated Norrin mutant polypeptide has an amino acid substitution as defined above, wherein the amino acid substitution or substitutions is or are, conservative amino acid substitutions.

In some embodiments, the present invention provides a pharmaceutical composition comprising a therapeutically effective amount of a Norrin mutant polypeptide, wherein the polypeptide comprises the amino acid sequence of SEQ ID NO: 1, wherein the amino acid sequence of SEQ ID NO:1 has one or more, or one to seven, or one to five, or one, two or three amino acid substitutions at positions 93, 131, 59, 122, 52, 53 and 107 relative to SEQ ID NO:1 in admixture with at least one pharmaceutically acceptable carrier, vehicle or excipient. In some embodiments, the present invention provides a pharmaceutical composition comprising a therapeutically effective amount of a Norrin mutant polypeptide comprising one or more mutations selected from: C93A, C131A, M59A, Y122A, L52A, Y53A, and R107E, relative to SEQ ID NO:1 in admixture with at least one pharmaceutically acceptable carrier, vehicle or excipient.

Generally, the human oral dosage form containing the active ingredients can be administered from 1 to 5 times per day. The amount and frequency of the administration will be regulated according to the judgment of the attending clinician. The Norrin mutant polypeptide of the present invention can be administered to a patient at dosage levels in the range of about 0.01 to about 10,000 mg per day in single or divided doses. For a normal human adult having a body weight of about 70 kilograms, a dosage in the range of about 0.01 to about 100 mg per kilogram of body weight per day, or more preferably from about 0.05 to about 10 mg per kilogram per day, or from about 0.1 to about 5 mg per kilogram of body weight is an example. The specific dosage used, however, can vary. The determination of optimum dosages for a particular patient is well known to one of ordinary skill in the art.

For ophthalmic compositions, typically, the compositions are administered as drops or an ophthalmic solution for intraocular administration, with the composition being applied to an eye of the subject suffering from or susceptible to a disease associated with hypervascularization or neovascularization, although more or less of the composition may be used in more or less frequent doses depending on multiple factors, including the makeup of the particular composition. In some embodiments, the compositions of the present invention are administered intraocularly, directly into contact with the retina of the subject to be treated. In some embodiments, the compositions of the present invention are administered using stereo tactic guidance using an assistive imaging technology operable to identify the location of a solid tumor mass. The composition can then be guided using a laparoscopic device, or a stent and the like, and injected intratumorally into the tumor or in sufficient proximity to contact the tumor and surrounding tissue and blood vessels.

Typically, in therapeutic applications, the treatment would be for the duration of the disease state or condition, or may be temporally administered to inhibit, reduce or prevent angiogenesis in the required tissue. Further, it will be apparent to one of ordinary skill in the art that the optimal quantity and spacing of individual dosages will be determined by the nature and extent of the disease state or condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests. The administration of the pharmaceutical compositions above can be repeated several times, preferably at least one to five times, in daily, weekly, or monthly intervals. In some embodiments, a unit dose may be administered one to three times per day or once per day in sustained release form to reduce or inhibit angiogenesis, for example, while the patient is receiving other ophthalmic treatments. The frequency of dosing can be experimentally verified in clinical trials and are recommended to provide a reasonable benefit/risk ratio commensurate with the experience of the prescribing clinician. Methods for determining the therapeutic effectiveness of the compositions described herein for the treatment of various allergic diseases or conditions are well within the skill of the ordinary artisan.

For any of the foregoing, the invention contemplates administration to neonatal, infant, children, adolescent, and adult patients, and one of skill in the art can readily adapt the methods of administration and dosage described herein based on the age, health, size, and particular disease status of the patient. Furthermore, the invention contemplates administration in utero to treat conditions in an affected fetus, particularly those with a tumor or neovascularization or hypervascularization of the eye, including the retina. The actual dosage employed may be varied depending upon the patient's age, sex, weight and severity of the condition being treated. Such techniques are well known to those skilled in the art. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a Norrin mutant polypeptide of the invention, for treatment of a disease-state in accordance with the teachings of this invention.

### Ophthalmic Compositions

Ophthalmic formulations including eye ointments, powders, sprays, liquid drops, washes, ointments, topical liposome formulations are also contemplated as being within the scope of this invention. As used herein, “concentration” of a component of an ophthalmic composition means concentration based on mass of the component per total volume of the composition (i.e., g/mL, or wt/vol), and is typically expressed as a percentage.

In some embodiments, an ophthalmic composition can include a therapeutically effective amount of a Norrin mutant polypeptide, ranging from about 0.01% to about 10% (w/v), or from about 0.02% to about 0.1% (w/v) in admixture with a suitable ophthalmic carrier. In some embodiments, an ophthalmic composition for topical application or intraocular injection can include a therapeutically effective concentration of a Norrin mutant polypeptide comprising an amino acid sequence of SEQ ID NOs: 2-38 and 60-62 ranging from about 0.01% to about 1.0% (wt/vol). In some embodiments, for topical ophthalmic administration, the ophthalmic carrier can include: water, mixtures of water and water-miscible solvents, such as C1- to C7-alkanols, vegetable oils or mineral oils comprising from 0.5 to 5 percent by weight ethyl oleate, hydroxyethylcellulose, carboxymethylcellulose, polyvinylpyrrolidone and other non-toxic water-soluble polymers for ophthalmic uses, may include, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenan, agar and acacia, starch derivatives, such as starch acetate and hydroxypropyl starch, and also other synthetic products, such as polyvinyl alcohol, polyvinyl pyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, salts of carboxymethylcellulose, hydroxymethylcellulose, hydroxyethylcellulose, methylhydroxypropylcellulose and hydroxypropylcellulose, neutral Carbopol, or mixtures thereof. A highly preferred carrier is water. The concentration of the carrier is, for example, from 1 to 100,000 times the concentration of the active ingredient.

In some embodiments, the ophthalmic composition for topical or intra-ocular administration may optionally also include a non-ionic tonicity agent. In some embodiments, a non-ionic tonicity agent includes glycerol, although other non-ionic tonicity agents may be used such as, for example, urea, sorbitol, mannitol, propylene glycol, and dextrose. In some embodiments, the non-ionic tonicity agent is provided in a concentration such that the composition has an osmolality from 400 to 750 milliosmoles/kilogram (mOsm/Kg), preferably from 425 to 700 mOsm/Kg, more preferably from 550 to 700 mOsm/Kg, even more preferably from 600 to 700 mOsm/Kg, and yet even more preferably from 650 to 700 mOsm/Kg. In some embodiments, glycerol is used as the non-ionic tonicity agent in a concentration of from 3% to 10%, preferably from 4% to 8%, more preferably from 5% to 7%, even more preferably from 5.5. % to 6.5%, and yet even more preferably from 5.75% to 6.25%. In yet other embodiments, glycerol is used as the non-ionic tonicity agent in a concentration of greater than 3.5%, preferably greater than 4.5%, more preferably greater than 5.5%, even more preferably from 5% to 7%, and yet even more preferably from 5.5% to 6.5%, such that the composition has an osmolality from 400 to 750 mOsm/Kg, preferably from 425 to 700 mOsm/Kg, more preferably from 550 to 700 mOsm/Kg, even more preferably from 600 to 700 mOsm/Kg, and yet even more preferably from 650 to 700 mOsm/Kg.

The ophthalmic compositions of the present invention may optionally also include one or more preservatives, particularly when the composition is packaged as a multi-dose application. Illustrative preservatives can include: benzalkonium chloride, polyquad preservative (Alcon); perborate (e.g., sodium perborate from Ciba); purite preservative (stabilized chlorine dioxide) (Allergan); other quaternary ammonium compounds such as benzoxonium chloride; alkyl-mercury salts of thiosalicylic acid such as, for example, thiomersal, phenylmercuric nitrate, phenylmercuric acetate, and phenylmercuric borate; parabens such as, for example, methylparaben or propylparaben; alcohols such as, for example, chlorobutanol, benzyl alcohol, and phenyl ethanol; guanidine derivatives such as, for example, chlorhexidine or polyhexamethylene biguanide; and the like. When a preservative is used in the ophthalmic composition, the preservative is typically provided in a concentration of 0.005% to 0.02%, preferably 0.01%, although other concentrations may be used.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular, intraperitoneal, intraocular, stereotactically, intratumorally, or intravenous injection. Typically, dosages of the compound of the invention which may be administered to an animal, preferably a human, will vary depending upon any number of factors, including but not limited to, the type of animal and type of disease state being treated, the age of the animal and the route of administration.

Pharmaceutical compositions of the invention formulated for pulmonary delivery may also provide the active ingredient or active ingredients, in the form of droplets of a solution or suspension. Such formulations may be prepared, packaged, or sold as aqueous or dilute alcoholic solutions or suspensions, optionally sterile, comprising the active ingredient, or active ingredients and may conveniently be administered using any nebulization or atomization device. Such formulations may further comprise one or more additional non-active ingredients for example, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers. The formulations described herein as being useful for pulmonary delivery are also useful for intranasal delivery of a pharmaceutical composition of the invention.

In some embodiments, an exemplary formulation suitable for intranasal administration is a coarse powder comprising the active ingredient and having an average particle size or diameter from about 0.2 to 500 micrometers. Such a formulation can be administered by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 50% (w/w) active ingredient, (and ranges inherent therein) the balance comprising an orally dissolvable or degradable composition and, optionally, one or more of the additional non-active ingredients described herein. Alternately, formulations suitable for buccal administration may comprise a powder or an aerosolized or atomized solution or suspension comprising the active ingredient or active ingredients. Such powdered, aerosolized, or aerosolized formulations, when dispersed, preferably have an average particle or droplet size in the range from about 0.1 to about 200 nanometers, and may further comprise one or more of the additional non-active ingredients described herein.

As used herein, “additional non-active ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional non-active ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

### F. Kits

The present invention also provides a kit comprising a composition of the invention and a delivery device. The compositions may conveniently be presented in single or multiple unit dosage forms as well as in bulk, and may be prepared by any of the methods which are well known in the art of pharmacy. The composition, found in the kit, whether already formulated together or where the compounds are separately provided along with other ingredients, and instructions for its formulation and administration regime. The kit may also contain other agents, such as those described elsewhere herein and, for example, when for parenteral administration, they may be provided with a carrier in a separate container, where the carrier may be sterile. The present composition may also be provided in lyophilized form, and in a separate container, which may be sterile, for addition of a liquid carrier prior to administration. In a specific embodiment, the kit of the present invention comprises a Norrin mutant polypeptide of the invention, an applicator, and an instructional material for the use thereof. In another embodiment, the kit can comprise a Norrin mutant polypeptide, such as those described elsewhere herein, a container holding the polypeptide, and an instructional material. The skilled artisan can provide the applicator.

Preferably, the kit of the present invention comprises a Norrin mutant polypeptide, or a Norrin mutant polypeptide of Table 1, or combinations thereof. In some embodiments, the Norrin mutant polypeptide the Norrin mutant polypeptide comprising the amino acid sequence of SEQ ID NO: 1, said sequence having one or more amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1, for example, at positions 131, 59, 122, 52, 53, 93, 107, and 109 relative to SEQ ID NO:1. In accordance with the present invention, in one aspect, the invention provides a kit of the present invention comprising a therapeutically effective amount of a Norrin mutant polypeptide, wherein the polypeptide has two to seven, two to five, two to four, one to three, three, two, or one amino acid substitutions at positions 93, 95, 131, 89, 123, 41, 43, 44, 45, 59, 60, 61, 120, 121, 122, 52, 53, 54, 107, 109, 115, 55, and 110 relative to SEQ ID NO: 1, for example, at positions 131, 59, 122, 52, 53, 93, 107, and 109 relative to SEQ ID NO:1.

In one embodiment, a kit of the present invention comprises a therapeutically effective amount of a Norrin mutant polypeptide as disclosed herein. In related embodiments, a kit of the present invention comprises a therapeutically effective amount of a Norrin mutant polypeptide, wherein the polypeptide has an amino acid sequence of SEQ ID NO:1, having an amino acid substitution at one or more of amino acids: 93, 131, 59, 122, 52, 53, and 107 of SEQ ID NO:1 optionally in admixture with at least one pharmaceutically acceptable carrier, vehicle or excipient. For example, an isolated Norrin mutant polypeptide of the present kit can have an amino acid substitution is selected from the group consisting of C93A, C131A, M59A, Y122A, L52A, Y53A, and R107E. In another aspect, the present invention provides a kit comprising a therapeutically effective amount of a Norrin mutant polypeptide, the polypeptide comprising an amino acid sequence of SEQ ID NOs: 2-38 and 60-62, for example, an amino acid sequence of SEQ ID NO:2, 4, 15, 23, 26, 27 or 33 in admixture with at least one pharmaceutically acceptable carrier, vehicle or excipient.

In accordance with the present invention, the present invention provides a kit comprising a therapeutically effective amount of a Norrin mutant polypeptide, wherein the polypeptide has an amino acid sequence having one or more amino acid substitutions as shown in Table 1 optionally in admixture with at least one pharmaceutically acceptable carrier, vehicle or excipient. In one such example, the isolated Norrin mutant polypeptide has an amino acid sequence having one or more amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. For example, in one embodiment, the isolated Norrin mutant polypeptide has an amino acid sequence having two or more amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E. In another example, the isolated Norrin mutant polypeptide has an amino acid sequence having two to seven amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or two to five amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, or one, two or three amino acid substitutions relative to SEQ ID NO:1 selected from the group consisting of: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E.

In some embodiments, the present invention provides a kit comprising a therapeutically effective amount of a Norrin mutant polypeptide, wherein the polypeptide has an amino acid sequence having one to seven amino acid substitutions relative to SEQ ID NO:1 comprising: C93A, C95A, C131A, C55A, C110A, F89R, R41E, H43A, Y44A, V45A, M59A, V60A, L61A, Y120A, R121A, Y122A, L52A, Y53A, K54A, K54E, R107E, R109E, and R115E, optionally in admixture with at least one pharmaceutically acceptable carrier, vehicle or excipient. In various aspects, an exemplary isolated Norrin mutant polypeptide has an amino acid substitution as defined above, wherein the amino acid substitution or substitutions is or are, conservative amino acid substitutions.

Additionally, the kit can comprise an instructional material and an applicator for the administration of the compound(s) of the present invention for the treatment of a disease or condition associated with aberrant angiogenesis. The kits of the present invention can be used to treat the diseases and conditions disclosed herein. The kits described in the present invention are not limited to the uses above however, and can be used in any method derived from the teachings disclosed herein.

### G. Methods of Making Recombinant Norrin

In some embodiments, the present invention provides a robust and reproducible method for synthesizing, isolating and purifying a Norrin construct (i.e. a wild-type Norrin, Norrin mutant polypeptides, and Norrin fusion proteins) in high quantities and purity. In one embodiment, the method includes the steps:

a. providing a nucleic acid comprising a nucleic acid sequence encoding a bacterial maltose binding protein (MBP) operatively fused to a nucleic acid sequence encoding a mature Norrin polypeptide, for example, as shown in SEQ ID NO:43. b. expressing said nucleic acid in a bacterial strain comprising a gor and a trxB genetic mutation; c. disrupting the integrity of the bacterial cell wall to provide a crude extract; d. isolating the MBP-Norrin construct from the crude extract using an amylose affinity column; and e. mixing the isolated MBP-Norrin protein with a shuffling solution comprising arginine, reduced gluthathione, oxidized gluthathione, and a disulfide bond isomerase. In some embodiments, the MBP-Norrin protein has a thrombin cleavage site engineered between the C-terminus of the MBP protein and the N-terminus of the mature Norrin amino acid sequence. Optionally, step (e) or step (f) comprises removing the MBP terminal protein from the MBP-Norrin construct, for example with thrombin. In some embodiments, step a. includes providing a nucleic acid comprising a nucleic acid sequence encoding a bacterial maltose binding protein (MBP) operatively fused to a nucleic acid sequence encoding a Norrin mature polypeptide as shown in SEQ ID NO:41 having one or more mutations in the amino acid sequence as shown in SEQ ID NO: 2-38 and 60-62. In some embodiments, a MBP nucleic acid of SEQ ID NO:44 (for example a MBP protein having an amino acid sequence of SEQ ID NO:42) is operatively in frame with a mature Norrin polypeptide encoding nucleic acid of SEQ ID NO:39.

In some embodiments, the nucleic acid encodes a Norrin construct which may include a Norrin wild-type protein or a Norrin mutant polypeptide. In some embodiments, the method can be used to synthesize recombinantly any Norrin mutant polypeptide described herein, for example, a Norrin mutant polypeptide comprising an amino acid sequence of SEQ ID NOs: 2-38 and 60-62, a Norrin mutant polypeptide comprising at least a portion of SEQ ID NO:1, wherein the polypeptide comprises an amino acid substitution at one or more amino acids comprising amino acids: 93, 131, 59, 122, 52 53 or 107 in SEQ ID No:1, or a Norrin mutant polypeptide comprising one or more mutations selected from: M59A, Y122A, L52A, Y53A, or R107E, relative to SEQ ID NO:1. In various embodiments, the synthesized Norrin polypeptide can include human Norrin as provided in SEQ ID NO: 1, using the DNA as recited in SEQ ID NO: 39, or Norrin derived from a mouse (for example, as provided in NCBI Accession No. NP_{—}035013.1 GI:6754808), from a rat, (for example, as provided in NCBI Accession No. NP_{—}001102284.1 GI:157818563), and from any other species that have the nucleotide (i.e. an mRNA or cDNA) or amino acid sequence that are readily available from the NCBI protein and nucleotide databases.

In some embodiments of the present methods, a bacterial expression vector is created using standard molecular biology techniques. The bacterial expression vector is engineered to contain a first nucleic acid encoding a bacterial maltose binding protein operably in frame with a nucleic acid encoding a Norrin construct (either wild-type or a mutated sequence as provided herein). The vector is then transformed or transfected inserted into a bacterial strain harboring a double mutation in thioredoxin reductase (trxB) and a mutation in glutathione reductase (gor) that promote disulfide bond formation. In one embodiment, the bacterial strain is E. coli Origami (DE) (commercially available from Novagen (EMD Millipore) under cat. no. 71146 pETDuet™-1 DNA).

In various embodiments, the trxB gor bacterial strain is then transformed or transfected with the expression vector containing an in frame fusion protein comprising MBP and a Norrin construct.

In various embodiments, the expression vector can be introduced into the bacterial strain using any method commonly used for bacterial transformations, including chemical and pulse field electroporation methods know to those of skill in the art. The bacterial strain harboring the expression vector can then be grown in any suitable medium to mid log phase and induced with an appropriate inducing agent, for example, IPTG.

Bacterial cells thus transformed can then be disrupted to extract the contents. In some embodiments, cell disruption can include chemical lysis of the bacterial cells, or they may be physically disrupted, for example with the use of homogenization or sonication to yield a crude extract. In various embodiments, the MBP-Norrin construct is released and the crude extract can be further treated to isolate the MBP-Norrin construct. In some embodiments, the crude extract can then be clarified using centrifugation and passed over a maltose affinity column to isolate the MBP-Norrin construct fusion. The samples containing the MBP-Norrin construct fusion is lastly treated with a shuffling solution to encourage disulfide shuffling and dimer formation of the MBP-Norrin construct fusion. The fractions containing the MBP-Norrin construct are mixed with a solution containing oxidized glutathione, reduced glutathione, arginine, and a prokaryotic disulfide bond isomerase (for example, DsbC). In some embodiments, the ratio of oxidized glutathione to reduced glutathione is one to one. Lastly, the MBP-Norrin construct is isolated as a dimer from the rest of the components of the shuffling solution by passing the shuffling solution through a size exclusion chromatography column, (for example, a Sepharose column). Optionally, the shuffling solution can be passed over a Ni²⁺-chelating column prior to size exclusion to remove the prokaryotic disulfide bond isomerase.

In some embodiments, the present invention provides recombinant Norrin fusion proteins. In one embodiment, the fusion protein comprises an isolated Norrin fusion protein, the fusion protein comprising an amino acid sequence of SEQ ID NO:40. In other embodiments, the fusion protein comprises an amino acid sequence of SEQ ID NO:41 having one or more mutations as provided in Table 1, fused at the N-terminus with the C-terminus of a maltose binding protein derived from plant, bacteria or yeast, for example, the maltose binding protein of SEQ ID NO: 42. In some embodiments, the Norrin fusion protein consists of the amino acid sequence of SEQ ID NO:40.

### H. Method for Treating Aberrant Angiogenesis

The present invention also provides methods and compositions comprising agents that inhibit the activity of Norrin mediated signaling and angiogenesis associated therewith, or that inhibit Norrin signaling. Aberrant angiogenesis is a term that encompasses the reduction in angiogenic stimuli as a result of Norrin mediated signaling through β-catenin/TCF activation. Such compositions can be used to inhibit the proliferation, migration, and adhesion of endothelial cells in tissues undergoing aberrant angiogenesis. Such compositions can also be used to inhibit binding of wild-type Norrin to Fz4 and/or Lrp5/6 that directly or indirectly inhibits Wnt signaling. Without wishing to be bound by any particular theory or mechanism, it is believed that competition between wild-type Norrin and the Norrin mutant polypeptides of the present invention inhibits the assembly and activation of the Norrin-Fz4-Lrp5/6-Tspan12 signaling complex leading to a reduction in β-catenin/TCF activation. The reduction in Norrin-Fz4 mediated signaling is believed to confer anti-angiogenic effects in vitro and in vivo.

Therefore, the present compositions find utility in reducing or inhibiting angiogenesis associated with aberrant angiogenic conditions such as hypervascularization of the retina, and angiogenesis associated with tumor growth and metastases as a result of Norrin-Fz4-Lrp5/6-Tspan12 signaling. Exemplified pharmaceutical compositions comprising a Norrin mutant polypeptide with one or more of these activities, can be useful in the inhibition, reduction of aberrant angiogenesis and therefore may benefit a number of exemplary conditions described more fully below.

### I. Ophthalmic Diseases

In various embodiments, the present compositions can be used to primarily reduce or inhibit angiogenesis in a subject having an ophthalmic disease associated with aberrant angiogenesis. In some embodiments, the ophthalmic disease may result from aberrant angiogenesis related to an ocular disease. Ocular neovascularization is believed to be, at least in part, involved in the pathophysiology of several ophthalmic diseases. In some embodiments, the ophthalmic disease can include: diabetic retinopathy, choroidal neovascularization, age-related macular degeneration, hypertensive retinopathy, retinopathy of prematurity, branched central retinal vein occlusion, central retinal vein occlusion, pathological myopia, diabetic macular edema, von Hippel-Lindau disease, and corneal neovascularization.

Retinopathy: Based in part on the anti-angiogenic properties of the present compositions, the methods and compositions of the present invention can be used in the treatment of hypervascular retinopathies. Briefly, described below are two sub-classes of retinopathy: diabetic retinopathy and retinopathy of prematurity (ROP).

Among the more than 10 million people in the United States who have or will develop diabetes, over half will ultimately have some degree of visual loss. Such visual loss is caused in large part by retinopathy.

A cascade of subtle changes that occur in the blood vessel walls, the blood itself, and the very special structures in the retina lead to swelling of the central retinal tissue (macular edema) that blurs the vision of millions of diabetics. More severe prolonged abnormalities will lead to development of abnormal weak blood vessels that can rupture or be the scaffold for scar tissue. Dense blood clots in the central cavity (vitreous gel) of the eye or retinal detachment from traction of scar tissue can lead to profound visual loss or total blindness. Significant retinal changes can occur before any visual changes are noted by the patient. Blurring of vision, increased trouble with glare and an onset of “floaters” may be evidence of beginning visual problems.

Examination for retinopathy includes basic tests of visual acuity, eye pressure (to rule out glaucoma), and an exam through a dilated pupil to see both panoramic and high magnification views of the retina. In addition to the commonly performed fluorescein angiography test that identifies both early and late blood vessel changes by their special forms of excessive leakage, macular tissue damage can be measured by a special electroretinogram (ERG) (principle similar to the electrocardiogram), small central blind spot changes by the scanning laser ophthalmoscope (SLO), hidden changes in a blood filled eye by a sonar-like ultra-sound echo system, and subtle circulatory changes in the retinal blood vessels with the Laser Doppler Flow meter (LDF).

The methods and compositions of the present invention can be used in the treatment of retinopathy, for example in the treatment of diabetic retinopathy. Such methods and compositions can be used alone or in combination with other recognized therapies for retinopathy. Such therapies can include laser photocoagulation and closed vitrectomy. Useful adjunct therapies may also include management of diabetes, for example, methods of stabilizing one's blood glucose and thereby avoiding frequent hyper- and hypo-glycemic states.

Laser treatment is more common than vitrectomy. It is done in an office, with the patient sitting in front of a laser machine. The eye is numbed by anesthesia drops to allow a special contact lens to be placed on the eye to deliver the laser beam. The beam can be changed to minimize discomfort while delivering sufficient energy to create the desired retinal reaction. The laser treatment is performed either to decrease the macular swelling or to reduce the risk of bleeding from abnormal, weak blood vessels.

Retinopathy of Prematurity (ROP) is a disease of the retina, the light sensitive membrane covering the inside of the eye. It affects small, prematurely born babies. It consists of abnormal retinal vessels that grow mostly in an area where normal vessels have not yet grown in the retina. ROP is divided into stages 1 to 5. Stages 1 and 2 do not usually require treatment. Some babies who have developed stage 3 ROP require treatment usually involving laser or cryotherapy.

Peripheral retinal treatment can reduce, but not eliminate, the chance of the ROP progressing to the potentially blinding stages 4 and 5. When stage 4 or 5 ROP is reached, the retina is detached and other therapies can be performed. One such therapy is scleral buckling, which involves encircling the eyeball with a silicone band to try and reduce the pulling on the retina. Other therapies include vitrectomy (removal of the gel-like substance called the vitreous that fills the back of the eye). Sometimes the removal of the lens as well is required during vitrectomy to try and eliminate as much pulling as possible from the retinal surface. Removal of the lens is performed if the retina is touching the back surface of the lens.

In any of the above methods for treating an ophthalmic disease, a secondary active agent may be added to the treatment methods described herein. In some embodiments, a combined treatment of a composition comprising one or more Norrin mutant polypeptides can be administered along with a composition comprising an angiogenesis inhibitory agent. In some embodiments, an angiogenesis inhibitory agent can include: an antagonist or inhibitor of VEGF, angiostatin, or endostatin.

### J. Cancer

Cancer is a catch-all phrase that refers to any of a number of hyper-proliferation conditions affecting nearly every tissue. For example, cancers of the breast, colon, prostate, ovary, testicles, cervix, esophagus, pancreas, bone, lung, brain, skin, liver, stomach, and tongue are well known. Further well known examples of cancers include cancers of the blood such as leukemias and lymphomas.

The dangers posed by cancers are two-fold. First, cancer in a particular tissue may grow, thereby inhibiting the normal function of a particular organ or tissue. Second, cancer may metastasize to other parts of the body, thereby inhibiting the normal function of multiple organs and tissues.

One currently recognized method for treating or otherwise inhibiting the progression of cancer is based on the concept of anti-angiogenesis. Without being bound by theory, the inhibition of angiogenesis prevents tumor growth and survival by depriving those cells of the blood, oxygen, and nutrients necessary to maintain cell growth and survival. In the presence of anti-angiogenic compounds, tumor growth and metastasis is inhibited. Such anti-angiogenesis therapy can be used alone, or in combination with other cancer therapies to treat and/or otherwise prevent the progression of cancer.

The invention provides methods and compositions for inhibiting angiogenesis in a subject with aberrant angiogenesis. In light of the well-recognized role for anti-angiogenics in the treatment of many types of cancer, the present invention provides methods and compositions for the treatment of cancer and metastasis of tumors. For example, the present invention provides methods and compositions to inhibit the growth, survival, or metastasis of a tumor or of tumor cells, by inhibiting the natural role Norrin plays in angiogenic stimulation when it is involved in Norrin:Fz4 signaling. Given the role that Norrin plays in the angiogenesis of blood vessels in the normal retina, and ear, it is believed that cancer cells, including solid tumors, may employ Norrin to activate Fz4 and provide a selective advantage in Wnt mediated signaling.

### EXAMPLES

### Example 1

### Experimental Procedures

### A. Reagents

Dual Luciferase assay kit was purchased from Promega. Heparin was purchased from Sigma. HEK293 and COS-1 cells were purchased from the American Tissue Culture Collection (ATCC). The 293STF cells (HEK293 cells with an integrated “Super-Top-Flash” TCF-luciferase reporter) have been described previously (Xu et al., 2004). Biotinylation of MBP-Norrin protein was performed by using the EZ-Link NHS-PEG4-Biotin kit (Thermo Scientific) according to the manual's instruction. Normal human IgG was purchased from Invitrogen. A cyclic peptide (biotin-GGGGGGCNSIKFCG) with N-terminal biotinylation and a disulfide bond between two cysteines was based on the DKK1 peptide from a previous publication (Bourhis et al., 2011) and purchased from China Peptides Co., Ltd.

### B. DNA Plasmids

The TKRlu plasmid with the Renilla luciferase (Rlu) gene under the control of a thymidine kinase promoter was used as a transfection control (Promega). Human full-length Lrp5 and Lrp6 plasmids have been described (Holmen et al. 2005). The Lrp5-NT truncation construct was created by cloning Lrp5 ECD, the transmembrane (TM) domain, and eight residues following the TM domain (residues 1-1416) into the pcDNA6 vector with a v5 and a His6 tag at the C-terminus. The Lrp6 BP1 (residues 20-325) and BP2 (residues 325-631) fragments were cloned into a pcDNA6 vector that had been previously cloned with a murine Igκ leader sequence (pcDNA6-IgL) plus Fc and His6 tags to create Lrp6BP1-FcH6 and Lrp6BP2-FcH6 expression constructs. Human Norrin, Fz4, and Fz8 plasmids were purchased from Open Biosystems. Xenopus Fz8 was purchased from Addgene. The pHL-FcH6 vector from E. Yvonne Jones was cloned with a murine Igκ leader sequence to create the pHL-IgL-FcH6 vector, which allows target protein secretion into medium supernatant. The CRD regions of Fz4 (residues 40-164) or Fz8 (residues 28-155) were cloned into the pHL-IgL-FcH6 vector to create the Fz4-FcH6 and Fz8-FcH6 fusion constructs. To purify Fz4 CRD, a thrombin site was engineered between the Fz4 CRD and the Fc tag. The Fz4 CRD was also cloned in fusion with T4 lysozyme (residue 2-161, with C54T and C97A mutations) and included a v5 and His6 tandem tag at the C-terminus (Fz4-T4L-v5H6). T4 lysozyme with the same tandem tag was used as a negative control (T4L-v5H6). Both cDNAs were cloned into pcDNA6-IgL vector. The full-length human Fz4 cDNA was first cloned into the pcDNA3.1 expression vector (Invitrogen). The v5, YFP, or Rlu cDNAs were subsequently cloned at the protein C-terminus of Fz4 to create an in-frame fusion protein for v5-, Rlu- and YFP-tagged Fz4 receptor constructs. The YFP-N (residues 1-158) and YFP-C (residues 159-239) tagged Fz4 constructs were made by cloning YFP-N or YFP-C fragments at the protein C-terminus of Fz4. Human Norrin (residues 25-133) in fusion with maltose binding protein (MBP) at its N-terminus was cloned into the first cloning site of a pETDuet1 vector (Novagen), which also included a Dsbc cDNA at the second cloning site. For in vivo biotinylation of MBP-Norrin, a DNA oligonucleotide duplex encoding a biotinylation peptide (AviTag) was inserted in-frame at the 5′-end of the MBP-Norrin cDNA. In addition, the biotin ligase (BirA) gene with a T7 promoter was cloned downstream of MBP-Norrin cDNA. Coexpression of MBP-Norrin and BirA in the presence of biotin allowed in vivo biotinylation of MBP-Norrin (Smith et al. 1998). The MBP-Norrin cDNA was also cloned into pcDNA6-IgL vector. Norrin was cloned into the pHL-IgL vector for mammalian expression. All Norrin mutations were generated by PCR. Human Tspan12 (DNASU Plasmid Repository) was cloned into pCDNA3.1 expression vector (Invitrogen). The YFP or Rlu cDNAs were cloned at the C-terminus of Tspan12 to create an in-frame fusion for Rlu- and YFP-tagged Tspan12 constructs. All DNA constructs were verified by automated DNA sequencing.

### C. Protein Expression and Purification

Norrin protein was expressed as a fusion protein with MBP at its N-terminus in the E. coli strain Origami B (DE3) (Novagen) as previously described (Pioszak and Xu, 2008). The purification protocol was as previously described for the MBP-PTH1R ECD fusion protein (Pioszak and Xu, 2008), with the following modifications. Briefly, Norrin is expressed as a fusion protein with MBP from the expression vector pET-Duet1 in the strain Escherichia coli Origami (DE3) (Novagen), which contains trxB gor mutations that promote disulfide bond formation. Cells harboring this expression plasmid are grown in LB broth to midlog phase at 37° C., cooled to 16° C., and induced with 0.4 mM IPTG for ≈18 h. The cells are harvested and resuspended in buffer C [50 mM Tris.HCl (pH 7.5), 10% (vol/vol) glycerol, 150 mM NaCl, 25 mM imidazole] and then lysed by homogenization at 10,000 p.s.i. with an Invensys APV homogenizer (Albertslund). The lysate is centrifuged, and the supernatant was loaded on a 50-ml Ni²⁺-chelating Sepharose column (GE Healthcare). The column is washed with 400 ml of buffer C and eluted with buffer D, containing 50 mM Tris-HCl (pH 7.5), 10% (vol/vol) glycerol, 150 mM NaCl, and 250 mM imidazole.

The peak fractions are pooled and loaded on a 50-ml Amylose column (New England Biolabs). The column is washed with 100 ml of buffer E [50 mM Tris.HCl (pH 7.5), 5% (vol/vol) glycerol, 150 mM NaCl, and 0.5 mM EDTA] and eluted with a linear gradient of 0-10 mM maltose in buffer E. The peak fractions are pooled and subjected to disulfide shuffling in the presence of oxidized (GSSG) and reduced (GSH) glutathione (Sigma) and DsbC. The MBP-Norrin protein is refolded in a solution consisting of 1 M L-arginine, 20 mM Tris, pH 8.5, 0.5 M NaCl, 1 mM GSH, 1 mM GSSG, 1 mM EDTA. The active dimeric protein was further purified by a Heparin column (GE Healthcare) followed by a Superdex 200 gel filtration column (GE Healthcare). Protein concentrations were determined by the Bradford method (Bradford, 1976) with bovine serum albumin (BSA) as the standard. At this point, the protein is approximately >95% pure as judged by SDS/PAGE and native/PAGE. The final protein is dialyzed against a storage buffer containing 50 mM Tris.HCl (pH 7.5), 50% (vol/vol) glycerol, 150 mM NaCl, and 1 mM EDTA and is subsequently stored as aliquots at −80° C. A yield of ≈25 mg is obtained from a 6-liter culture.

The MBP tagged Norrin mutant proteins are purified similarly as the wild-type. The expression and purification of the Lrp6 extracellular β-propeller domain fragments, Lrp6 BP1-2, Lrp6 BP3-4 and Lrp6 BP1-4 proteins have been described before (Cheng et al., 2011). The Lrp6BP1-Fc(His)6 and Lrp6BP2-Fc(His)6 proteins are expressed by transient transfection of HEK293 cells with the corresponding DNA plus the MESD expression vector using lipofectamine 2000 (Invitrogen) according to the manual's instruction. The Fz4-Fc and Fz8-Fc proteins are expressed similarly by transient transfection of HEK293 cells with the corresponding DNA using lipofectamine 2000 (Invitrogen). The media supernatants are collected after four days and dialyzed against TBS buffer (20 mM Tris, pH 8.0, 0.15 M NaCl, 5% glycerol) before purifying the proteins by NiNTA chromatography. A stable cell line secreting ˜2 μg/ml Fz4-T4L into media supernatant is selected and Fz4-T4L fusion protein is purified from media supernatant by NiNTA chromatography similarly as described for the Fz4-Fc protein. To purify Fz4 CRD protein, purified Fz4 CRD-thFc(His)₆ protein is digested with thrombin (1:500) overnight at room temperature and the Fc(His)₆ tag is separated from Fz4 CRD by passing through a 5 ml NiNTA column.

### D. Crystallization of MBP-Norrin Protein

The MBP-Norrin protein in 20 mM Tris, pH 8.0, 0.1 M NaCl, 5% glycerol, 1 mM maltose, and 1 mM EDTA was concentrated to about 5 mg/ml before setting up crystallization trials using the Phoenix crystallization robot. Several conditions yielded very good-looking crystals, and diffracted to about 6 Å. The low resolution may be due to the flexible N-terminus of Norrin. A truncation construct of Norrin (residues 31-133) fused with MBP at its N-terminus was created. The encoding protein was expressed and purified with a protocol similar to that used for the wild-type protein. The function for this truncated protein was shown by binding assays to be very similar to that of the wild-type protein (data not shown). After screening many crystals at the APS synchrotron, crystal diffraction data to 2.4 Å was obtained from crystals grown in 15% PEG 3350, 0.1 M sodium acetate, pH 4.6, and 0.2 M ammonium acetate. The crystal structure of the MBP-Norrin protein was determined by molecular replacement using MBP as an initial model. Attempts to use the structure of TGF-β3, a remote homolog of Norrin, as a search model failed to yield a correct solution. The model for Norrin was therefore built de novo with phases established by the MBP structure. The Coot program was used for model building (Emsley and Cowtan 2004). The electron density for Norrin was gradually improved over many cycles of manual model building and refinements. The final model of Norrin included all the residues, and the electron density for most of residues including side chains can be clearly seen except for that of a loop region (residues 111-116). The diffraction data and refinement statistics are listed in Table 2. The MBP-Norrin structure was deposited into PDB databank with a PDB code 4MY2.

### E. Structure Determination

The crystal structure of the MBP-Norrin protein was determined using the molecular replacement method with the Phaser program from CCP4 (Bailey, 1994) with MBP as initial model. Attempts to use the structure of TGF-β3, the homolog of Norrin, as model failed to yield any correct solution. The model for Norrin was therefore built from scratch with help from phases provided by the MBP structure, because MBP makes up about 80% of the total fusion protein. The Coot program was used for model building (Emsley and Cowtan, 2004). The electron density map for Norrin was initially noisy, but gradually improved with many cycles of manual model building and refinements. The side chains for Norrin residues were assigned with the improved density map. The final model of Norrin included all the residues and the electron density for most of residues including side chains can be clearly seen except that a loop region (residue 111-116) cannot be well resolved. The diffraction data and refinement statistics were listed in Table 2.

TABLE 2
X-ray diffraction data and refinement statistics.

| Data collection | MBP-Norrin |
|---|---|
| APS beamline | 21-ID-G |
| Space group | P2₁2₁2 |
| Resolution, Å | 50-2.4 |
| Cell parameters, Å; ° | a = 59.5, b = 79.0, |
| | c = 104.2; α = β = γ = 90° |
| Total/unique reflections | 188,712/19,865 |
| Completeness, % | 100.0 (100.0) |
| I/σ | 13.1 (3.0) |
| Redundancy | 9.5 (9.0) |
| R_{sym} | 0.156 (0.883) |
| Structure determination | |
| Resolution, Å | 50-2.40 |
| No. reflections | 18,833 |
| No. residues | 474 |
| No. solvent molecules | 202 |
| No. of non-H atoms | 3913 |
| R_{cryst} | 19.3% |
| R_{free} | 23.9% |
| rmsd bonds, Å | 0.013 |
| rmsd angles, ° | 1.76 |
| Average B factor, Å² | 34.6 |
| Ramachandran statistics | |
| Most favoured regions (%) | 90.8 |
| Additional allowed regions (%) | 8.9 |
| Generously allowed regions (%) | 0.2 |
| Disallowed regions (%) | 0 |

### F. Alpha Screen

AlphaScreen assay is a bead-based luminescence proximity assay. The binding between biotinylated MBP-Norrin and His6-tagged Fz4-Fc, Fz8-Fc, or His8 tagged Lrp6 ECD proteins was determined by the AlphaScreen assay using a hexahistidine detection kit from Perkin-Elmer. In this assay, biotinylated MBP-Norrin was attached to streptavidin coated donor beads and His6 tagged protein was attached to Ni-chelate coated acceptor beads. When the donor and acceptor beads were brought into proximity by the interaction between MBP-Norrin and His6 tagged protein, illuminating the sample with a laser at 680 nm released singlet oxygen molecules from donor beads to acceptor beads, which then elicited a strong emission of light at a shorter wavelength (520˜620 nm). The binding mixtures, containing the indicated amounts of proteins, 10-20 μg/ml of streptavidin-coated “donor” beads, and Ni-chelate-coated “acceptor” beads, were incubated in 50 mM MOPS pH 7.4, 100 mM NaCl, and 0.1 mg/ml BSA for 2-5 h, followed by data collection using an Envision plate reader (PerkinElmer). For competition assays, increasing concentrations of unlabeled protein were added to the two labeled proteins.

### G. Biolayer Interference Assays

Binding curves were measured by biolayer interferometry using an Octet Red instrument (ForteBio). For the biolayer interferometry assay, a layer of molecules attached to the tip of the biosensor creates an interference pattern at the detector. Any change in the number of molecules bound due to protein-protein interactions causes a measured shift in the interferometric profile. When this shift is measured over a period of time and its magnitude plotted as a function of time, a classic association/dissociation curve is obtained. The protein A sensor (immobilized with protein A, a bacterial surface protein that binds the Fc region of the heavy chain of most antibodies.) or anti-human IgG Fc capture sensor (immobilized with anti-human Fc-specific antibody) was used to bind Fz4-FcH6, Fz8-FcH6, Lrp6 BP2-FcH6, or human IgG proteins in kinetics buffer (PBS, pH 7.4, 0.01% BSA, and 0.002% Tween 20) or as noted otherwise. The protein-loaded biosensors were incubated sequentially with kinetic buffer in the baseline step, the purified MBP-Norrin in solution in the association step and kinetic buffer in the dissociation step to detect their binding to MBP-Norrin protein.

### H. Cell-Based Luciferase Assay

To measure the TCF reporter activity, 293STF cells with an integrated “Super-Top-Flash” TCF-luciferase reporter were maintained in DMEM (GIBCO) with 5% fetal bovine serum (FBS). Cells were plated in a 24-well plate 24 h prior to transfection. Cells were transfected with the indicated plasmids plus 5-10 ng of the control plasmid TKRlu (constitutive expression of Renilla luciferase) using Lipofectamine 2000 (Invitrogen). The medium was changed to the low-serum opti-MEM (Invitrogen) on day 2. For experiments involving Norrin DNA transfections, cells were incubated for another 24 h before harvesting. For experiments involving protein treatments, MBP-Norrin protein was added at the time of medium change, and cells were incubated with the indicated protein for 24 h before harvesting. The cells were lysed with 100-150 μl passive lysis buffer (Promega) at room temperature for 15 min, and the firefly and Renilla luciferase activities were measured on an Envision luminometer (PerkinElmer) with the Dual Luciferase assay kit (Promega). Firefly luciferase raw data were normalized to Renilla luciferase raw data.

### I. Immunoprecipitation

To produce secreted Fz4-T4L-v5H6 or T4L-v5H6 proteins, HEK293 cells in 10-cm plate were transfected with 12 μg of the Fz4-T4L-v5H6 or T4L-v5H6 DNAs by lipofectamine 2000 (Invitrogen), and medium supernatants were collected after 4 d of transfection. To produce Lrp5NT-v5H6 protein, HEK293 cells in 10-cm plate were transfected with 12 μg Lrp5NT-v5H6 DNA for 2 d. After that, cells were harvested with Cell Dissociation Solution (Sigma) and lysed in 300 μl of cell lysis buffer (Cell Signaling) on ice for 30 min. Cell debris was removed by centrifugation. For immunoprecipitation, 30 μl of v5-agarose beads (Sigma) was used to pull down about 2 μg of Fz4-T4L-v5H6 or T4L-v5H6 from medium supernatant or Lrp5NT-v5H6 protein from 100 μl of cell lysate. The v5 beads were then incubated with or without 10 μg of MBP-Norrin and 10 μg of Fz4FcH6 proteins for 1 h at 4° C. with mixing. The v5 beads were washed with 1×TBST buffer two times and then were incubated with 100 μl of 2×SDS loading buffer to release the bound proteins. The samples were analyzed by Western blot as described previously (Ke et al. 2009; Ke et al. 2012). The blot was probed first with anti-hum IgG HRP (Santa Cruz) for Fc4-FcH6 protein. Then the blot was stripped and reprobed with a goat anti-Norrin antibody (R&D systems). The blot was stripped again and reprobed with a mouse anti-v5 antibody (Thermo Scientific).

### J. BRET Assays for Receptor Oligomerization

For BRET assays, DNA constructs in eukaryotic expression vectors were expressed transiently in COS-1 cells. COS-1 cells were seeded at a density of 0.5×10⁶ cells/dish in 10-cm tissue culture dishes in DMEM supplemented with 5% Fetal Clone II. When the cells reached 80% confluence, they were transfected with 3 μg of DNA/dish using the diethylaminoethyl (DEAE)-dextran method (Harikumar et al. 2007). Assays were performed 48-72 h later. BRET studies were performed using cells in suspension and a 2103 Envision plate reader (PerkinElmer, Waltham, Mass.) configured with a <700 nm dichroic mirror and with dual emission filter sets for luminescence (460 nm, bandwidth 25 nm) and fluorescence (535 nm, bandwidth 25 nm). These studies used approximately 25,000 cells/well in 96-well Optiplates. The studies were initiated by adding 5 μM coelenterazine h, a specific substrate for Rlu, after which the luminescence and fluorescence signals were promptly collected. Total YFP fluorescence emission was also acquired to determine acceptor concentration by exciting the samples at 485 nm and detecting the emission at 525 nm. The net BRET ratios were calculated based on the ratio of emission signals from YFP and Rlu, and corrected BRET ratios were calculated as described previously (Harikumar et al. 2007). Saturation BRET studies were performed to evaluate the specificity of the BRET signals as described previously (Harikumar et al. 2007).

### K. Fluorescence Microscopy & Fluorescence Spectroscopy

Fluorescence microscopy was used to demonstrate YFP fluorescence at the surface of the transfected cells. COS-1 cells were transfected with either the intact YFP construct or the complementary YFP-N and YFP-C constructs, as described previously (Harikumar et al. 2008a; Harikumar et al. 2008b). Cell surface YFP fluorescence was evaluated using a Zeiss Axiovert 200M epifluorescence inverted microscope with a dedicated YFP filter set (excitation 480 nm, dichroic mirror Q515 lp, emission 525 nm). Images were collected using a monochromatic ORCA-12ER CCD camera (Hamamatsu, Bridgewater, N.J.) with QED-InVivo 2.039 acquisition software (Media Cybernetics, Silver Spring, Md.). Steady-state fluorescence intensity measurements were performed in a Fluoromax-3 fluorometer (SPEX industries, Edison, N.J.) with samples at room temperature in a 1 ml quartz cuvette. Transfected cells were harvested and transferred into a 1 ml cuvette where the sample was excited using 480 nm light, and YFP fluorescence emission was monitored between 500 nm to 600 nm. Background fluorescence was corrected by analogous measurements using untransfected cells. Fluorescence anisotropy measurements were performed as described previously (Harikumar et al. 2008a).

### L. Triton X-114 Phase Separation Assay

The phase separation assay was performed as previously described (Willert et al. 2003). HEK293 cells were transiently transfected with MBP-Norrin expression vector and the medium supernatant was collected after 4 d and concentrated. As a control, HEK293 cells were transiently transfected with MBP-Rhodopsin expression vector for 1 d and the cells were lysed using a solution of 0.5% n-dodecyl-β-D-maltoside, 0.1% cholesterol, 150 mM NaCl, 20 mM Tris, pH 7.5 at 4° C. The cell lysate was centrifuged at 40,000 rpm for 30 min and the supernatant was used for the phase separation assay. MBP-Norrin conditioned media or MBP-Rhodopsin lysate were mixed 1:1 with ice cold 4.5% Triton X-114, 150 mM NaCl, 10 mM Tris-HCl, pH 7.5, and incubated on ice for 5 min, then at 31° C. for 5 min, and centrifuged at 2,000 g at 31° C. for 5 min. The top, aqueous phase was separated from the bottom Triton X-114 detergent phase and equal volumes were analyzed for Western blot.

### Example 2

### Results

### A. Active Norrin Dimer can be Purified as a MBP Fusion Protein

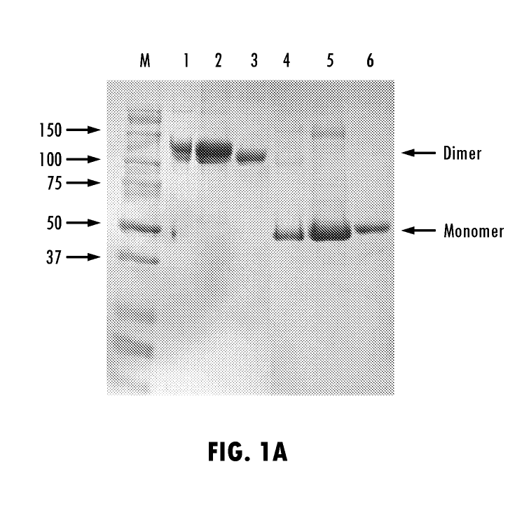

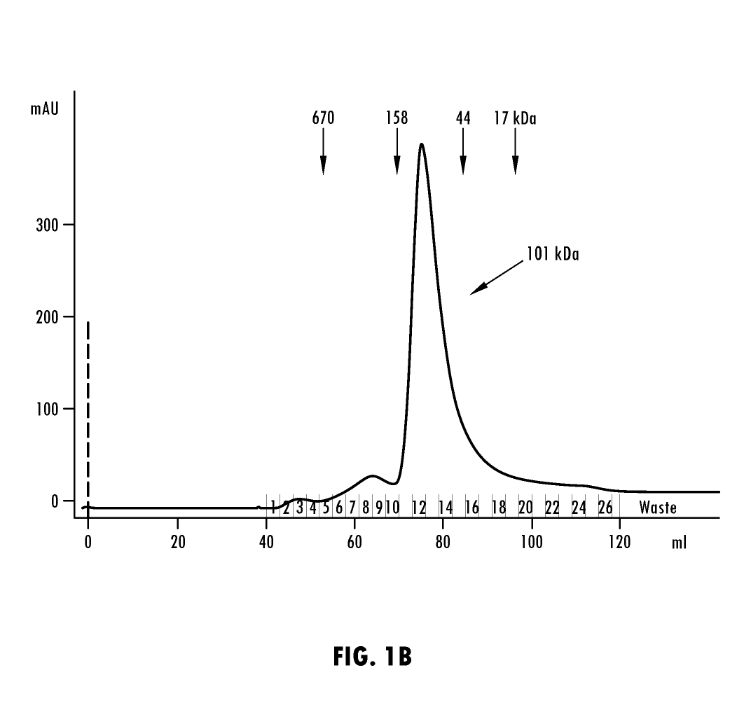

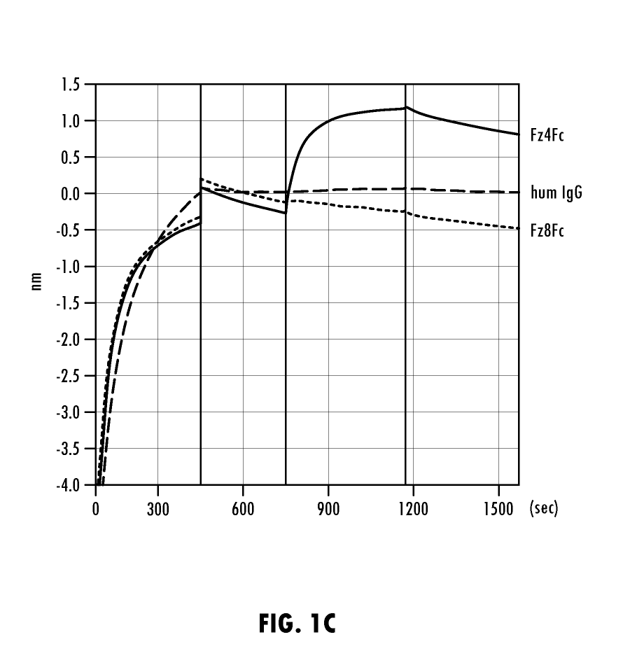

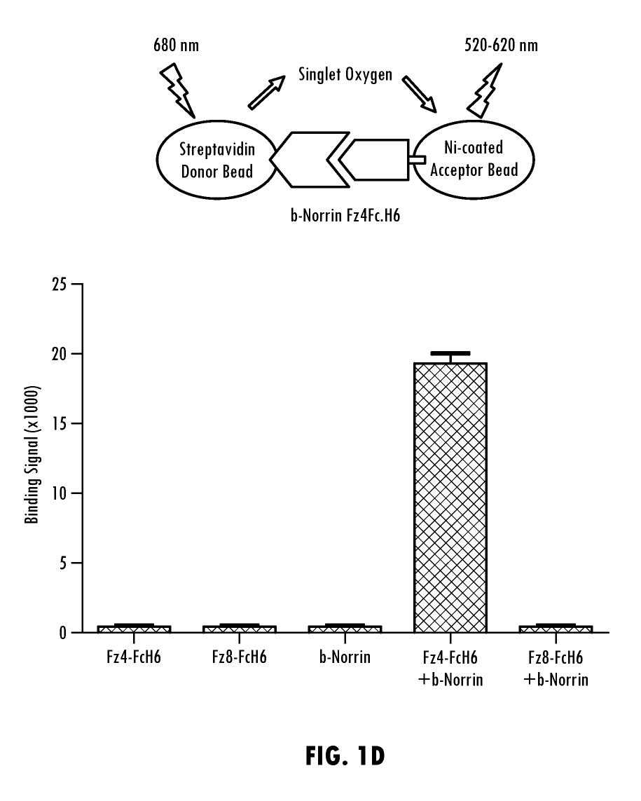

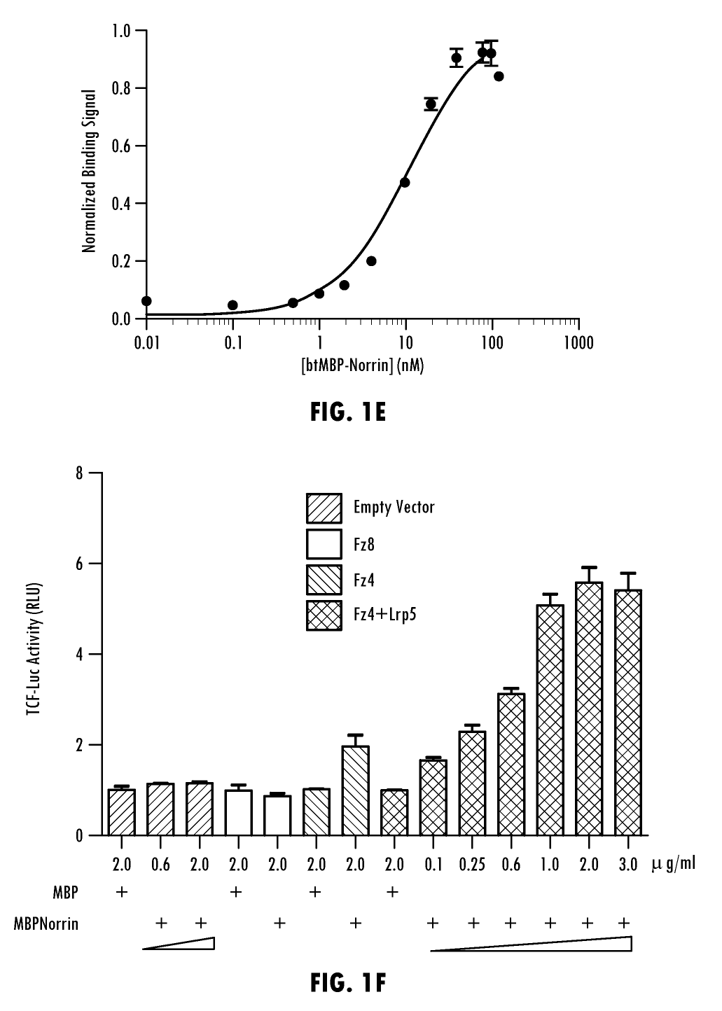

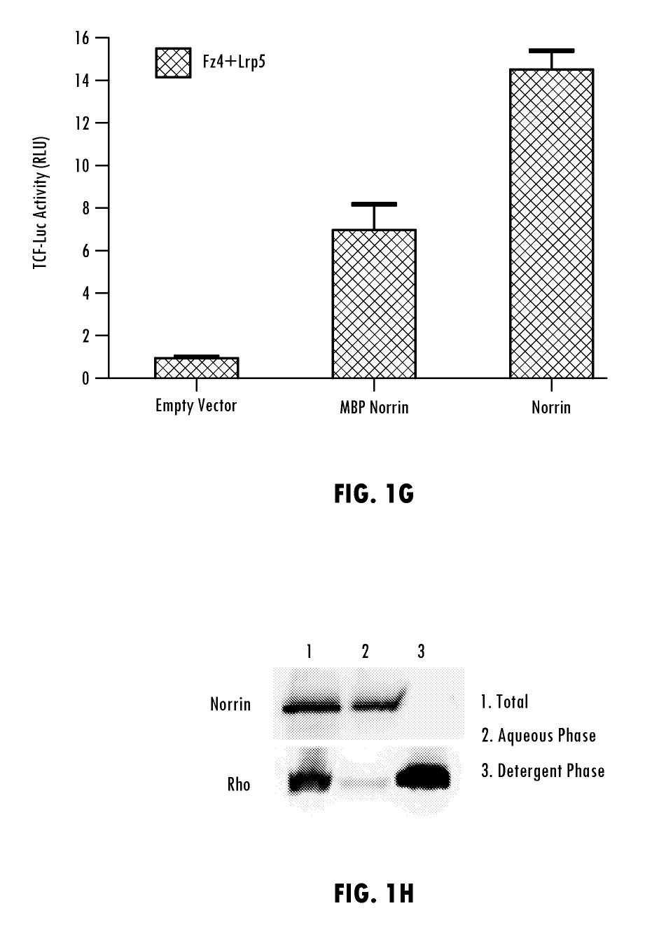

Norrin is a small protein containing 11 cysteines, and is difficult to purify from mammalian sources (Perez-Vilar and Hill 1997). Previous work that focused on purifying recombinant Norrin protein from insect cells failed to produce Norrin at high purity or in high yield (Shastry and Trese 2003). Similarly, our attempts to express Norrin in mammalian cells were not successful, while expression in standard E. coli systems resulted in the formation of inclusion bodies. We therefore developed a new expression and purification method for Norrin based on an E. coli system that we used for class B G protein-coupled receptor (GPCR) extracellular domains (ECDs), which contain several disulfide bonds (Pioszak et al. 2008; Pioszak and Xu 2008) (see Materials and methods). Using this system, human Norrin (residues 25-133, without its signal peptide) fused to the C-terminus of maltose-binding protein (MBP) could be expressed as a soluble protein (FIG. 1A).

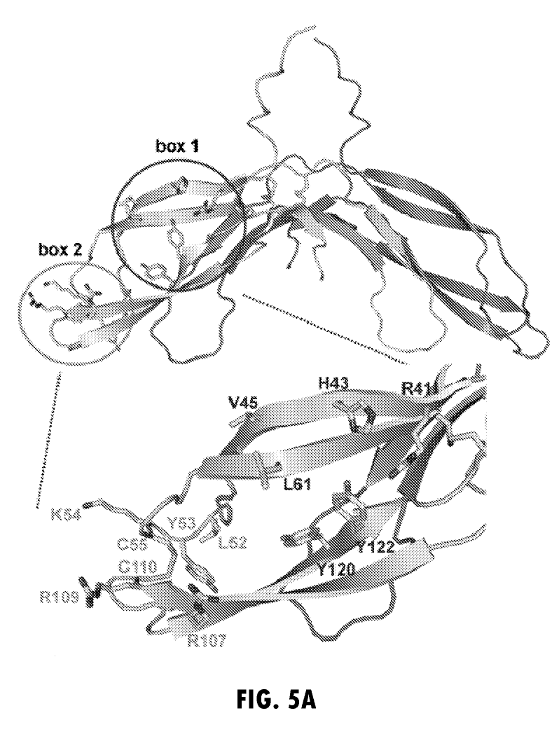

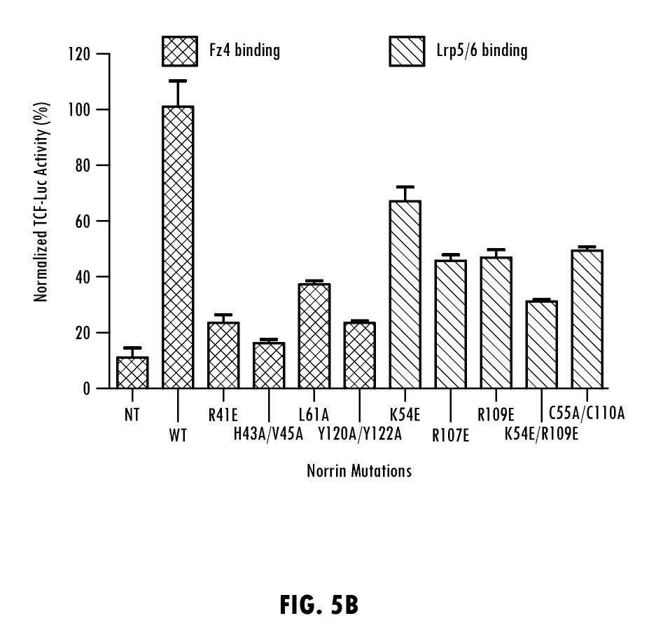

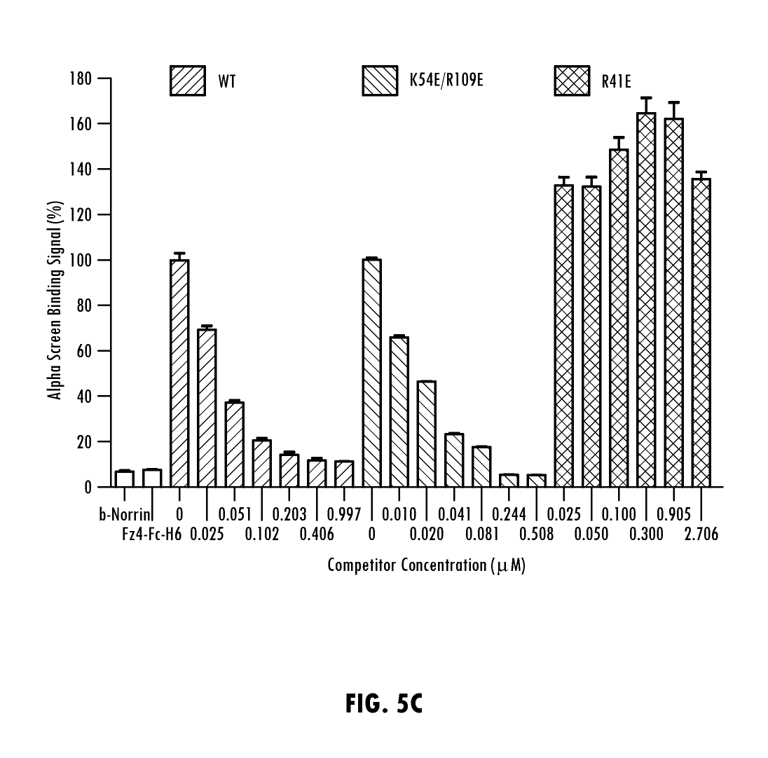

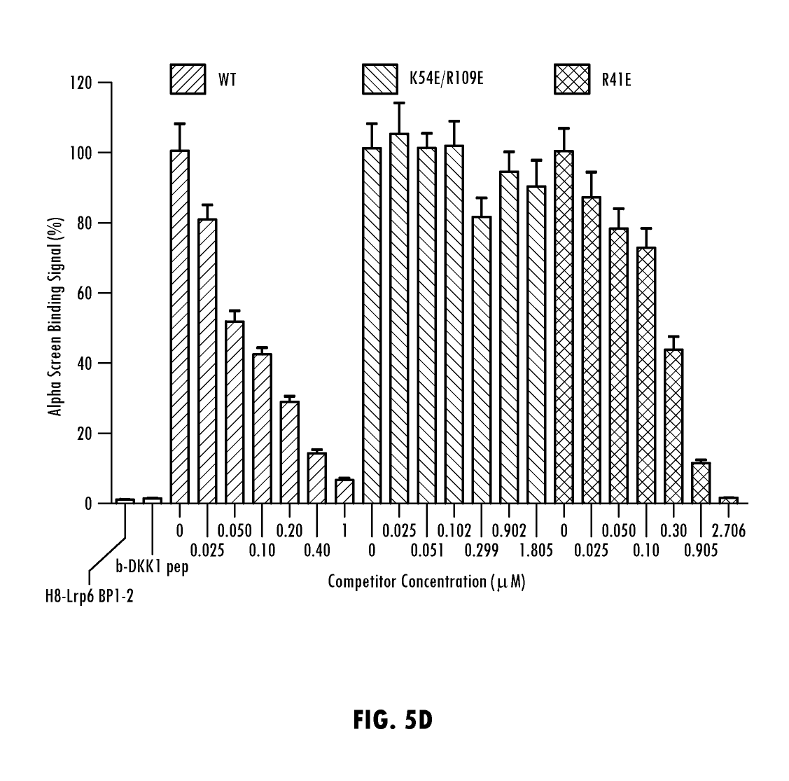

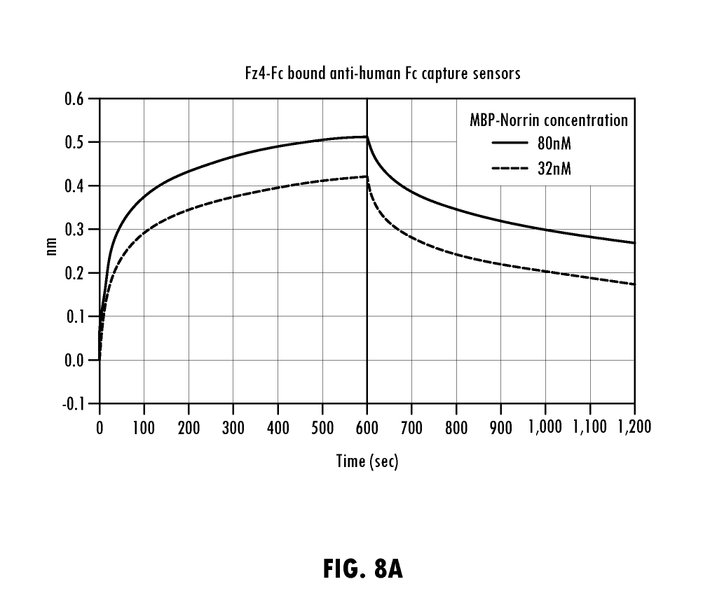

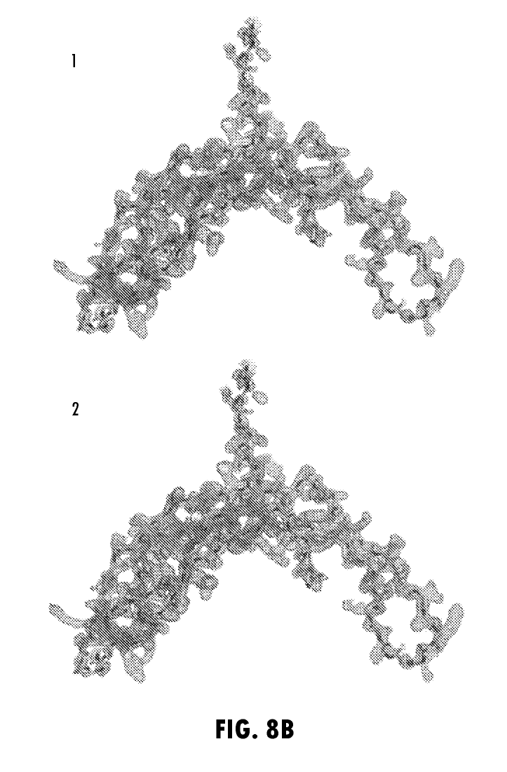

The purified MBP-Norrin protein formed disulfide bond-linked dimers that migrated at the predicted molecular weight of 106 kDa under nonreducing conditions and of 53 kDa under reducing conditions (FIG. 1A). MBP-Norrin also runs as a dimer peak on a gel-filtration column (FIG. 1B). The fusion protein was active in binding to mammalian-produced Fz4 CRD fused with an FcH6 tag (Fz4-FcH6) as determined by both biolayer interferometry (FIG. 1C) and AlphaScreen luminescence proximity assays (FIG. 1D). The binding avidity between MBP-Norrin dimer and Fz4-FcH6 dimer was determined to be approximately 11 nM by AlphaScreen saturation binding curves (FIG. 1E) and 5 nM by biolayer interferometry (FIG. 8A), which is similar to that of mammalian Norrin to Fz4 in an earlier report (Xu et al. 2004). As expected, binding was specific to Fz4 because we were unable to detect any interaction with the Fz8 CRD (FIGS. 1C, 1D). Recombinant MBP-Norrin was also active in cell-based reporter assays. 293STF cells without Fz4 expression did not respond to exogenously added MBP-Norrin (FIG. 1F). Recombinant MBP-Norrin protein increased the TCF reporter activity of cells transfected with full-length Fz4 by about twofold and of cells cotransfected with Fz4 and Lrp5 to about sixfold in a dose-dependent manner, with an EC50 of about 6 nM (0.6 μg/ml; FIG. 1F). Cotransfection of 293STF cells with Fz4, Lrp5, and MBP-Norrin mammalian expression vectors caused similar levels of activation, which are about half of those obtained by cotransfection with the untagged Norrin (FIGS. 1F, 1G). This indicates that the purified MBP-Norrin protein is active and selective for activation of Fz4, consistent with the previous report (Xu et al. 2004). To examine whether Norrin is lipid modified and therefore hydrophobic, as reported for Wnt ligands (Willert et al. 2003; Takada et al. 2006; Janda et al. 2012), a phase separation experiment was performed. MBP-Norrin expressed from mammalian cells partitioned into the aqueous phase whereas MBP-rhodopsin (a GPCR membrane protein) partitioned into the detergent phase as expected (FIG. 1H). So in contrast to Wnts, Norrin is a hydrophilic protein without detectable lipid modification, which is consistent with the fact that Norrin lacks the conserved saposin-like domain of Wnts, where the lipid modified serine residue is located.

### B. The Crystal Structure of MBP-Norrin Reveals a Novel Dimeric Complex

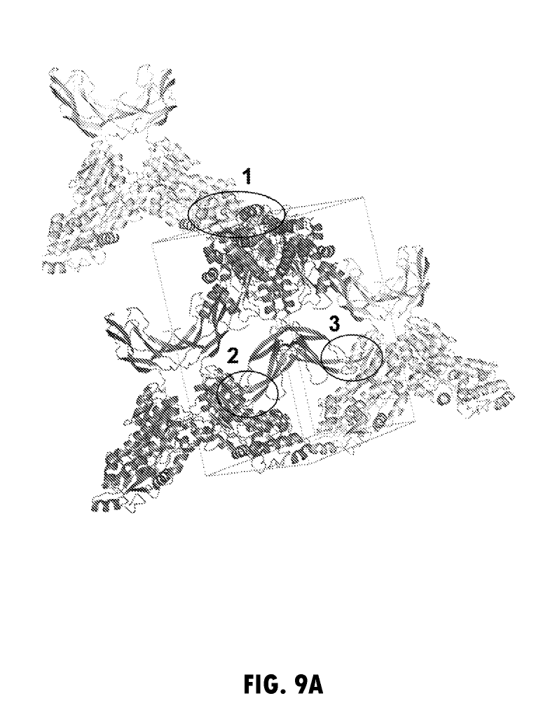

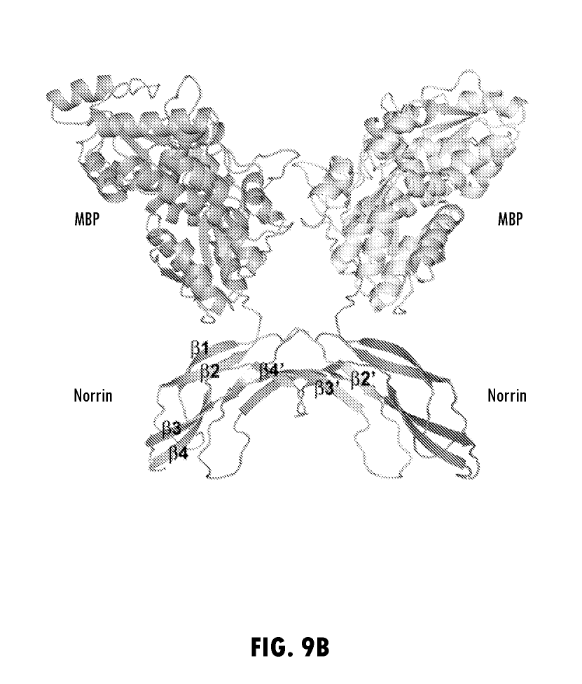

The inventors were able to generate high-quality crystals of MBP-Norrin that allowed the structure to be determined to 2.4 Å resolution (Table 2). There is excellent electron density for the Norrin molecule in the crystal (FIG. 8B). The structure was solved with the phase information derived from the MBP molecular replacement solution, which revealed that MBP is involved in crystal packing interactions (FIG. 9A). Thus, the inclusion of MBP as a fusion tag not only helped increase the solubility of Norrin, but also aided in crystallization and structure determination of the fused protein.

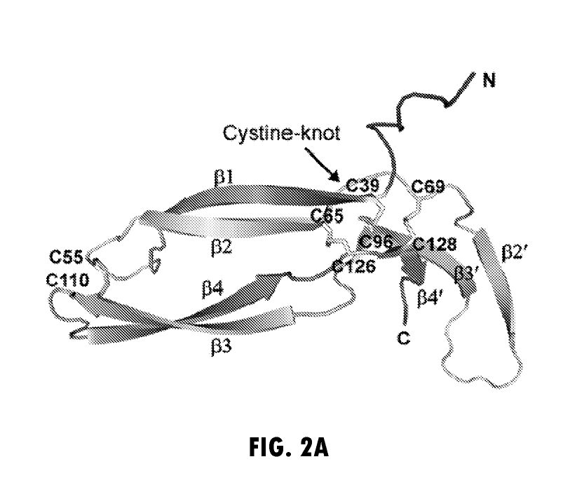

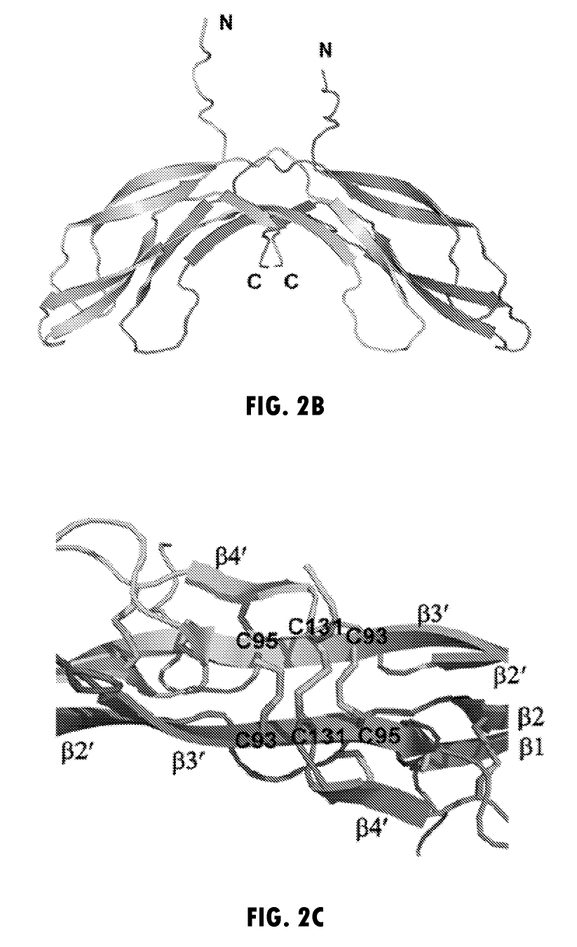

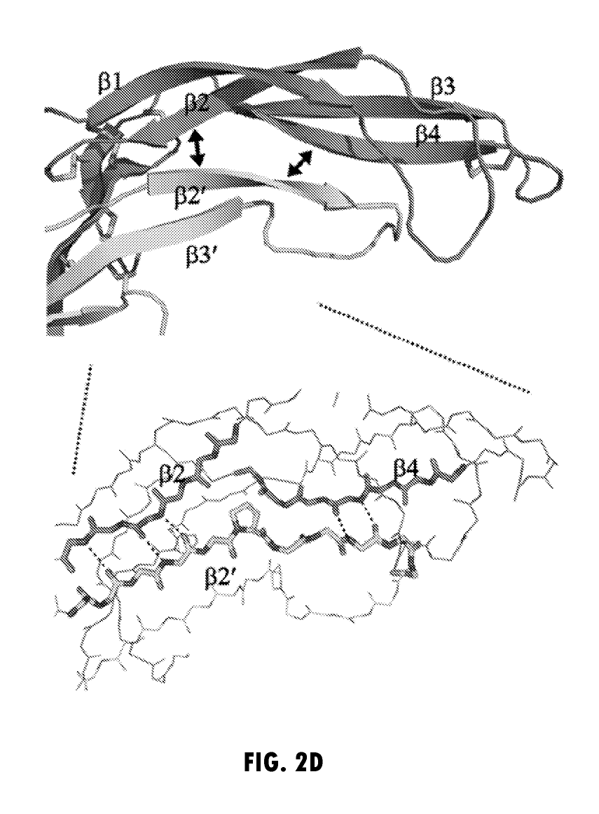

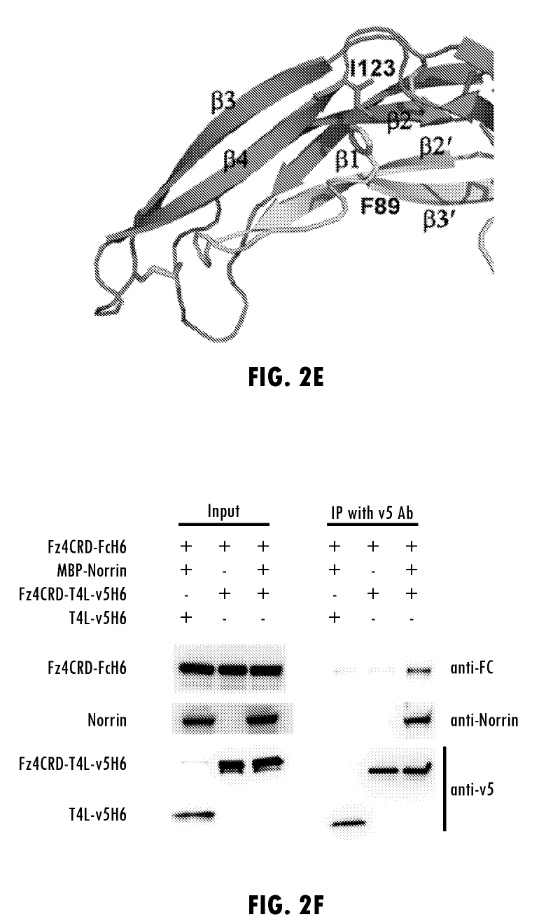

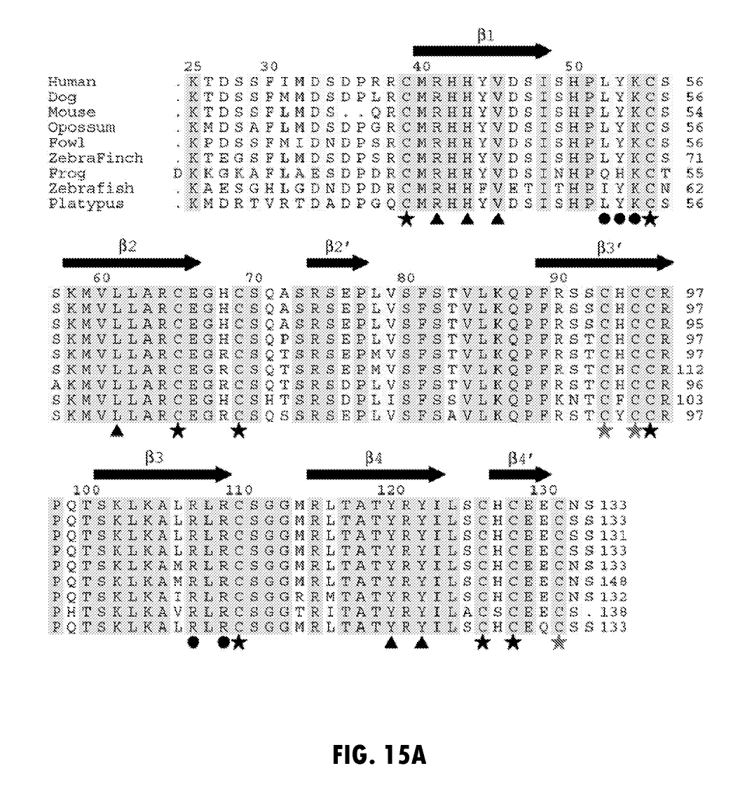

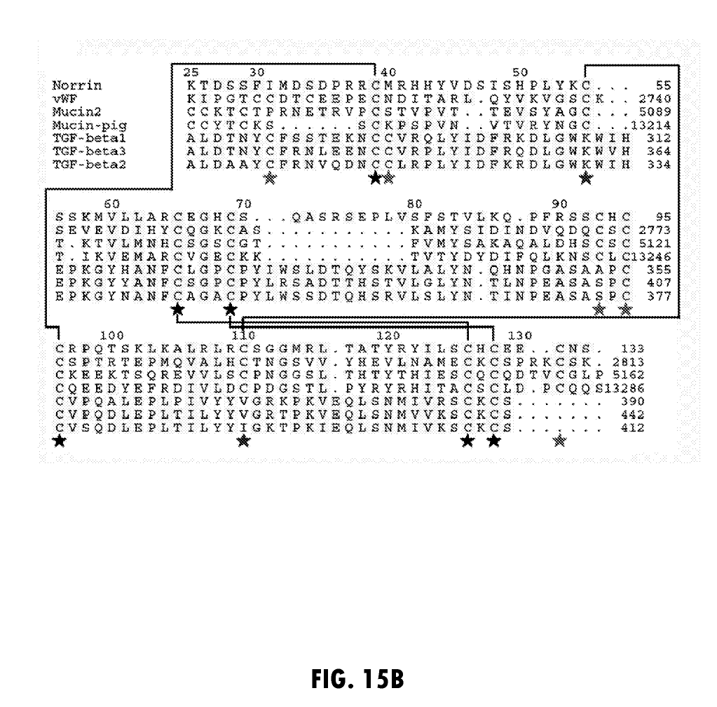

In the structure, MBP-Norrin is arranged as a homodimer with one monomer per asymmetric unit (FIG. 9B). Each Norrin monomer consists of solely β-strands and loops (no α-helices) and forms a very flat structure (59 Å×16 Å×6 Å) that includes a cystine knot motif (FIG. 2A). The motif contains an unusual clustering of three disulfide bonds, two of which, with their connecting peptide backbone, form a ring structure that is threaded by a third disulfide bond. In the Norrin structure, the cystine knot is near the center of each monomer, with two disulfide bonds (C65-C126 and C69-C128) and four amino acid backbones forming an eight-residue ring through which a third disulfide bond (C39-C96) passes (FIG. 2A, FIG. 15B). On the left side of the cystine knot are four β-strands (β1 to β4) that form two anti-parallel β-hairpins. The β-hairpins are stabilized on one end by the cystine knot structure and on the other side by a Norrin-specific disulfide bond (C55-C110) that connects the β1-β2 loop with the β3-β4 loop. On the right side of the cystine knot is an additional β-hairpin formed by strands β2′ and β3′.

The Norrin dimer is formed by two highly intertwined monomers that bury 1543 Å2 of surface area (FIG. 2B). In contrast to only one intermolecular disulfide bond (C95-C95) predicted previously (Smallwood et al. 2007), three disulfide bonds were observed between the two monomers, two disulfide bonds (C93-C95 and C95-C93) that link the two adjacent β3′ strands from each monomer, and one disulfide bond (C131-C131) that links the C-termini of both monomers (FIG. 2C). The dimeric interface is further stabilized by intermolecular hydrogen bonds from β2′ of one monomer to β2 and β4 of the other monomer (FIG. 2D). As is typical for cystine knot growth factors, Norrin monomers lack a hydrophobic core on their own, but in the context of dimers they form a well-defined hydrophobic core that is partly mediated by symmetric packing of F89 and I123 from each monomer (FIG. 2E). Its extensive dimer interface suggests that Norrin functions as a dimeric protein.

### C. Structural Comparison of Norrin to TGF-β Growth Factors

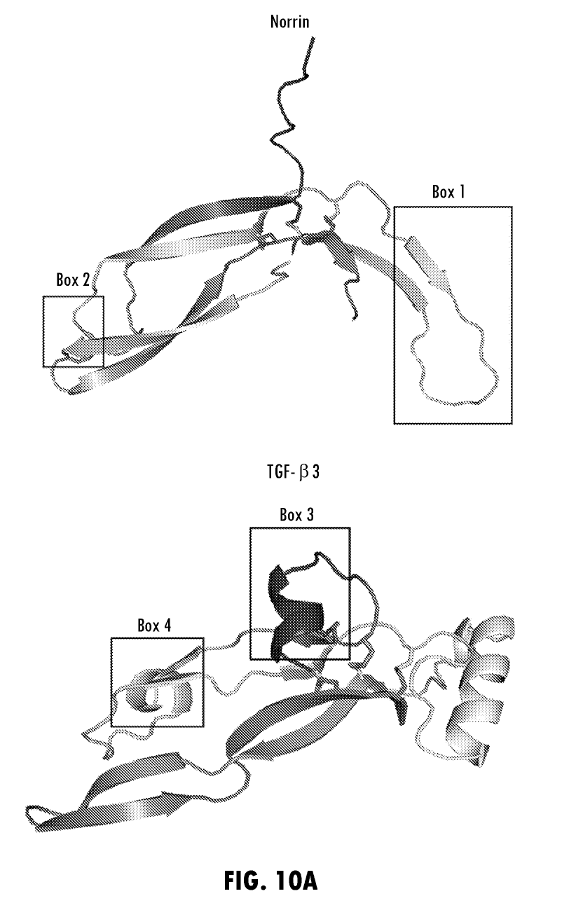

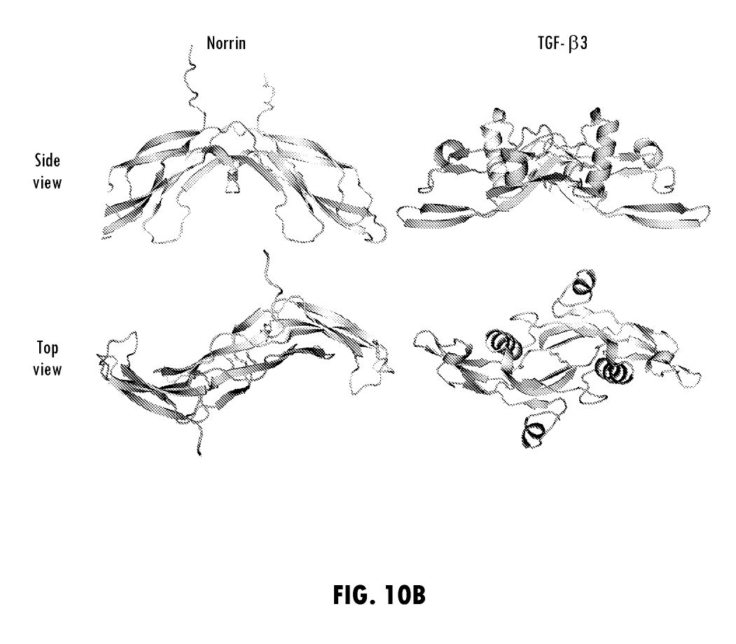

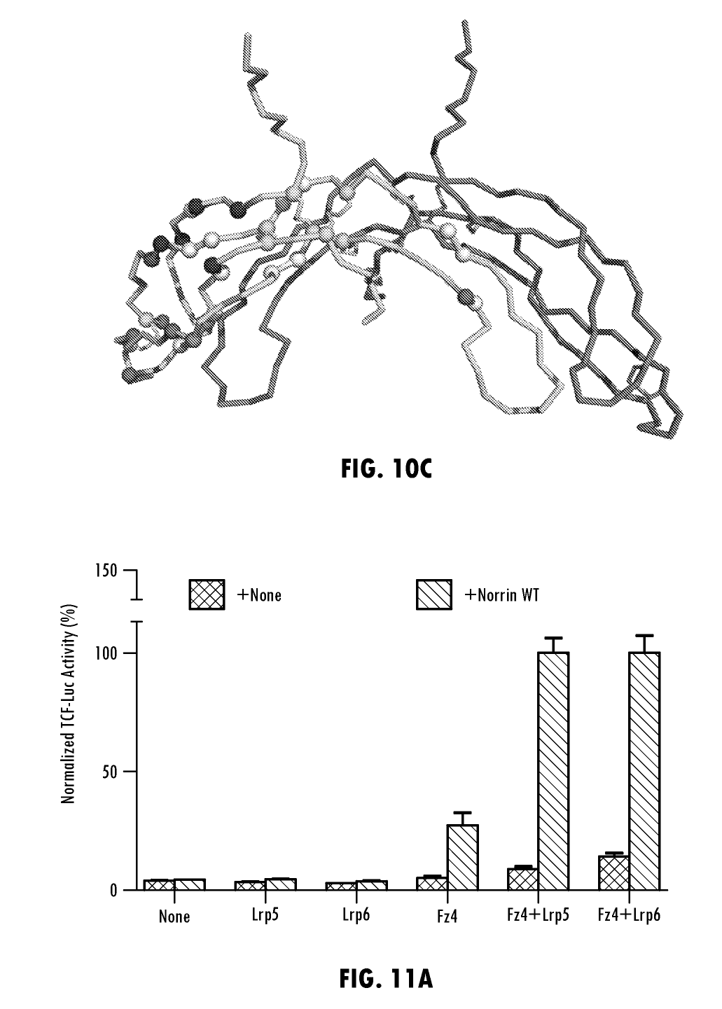

The cystine knot structural motif is found in a number of growth factors including nerve growth factor (NGF), transforming growth factor-β (TGF-β), bone morphogenetic protein, platelet-derived growth factor (PDGF-BB), and glycoprotein hormones such as human chorionic gonadotropin (Sun and Davies 1995). Using TGF-β as a typical example, the structure of Norrin was compared to that of TGF-β3 (FIG. 10A-10C). Both monomeric Norrin and monomeric TGF-β3 have a highly conserved cystine knot structure, but also have significant differences in four regions (FIG. 10A). The most noticeable difference is the insertion between β2 and β3, which forms a two-stranded β-hairpin with a long loop in Norrin (box 1) and an α-helical structure in TGF-β3. A second difference is that the β1-β2 and β3-β4 loops are linked by a disulfide bond in Norrin (box 2) but are not constrained in TGF-β3. Other differences are the N-terminus and the region between β1 and β2, which form α-helices in TGF-β3 (boxes 3&4), but form loop structures in Norrin. Norrin has an overall L-shaped structure, whereas that of TGF-β3 is more planar. Both Norrin and TGF-β proteins form homodimers. TGF-β3 forms a single disulfide bond-linked dimer whereas Norrin forms a dimer linked by three disulfide bonds, with the Norrin dimer having a more extended, curved shape due to the unique insertion between β2 and β3 (FIG. 10B). The comparison suggests that the cystine knot structural fold is very plastic and can tolerate many different variations, while dimerization is a common feature to maintain the structural integrity.

### D. The Dimeric Interface of Norrin is Important for its Function

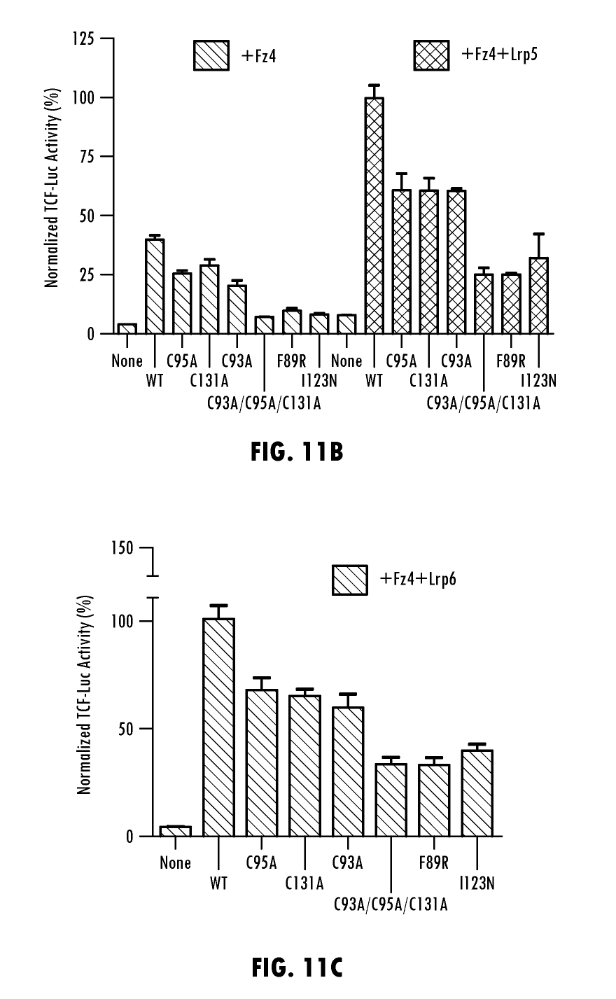

To understand the role of the dimer conformation for Norrin function, the three intermolecular disulfide bond-forming cysteines were mutated either individually (C93A, C95A, or C131A) or in combination (C93A/C95A/C131A). The mutant and wild-type Norrin constructs were transfected with Fz4 or Fz4 plus Lrp5/6 to assess their ability to activate the β-catenin/TCF luciferase reporter. Transfection of wild-type Norrin with Fz4, but not with Lrp5 or Lrp6, activated the TCF reporter, and cotransfection with Fz4 plus Lrp5/Lrp6 further increased reporter activity (FIG. 11A). Previous work showed that the C95A mutant of Norrin still forms disulfide bond-linked dimers (Perez-Vilar and Hill 1997). Consistently, single mutation of one of the three intermolecular disulfide bond-forming cysteines resulted in modest reduction of TCF reporter activity in the presence or absence of Lrp5/6 (FIGS. 11B, 11C). In contrast, mutation of all three cysteines nearly abolished the reporter activity in the absence of Lrp5/6 and reduced over half of the reporter activity in the presence of Lrp5/6 (FIGS. 11B, 11C). Together, these data support the importance of the intermolecular disulfide bonds for dimer formation and for Norrin activation of Fz4. F89 and I123, were also mutated, the two key residues that form hydrophobic interactions at the dimeric interface (FIG. 2E), to the hydrophilic residues arginine and asparagine, respectively. The F89R mutant reduced the TCF reporter activity to nearly the same degree as the triple cysteine mutant; the effect of the I123N mutation (a Norrie disease mutation) was slightly less severe (FIGS. 11B, 11C). These data further support the important role of the hydrophobic interface for the integrity of Norrin structure and function.

### E. Fz4 Receptors Form Stable Dimers in the Absence of Norrin

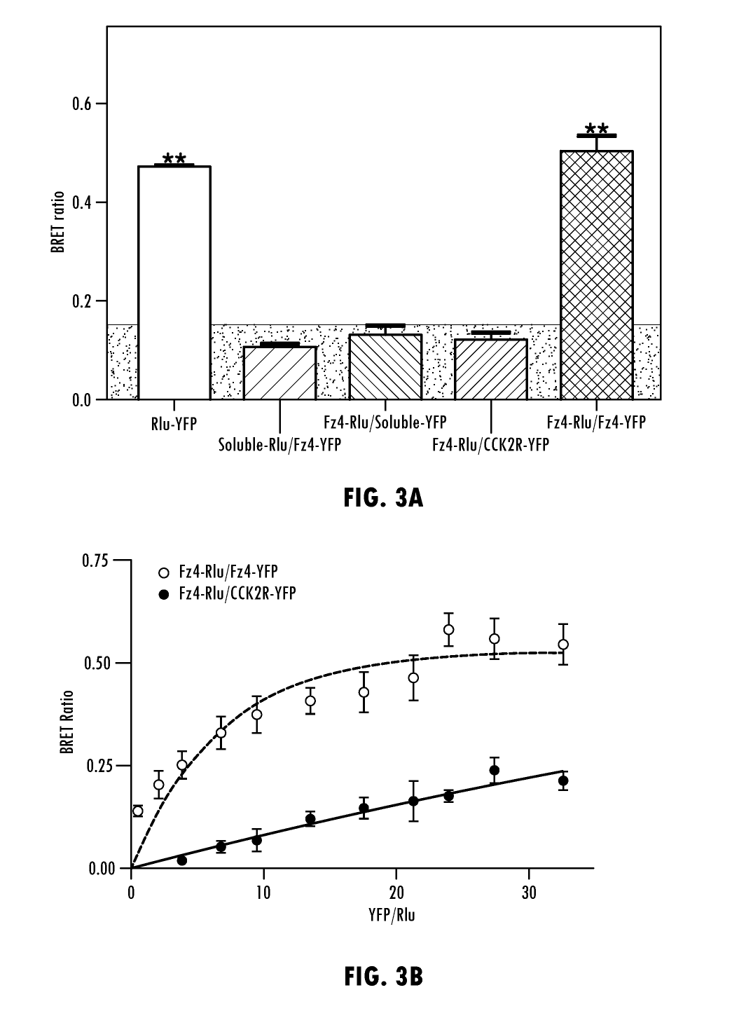

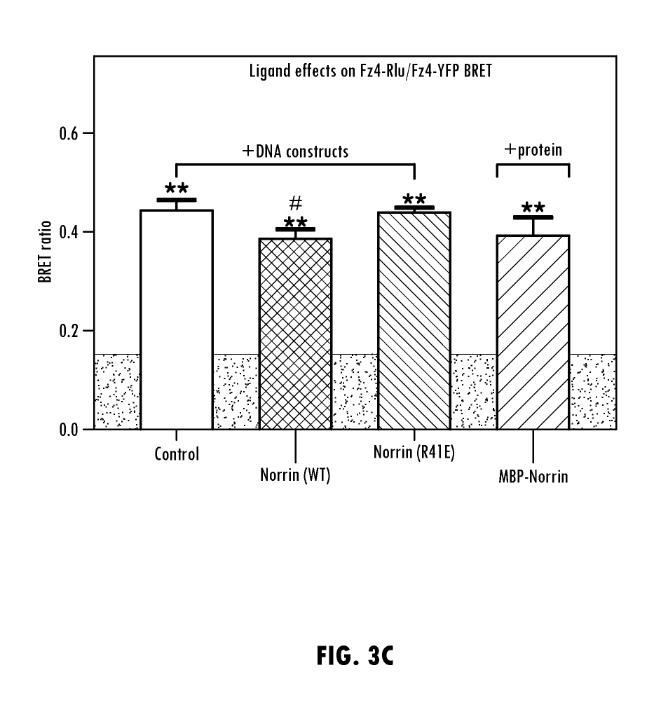

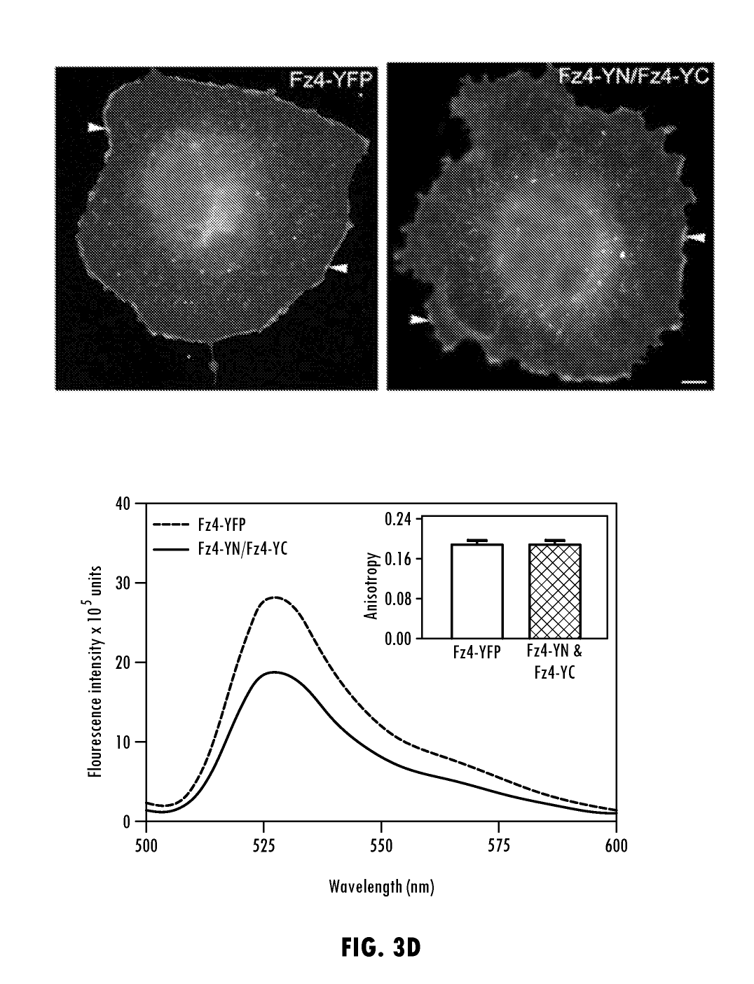

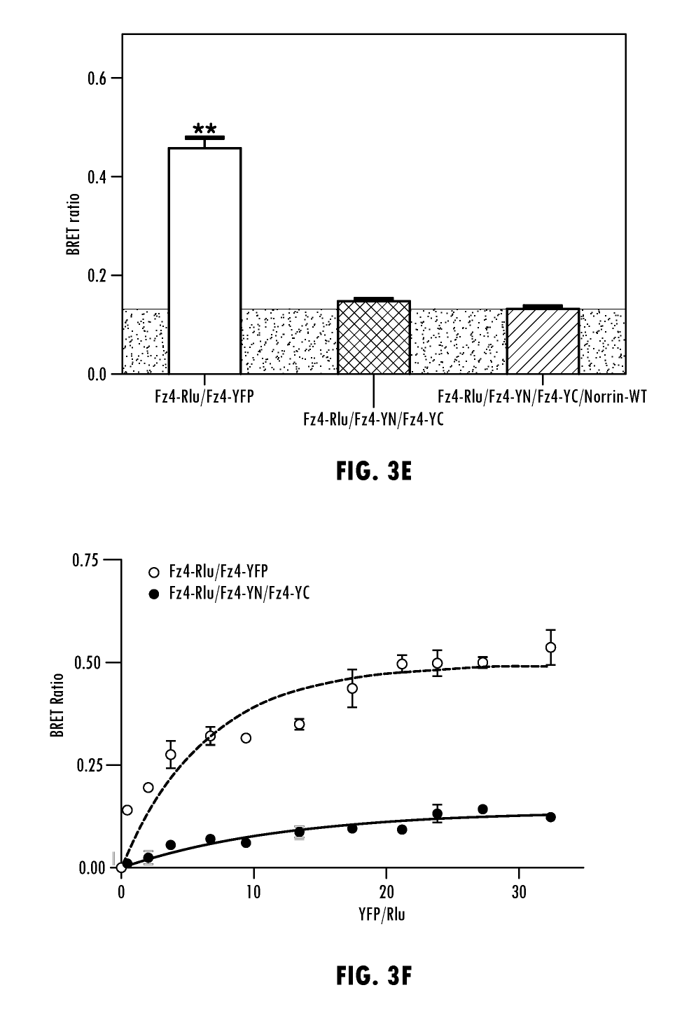

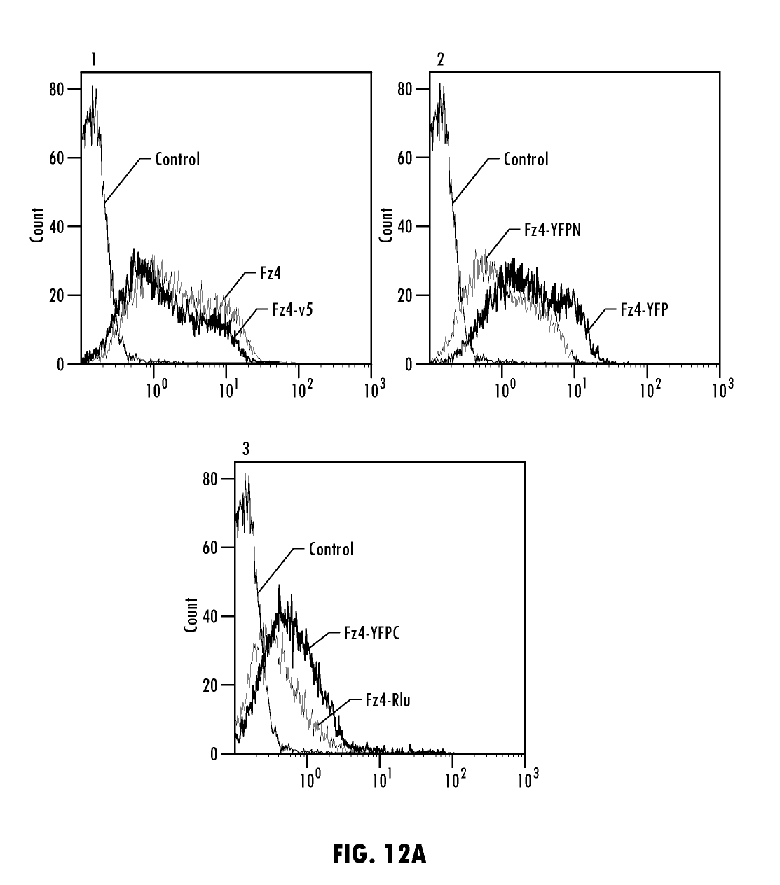

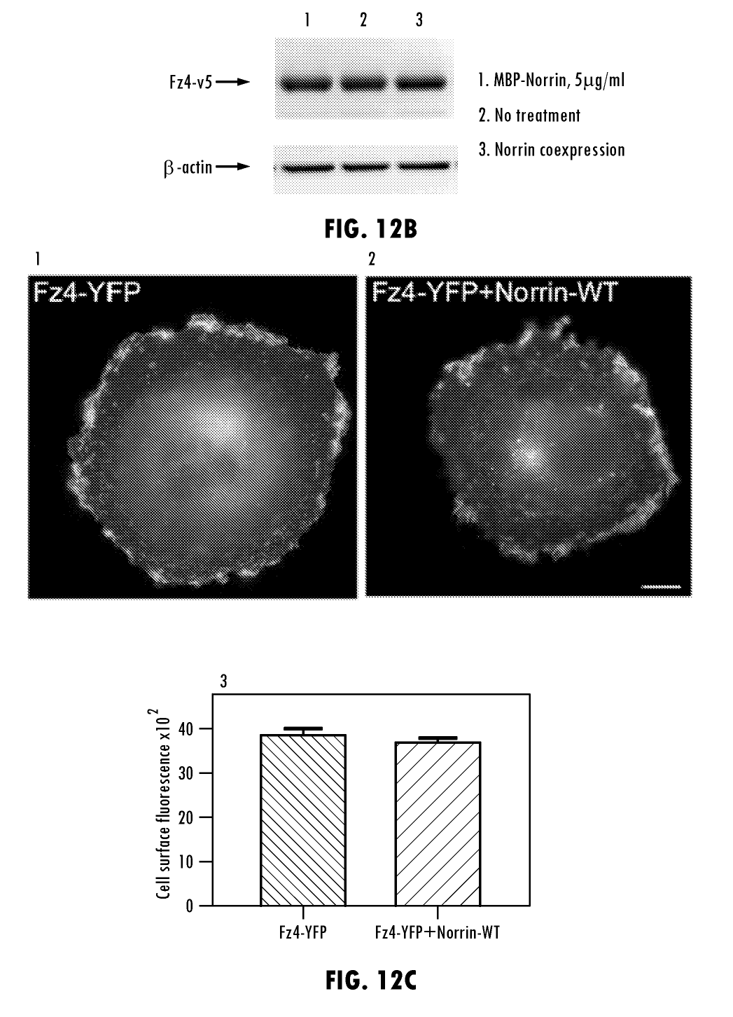

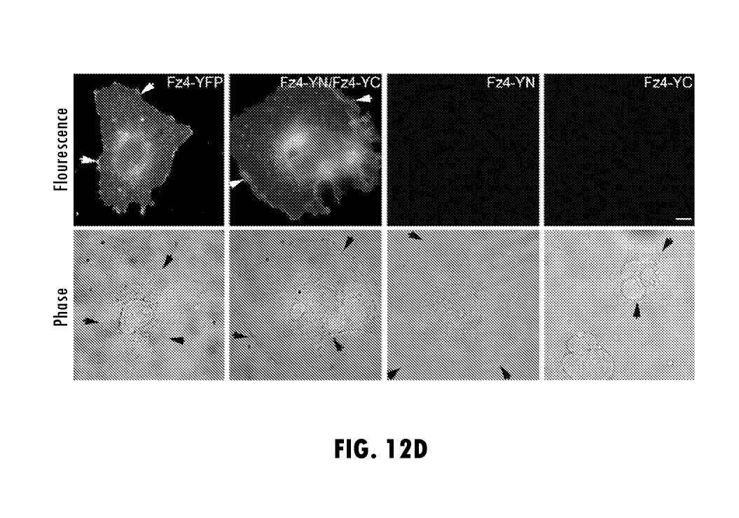

Based on the Norrin dimeric structure, a model of Norrin activation of Fz4 through ligand-induced Fz4 receptor dimerization was initially favored. To determine whether one Norrin dimer can bind two Fz4 CRDs, MBP-Norrin was incubated with two Fz4 CRD proteins, each tagged with either a T4 lysozyme and v5H6 fusion tag (T4L-v5H6) or a FcH6 tag. Fz4 CRD-FcH6 coimmunoprecipitated with Fz4 CRD-T4Lv5H6 in the presence, but not the absence, of MBP-Norrin, demonstrating that each Norrin dimer indeed binds to two Fz4 CRD domains (FIG. 2F). Next bioluminescence resonance energy transfer (BRET) (Harikumar et al. 2007) was used to examine the effect of Norrin on Fz4 full-length receptor dimerization, using Fz4 receptors tagged with yellow fluorescent protein (YFP) or Renilla luciferase (Rlu). As controls, Fz4 surface expression was observed when cells were transfected with either YFP- or Rlu-tagged Fz4 (FIG. 12A). Full-length Fz4 receptors surprising interacted strongly with each other in the absence of Norrin, producing a BRET signal as strong as that of the positive control, a covalent Rlu-YFP fusion protein (FIG. 3A). To further distinguish genuine receptor interaction from random collision, saturation BRET (Harikumar et al. 2007) was performed. Coexpression of a constant amount of Fz4-Rlu with increasing amounts of Fz4-YFP generated a hyperbolic, plateau-reaching signal, indicative of a true interaction, whereas titrating Fz4-Rlu with CCK2R-YFP, an unrelated seven-transmembrane receptor, generated a non-saturating, quasi-linear signal, indicative of random collisions (FIG. 3B). These data suggest that the Fz4 receptor exists as a dimer in the absence of exogenous ligands. Interestingly, the human protein smoothened, a remote homolog of Fz4, also homodimerizes through its transmembrane domain (Wang et al. 2013).

The effect of recombinant MBP-Norrin on Fz4 dimerization was then examined and found that Norrin did not further increase the Fz4 BRET signal; on the contrary, it slightly but reproducibly reduced the signal (FIG. 3C). Cotransfection of Norrin with YFP- and Rlu-tagged Fz4 expression constructs resulted in a similar small signal decrease (FIG. 3C). This effect was not observed for Norrin R41E, which does not bind to Fz4 (Smallwood et al. 2007), indicating that the BRET decrease depends on Norrin-Fz4 binding (FIG. 3C). Since BRET signals are exquisitely sensitive to small changes in donor-receptor distance and relative donor and acceptor orientations, the signal reduction in the presence of Norrin could indicate that Norrin induces a slight increase in the distance between the Fz4 receptor monomers or a conformational change of Fz4 receptors, but not dissociation of Fz4 dimer. In control experiments, Norrin changed neither the total expression nor the surface expression of Fz4 (FIGS. 12B, and 12C). Together, these data suggest that Fz4 exists as dimers in the absence of Norrin and that Norrin binding neither increases nor disrupts Fz4 dimer formation.

To examine whether Fz4 exists as higher-order oligomers in the presence or absence of Norrin, a split-YFP BRET assay (Harikumar et al. 2008a) was used that contained three components: one Fz4 receptor tagged with Rlu (Fz4-Rlu), one Fz4 receptor tagged with an N-terminal fragment of YFP (Fz4-YFP-N), and one Fz4 receptor tagged with a C-terminal fragment of YFP (Fz4-YFP-C). Neither of the two YFP fragments fluoresces on their own, but the two fragments form a functional YFP when brought into close proximity. As controls, Fz4 surface expression was observed when cells were transfected with either YFP-N- or YFP-C-tagged Fz4 (FIG. 12A) and observed YFP fluorescence when cells were transfected with both YFP-N- and YFP-C-tagged Fz4 (FIG. 3D (1-3), FIG. 12D), confirming the BRET results for constitutive Fz4 dimer formation. However, regardless of the presence or absence of Norrin, the three-component BRET assay showed no BRET signal above background (FIG. 3E). Similarly, Fz4-Rlu, Fz4-YFP-N, and Fz4-YFP-C did not produce a clear BRET signal in the saturation BRET assay (FIG. 3F). Together, the data suggests that: 1) Fz4 exists as constitutive dimers regardless of the presence of Norrin, which is consistent with a previous report (Kaykas et al. 2004); 2) Fz4 dimerization itself is not sufficient for receptor activation; and 3) Norrin does not activate the Fz4 receptor by inducing Fz4 dimerization. Based on the three-component BRET experiments, Fz4 likely does not form higher-order oligomers. However, it cannot be excluded that Fz4 can possibly form transient oligomers or oligomers having special geometry that cannot be detected by the BRET assay.

### F. Norrin Binds to the β-Propeller 1 and 2 Fragments of Lrp6 Protein

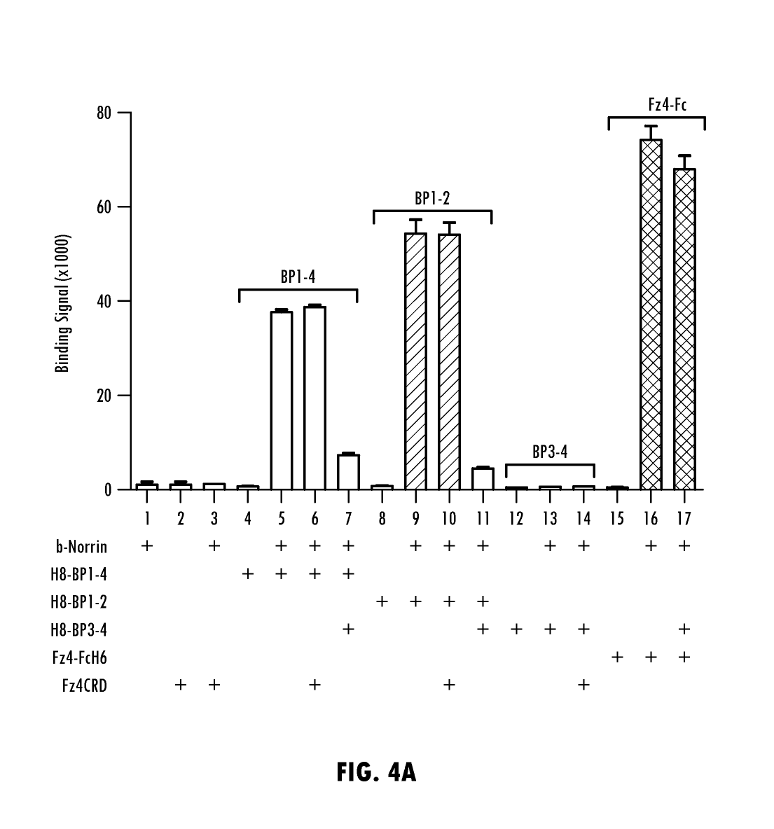

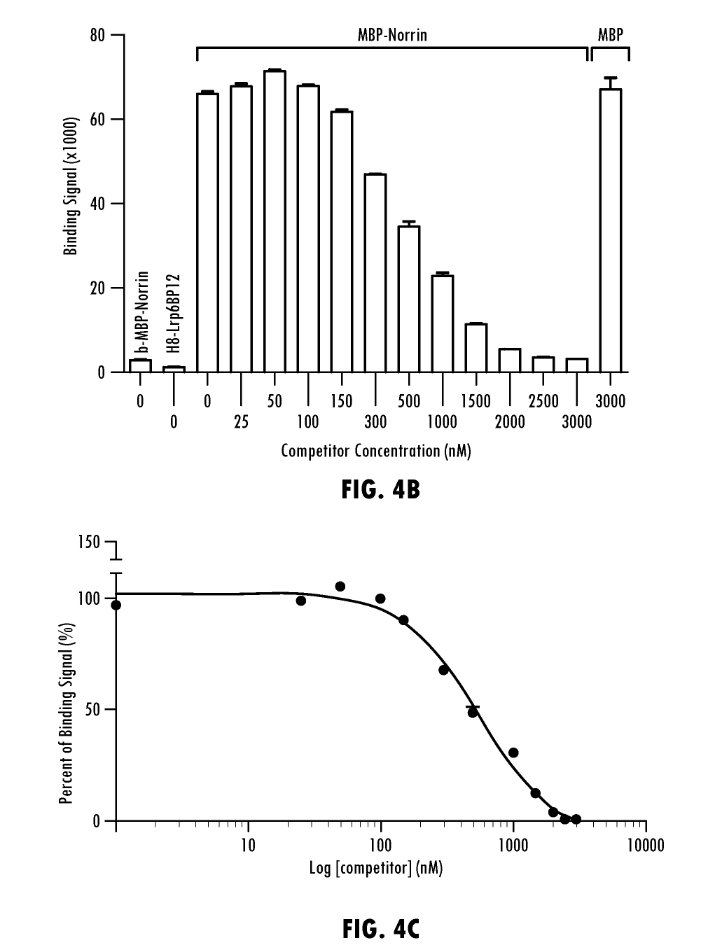

From genetic studies, Lrp5 is known to be functionally involved in the Norrin/Fz4 signaling complex (Gong et al. 2001), yet previous work has failed to identify a direct interaction between Lrp5/Lrp6 and Norrin fused to alkaline phosphatase (Xu et al. 2004). Purified proteins were used to examine direct interactions between the MBP-Norrin and Lrp6 ECD using the highly sensitive AlphaScreen assay. The Lrp6 ECD contains four β-propeller domains (BP1-4). Biotinylated MBP-Norrin interacted with Lrp6 β-propeller domains 1-4 (BP1-4) and BP1-2, but not BP3-4 (FIG. 4A, lanes 5 and 9). The interaction between biotinylated MBP-Norrin and Lrp6 BP1-2 can be competed with excess unlabeled MBP-Norrin, but not with MBP, with an IC50 of approximately 570 nM and Kd of approximately 450 nM using a homologous competition assay (FIG. 4B, 4C).

Interestingly, when BP3-4 was added to MBP-Norrin plus either BP1-2 or BP1-4, BP3-4 was found to greatly reduce the binding signals (FIG. 4A, lanes 5 vs. 7 and 9 vs. 11), whereas adding Fz4 CRD did not compete their interaction (FIG. 4A, Lanes 5 vs. 6 and 9 vs. 10). The ability of BP3-4 to inhibit the interaction between BP1-4/BP1-2 and Norrin is consistent with an observed interaction between BP3-4 and BP1-2 (Liu et al. 2003), and suggests that this interaction interferes with BP12 and MBP-Norrin. Because adding Fz4 CRD protein did not interfere with the interaction between MBP-Norrin and Lrp6 BP1-2 (FIG. 4A, lane 9 vs. 10), and Lrp6 BP3-4 did not inhibit the interaction between MBP-Norrin and Fz4-FcH6 (FIG. 4A, lanes 16 vs 17), Lrp6 BP1-2 and Fz4 CRD bind to different regions of Norrin.

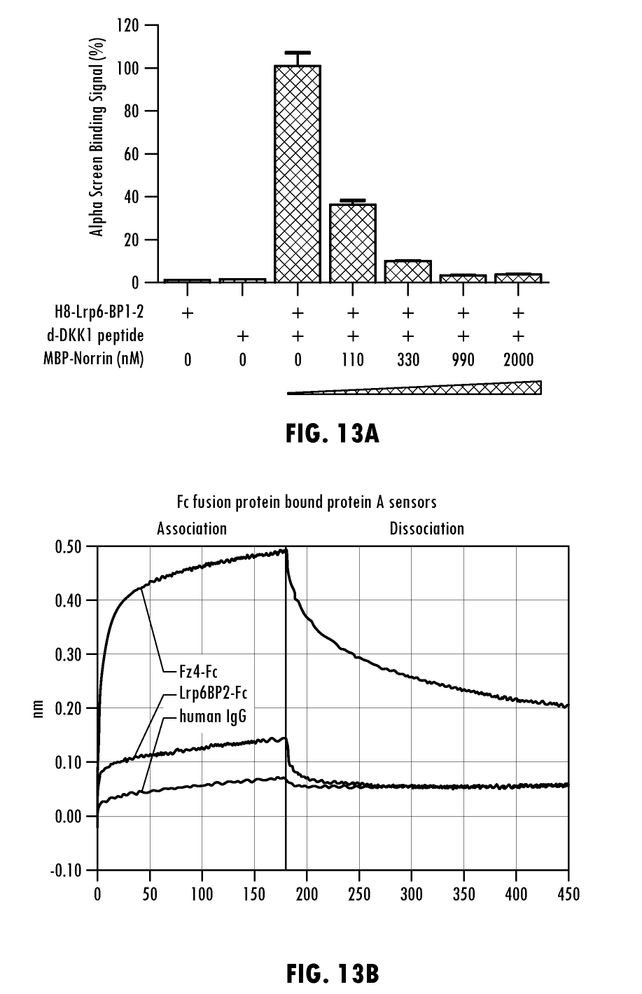

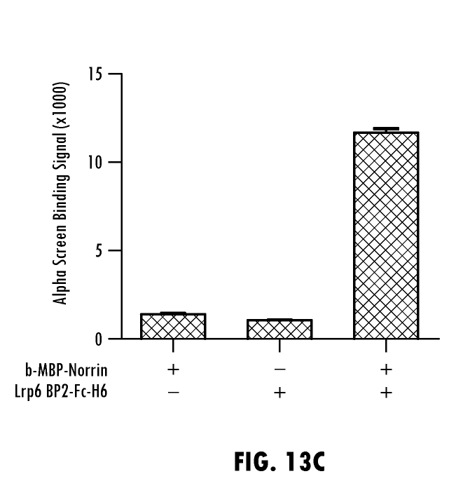

The peptide motif Asn-Ala-Ile-Lys within DKK1 and several other Wnt inhibitors specifically binds to the Lrp6 BP1 domain (Bourhis et al. 2011). The interaction between biotinylated DKK1 peptide and His8-tagged Lrp6 BP1-2 was confirmed using an AlphaScreen assay (FIG. 13A, Lane 3) and thus MBP-Norrin competes in this interaction with an IC₅₀ of 72 nM (FIG. 13A, Table 3).

TABLE 3
IC₅₀ values obtained by using purified MBP-Norrin wild-type and
mutant proteins to compete the Fz4-Fc-H6 and biotin-MBP-Norrin
interaction or H8-Lrp6 BP1-2 and biotin DKK1 peptide interaction.

| | IC₅₀ (nM) | |
|---|---|---|
| Competition with | Fz4-CRD interaction | Lrp6 BP1-2 interaction |
| Wild-type | 34 | 72 |
| R41E | NC | 288 |
| K54E/R109E | 18 | NC |
| C55A/C110A | 37 | 207 |

NC stands for no competition.

This suggests that MBP-Norrin binds to a site on Lrp6 BP1 that overlaps with the binding site of Wnt inhibitors. To test whether Norrin can also bind to BP2, we purified BP2 as an Fc fusion protein and measured its binding to MBP-Norrin by biolayer interferometry. Protein A sensors specifically bind to the Fc region of human IgG. Lrp6 BP2-FcH6 bound to a protein A sensor interacted with MBP-Norrin with moderate affinity, whereas human IgG-bound protein A sensor failed to interact (FIG. 13B). The binding between biotinylated MBP-Norrin and BP2-FcH6 was also confirmed by AlphaScreen assay (FIG. 13C). Although we were unable to purify isolated BP1 as a stable protein, the BP2 binding data and the Norrin competition of DKK1 peptide binding to BP1 suggest that the Norrin dimer is capable of binding to both the BP1 and BP2 domains of Lrp6.

### G. Norrin Contains Separate Binding Sites for Lrp6 BP1-2 and Fz4 CRD

If Norrin has separate binding sites for Lrp5/6 and Fz4, then two classes of Norrin mutations should exist that selectively disrupt those interactions. Indeed, two classes of Norrin mutations have been identified by systematic mutagenesis of Norrin (Smallwood et al. 2007). One class of mutations, including R41E, H43A/Y44A/V45A, M59A/V60A/L61A, and Y120A/R121A/Y122A, disrupted Norrin binding to the Fz4 CRD and strongly reduced the ability of Norrin to activate the TCF reporter. Mapping these mutations onto the Norrin structure identified a continuous surface area consisting of the R41, H43, V45, L61, Y120, and Y122 residues (FIG. 5A). This surface is exposed and is not at the dimer interface, and therefore is likely the major binding site for Fz4. The other class of mutations, including L52A/Y53A/K54A, K54E, R107E, R109E, and R115E, did not affect Fz4 binding, yet reduced the ability of Norrin to activate the TCF reporter. These residues are clustered on the edge of the Norrin molecule in the β1-β2 and β3-β4 loop regions that are linked through the C55-C110 disulfide bond (FIG. 5A). Interestingly, the C55A/C110A mutation, which did not affect Fz4 binding, also reduced the ability of Norrin to activate the TCF reporter (Smallwood et al. 2007). We hypothesize that these residues reduce Norrin signaling activity by reducing or disrupting Norrin binding to Lrp6.

To test our hypotheses, we engineered a number of Norrin mutations and tested their abilities to activate the TCF reporter in the presence of Fz4 and Lrp6. Mutations that interfered with Fz4 binding (R41E, H43A/V45A, L61A, and Y120A/Y122A) or putative Lrp5/6 binding (K54E, R107E, R109E, K54E/R109E, and C55A/C110A) reduced Norrin's signaling activity (FIG. 5B). We then purified three Norrin mutant proteins (R41E, K54E/R109E, and C55A/C110A) to test their binding to Fz4 and Lrp6, respectively. The unlabeled wild-type MBP-Norrin, as well as the C55A/C110A and K54E/R109E mutant proteins, all efficiently competed the Fz4-binding signal with IC50 values of 34 nM, 37 nM, and 18 nM, respectively (Table 3). In contrast, the R41E mutant was unable to compete the Fz4-binding signal, indicating that it is severely compromised in Fz4 binding (FIG. 5C).

The ability of these three mutant proteins to bind Lrp6 BP1-2 was tested by quantitating their competition for the interaction between His8-tagged Lrp6 BP1-2 and the biotin-DKK1 peptide. Wild-type MBP-Norrin competed the interaction with an IC50 of 72 nM, while the C55A/C110A and R41E mutants competed with an IC₅₀ of 207 nM and 288 nM, respectively (Table 3), indicating that both mutants are partially compromised in binding to Lrp6 BP1-2. In contrast, the K54E/R109E double mutant was not able to compete the binding between the DKK1 peptide and Lrp6 BP1-2, indicating its inability to bind to Lrp6 (FIG. 5D). Together, these data support the hypothesis that Norrin contains two separate binding sites: one site, in part comprised of residue R41, is for binding of Fz4; the other site, comprised of residues K54, C55, R109, and C110, is for the binding of Lrp6. When mapped on the structure, these two binding sites appear to be adjacent but not overlapping (FIG. 5A).

### H. Norrin, Lrp6 BP1-2, and Fz4 CRD Form a Ternary Complex

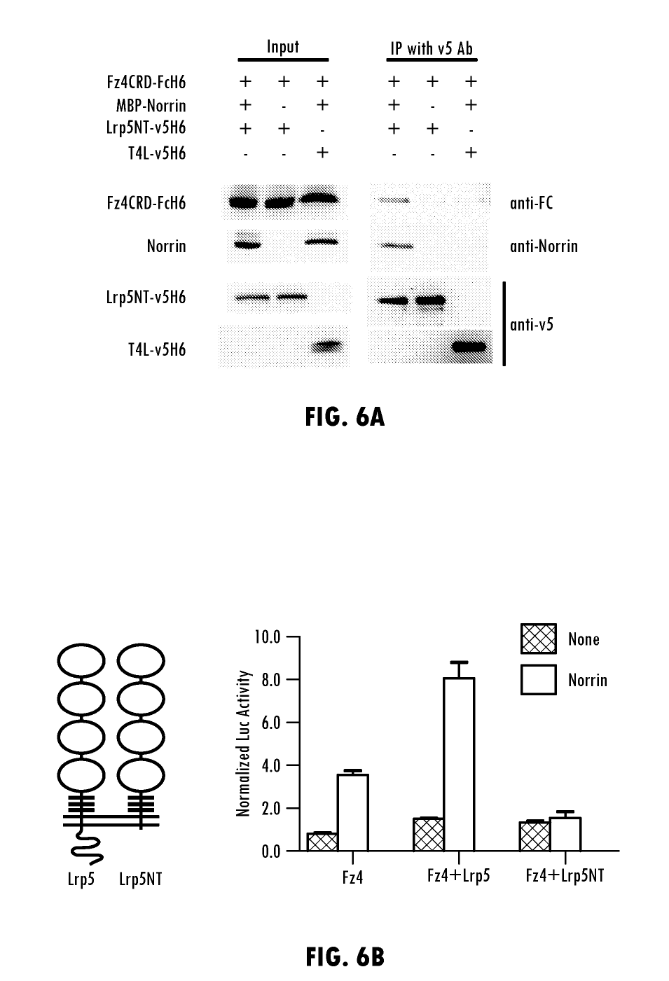

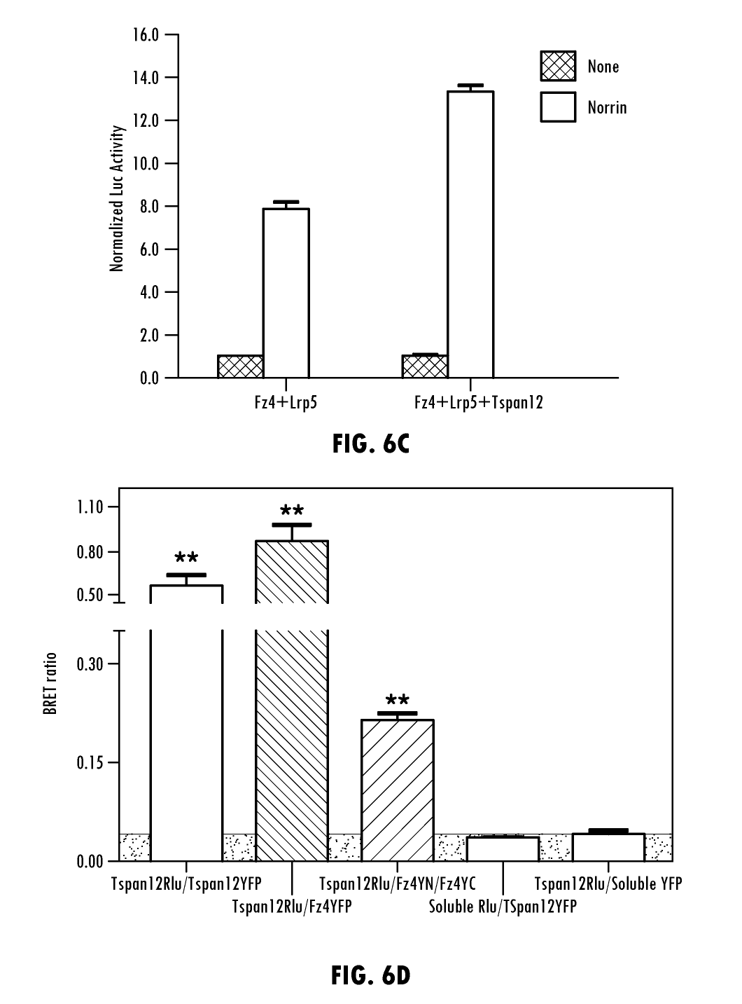

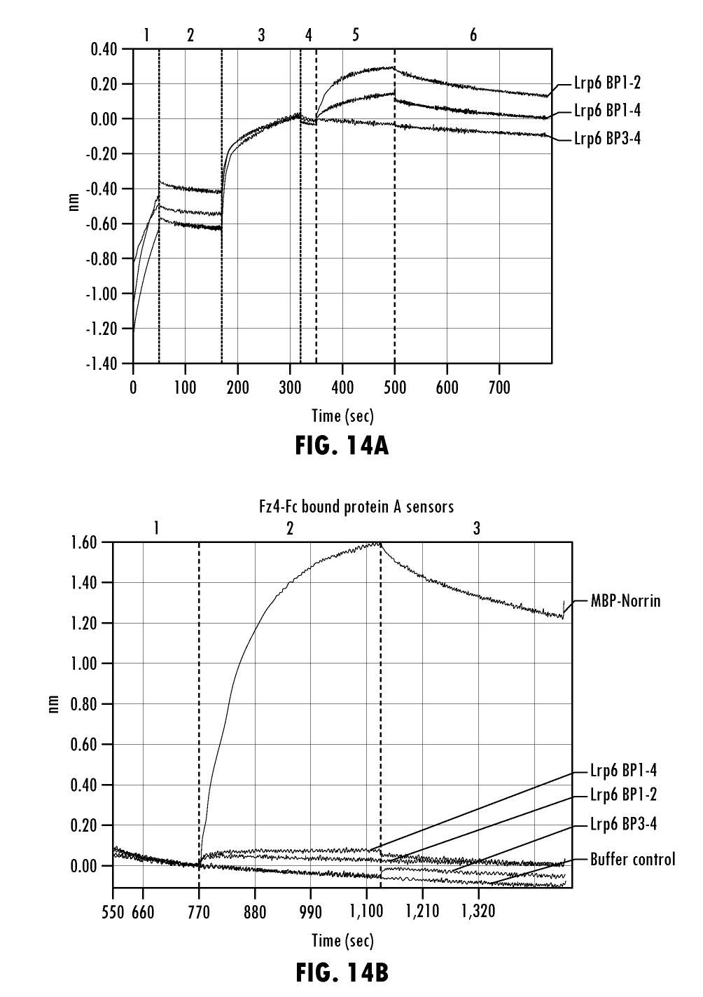

The two binding sites on Norrin for Lrp6 and Fz4 suggest that they may form a ternary complex. To test complex formation, Fz4-FcH6, MBP-Norrin, and Lrp5NT (which contains the ECD and TM domains of Lrp5 fused to a v5H6 tandem tag) was expressed. When Lrp5NT protein was immunoprecipitated with v5 antibody, Fz4-FcH6 coimmunoprecipitated in the presence, but not in the absence, of MBP-Norrin (FIG. 6A), supporting formation of a ternary Lrp5-Norrin-Fz4 complex in which MBP-Norrin interacts with both Lrp5 ECD and Fz4 CRD. These results were confirmed with a three-hybrid biolayer interferometry assay. When Fz4-FcH6-bound biosensors were first incubated with MBP-Norrin and then with Lrp6 BP1-2, Lrp6 BP1-2 was able to further bind to MBP-Norrin pre-bound to Fz4-FcH6 (FIG. 14A). Fz4-bound MBP-Norrin also interacted weakly with Lpr6 BP1-4, but not with BP3-4. As controls, the Fz4 CRD did not significantly interact with any of the Lrp6 ECD fragments (FIG. 14B), while MBP-Norrin could interact with both the Fz4 CRD (FIGS. 1C, 1D) and the Lrp6 ECD (FIG. 4A). These data indicate that Norrin can bind simultaneously to Fz4 CRD and Lrp6 BP1-2 to induce the formation of a ternary complex.

### I. The Roles of Lrp5/6 and Tspan12 in Norrin Mediated β-Catenin Signaling

To examine the functional importance of Lrp5 for Norrin signaling, we tested whether Lrp5-NT, which lacks the intracellular domain (FIG. 6B), could interfere with Norrin-mediated signaling. Previous studies showed that Lrp5-NT acts as a dominant negative inhibitor of the Wnt/β-catenin signaling pathway (Tamai et al. 2000), presumably because Lrp5-NT is insufficient for signaling, but competes with endogenous Lrp5/6 for Wnt ligand binding. As positive controls, Norrin activated the TCF reporter in 293STF cells transfected with Fz4, and cotransfection with Lrp5 further enhanced the reporter activity (FIG. 6B). Due to ubiquitous expression of endogenous Lrp5 and Lrp6, these cotransfection experiments cannot distinguish between either a stimulatory or an essential function of Lrp5 in Norrin/Fz4-mediated signaling. However, the complete loss of responsiveness to Norrin in cells expressing Fz4, endogenous Lrp5/6, and high levels of Lrp5-NT (FIG. 6B) suggests that the Lrp5-N domain competes for a functionally critical interaction between Norrin and endogenous Lrp5/6, and that Lrp5/6 is essential for Norrin-mediated Wnt/β-catenin signaling.

Tspan12 is an additional factor that is specifically involved in Norrin/Fz4/Lrp5 signaling (Junge et al. 2009). Using cell-based assays, the finding that cotransfection with Tspan12 further increased Norrin/Fz4/Lrp5-mediated TCF reporter activity (FIG. 6C) was confirmed. Using the BRET assay, we observed very strong BRET signals from Tspan12-Rlu-Tspan12-YFP and Tspan12-Rlu-Fz4-YFP pairs (FIG. 6D), indicating that Tspan12 can both homodimerize and heterodimerize with Fz4. Interestingly, Tspan12-Rlu can form a ternary complex with Fz4-YFP-N and Fz4-YFP-C in a split-YFP BRET assay (FIG. 6D). This suggests that Tspan12 and Fz4 interactions do not disrupt the Fz4-Fz4 dimer interactions. The experiments performed herein are consistent with previous experiments showing that Tspan12 interacts specifically with Fz4 to facilitate Norrin mediated β-catenin signaling (Junge et al. 2009).

### Example 3

### Discussion

### A. Discussion

The expression and purification of Norrin has been a major obstacle that has hampered its structural studies. In this application, experiments were performed with the aim of developing a new method for expression and purification of Norrin, which eventually led the inventors to solve its crystal structure. Norrin has a novel dimeric structure, and we were able to map two separate sites on the surface of Norrin, one for binding to Fz4 receptor and one for binding to Lrp5/6 co-receptor, which helped us establish a model of an activation complex consisting of Norrin, Fz4, Lrp5/6 and Tspan12. These results provide important insights into molecular mechanisms of Norrin signaling and unify the roles of Lrp5/6 as co-receptors in both Wnt and Norrin signaling.

### B. Norrin Dimeric Structure

The overall structure feature of Norrin is a homodimer linked through three intermolecular disulfide bonds. This structure is very different from the recent structure of Wnt8 in complex with the Fz8 CRD, which has a monomeric structure with a central “palm” domain and an extended “thumb” and “index finger” (Janda et al. 2012). Importantly, Norrin lacks any lipid modification, which is present in all Wnt proteins and is required for their function (Nusse 2003; Takada et al. 2006). This suggests that the binding between Norrin and Fz4 receptor is not driven by lipid mediated hydrophobic interactions, but through specific protein-protein interactions.

Norrin belongs to the cystine knot growth factor superfamily, and dimer formation is a common theme for these growth factors (Sun and Davies 1995). Consistent with a stable dimer structure, Norrin has extensive interactions at the dimer interface, including disulfide bonds, hydrogen bonds, and hydrophobic interactions (FIG. 2C-E). Although the monomeric structure of Norrin contains the conserved cystine knot motif, the dimeric structure of Norrin is different from that of other cystine knot proteins. For instance, NGF forms a dimer in a head-to-head orientation, whereas TGF-β3 and PDGF-BB form a dimer in a head-to-tail orientation. NGF, TGF-β3, and PDGF-BB all form homodimers through zero, one, and two intermolecular disulfide bonds, respectively. In contrast, Norrin forms a homodimer in a head-to-tail fashion with a unique semi-circular shape linked by three intermolecular disulfide bonds (FIG. 10B). The importance of the Norrin dimer is supported by the functional consequences of mutations that disrupt the three disulfide bonds or the hydrophobic interactions at the dimer interface (FIGS. 11B, 11C).

### C. Mapping Fz4 and Lrp5/6 Binding Sites on the Norrin Dimeric Structure

Mapping previous Norrin mutations (Smallwood et al. 2007) onto the current Norrin structure, we identified an exposed surface region, including amino acids R41, H43, V45, L61, Y120, and Y122, as the Fz4-binding site (FIG. 5A). Mutations in these residues significantly reduced Fz4 binding and Norrin signaling activity (FIGS. 5B, 5C). As shown by sequence alignment (FIG. 15A), all these residues are conserved across species. The fact that a Norrin dimer contains two symmetric Fz4-binding sites suggests that the dimer can bind to two Fz4 CRD domains, which was confirmed by immunoprecipitation (FIG. 2F). Two FEVR mutations were identified in the Fz4 CRD domain (M157V and M105V), which indicates that the base of the Fz4 CRD domain including these two residues is the surface for Norrin interaction (Xu et al. 2004).

In addition, we identified the Lrp5/6 binding site on the edge of Norrin, which comprises several positively charged residues (K54, R107, R109, and R115) and two hydrophobic residues (L52 and Y53) (FIG. 5A), and which specifically binds to the BP1-2 domain (FIG. 5D). Mutation of these residues reduced signaling activity but did not affect Fz4 binding (FIG. 5C) (Smallwood et al. 2007). The sequence alignment showed that all the positively charged residues are completely conserved and the hydrophobic residues L52 and Y53 are largely conserved (FIG. 15A). The top concave surfaces of Lrp6 BP1 and BP2 contain a hydrophobic patch surrounded by negatively charged residues (Cheng et al. 2011). The hydrophobic and positively charged residues of Norrin may thus interact with the hydrophobic patch and the negatively charged residues on the top surface of BP1 or BP2.

### D. Assembly and Activation of Norrin-Fz4-Lrp5/6-Tspan12 Signaling Complex

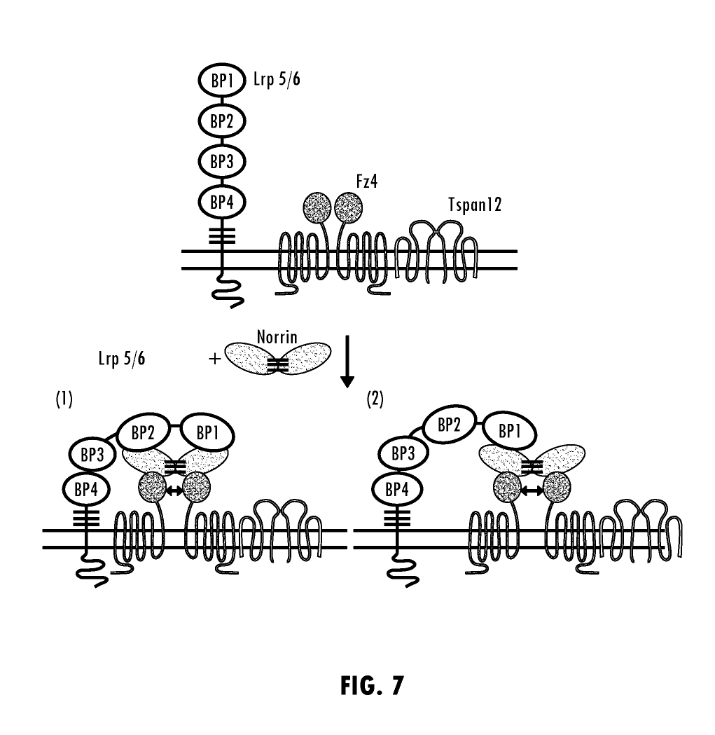

The BRET and split-YFP data showed that Fz4 preexists as dimers on the cell membrane (FIGS. 3A, 3D). Because Norrin also forms stable dimers, Norrin binds Fz4 with a 2:2 stoichiometry. We showed that Norrin binds to Lrp6 BP1-2 in addition to its binding to Fz4 (FIG. 4A). Previous work failed to detect an interaction between Norrin and Lrp5/6, which was likely due to the weak binding of Norrin to Lrp5/6 BP1-2 (affinity more than 40-fold lower than Norrin-Fz4 interactions). The Norrin homodimer with two symmetric edges can bind to one BP1-2 or two BP1-2 molecules (FIG. 7). Because a Norrin dimer can bind to both BP1 and BP2 domains of Lrp6 (FIG. 13A-13C), we favor a model where one Norrin dimer binds to one Lrp5/6 monomer, with each Norrin monomer binding to one BP domain of Lrp5/6 (FIG. 7). Synergistic binding of Norrin to both the BP1 and BP2 domains would further stabilize Norrin dimerization, consistent with the ability of Lrp5 and Lrp6 to partially rescue dimerization-compromised Norrin mutants (11B, 11C), although we cannot exclude the possibility that one Norrin dimer can interact with two Lrp5/6 co-receptors.

The finding that the Lrp6 BP3-4 fragment failed to bind Norrin, yet competed the interaction between Norrin and Lrp6 BP1-4/BP1-2 suggests that Lrp6 BP3-4 may intermolecularly interact with BP1-4/BP1-2. It is known that the Lrp5/6 ECD domain has an inhibitory role in basal Lrp5/6 activity (Mao et al. 2001a; Mao et al. 2001b). It was also reported that Lrp6 forms an inactive dimer mediated through the extracellular BP domains (Liu et al. 2003). Based on these studies, Lrp6 may form homodimers (a closed conformation) through BP12-BP12 or BP12-BP34 interactions. The direct binding of Norrin to Lrp6 BP1-2 would disrupt or weaken the interaction between Lrp6 dimers, thus releasing the autoinhibitory conformation of Lrp6.

Importantly, functional Lrp5/6 coreceptor is required for Norrin/Fz4 signaling (FIG. 6B) and that Norrin forms a ternary complex with both the Fz4 CRD and Lrp6 ECD in vitro (FIG. 6A, FIG. 14A). This suggests that the signaling mechanism for Norrin is likely very similar to that of canonical Wnts by inducing heterodimerization of Fz4 with Lrp5/6 (Cong et al. 2004). Furthermore, Fz4 activation by Norrin is enhanced by Tspan12, which specifically interacts with Fz4 (FIG. 6D), but not with other Fz proteins such as Fz5 (Junge et al. 2009). We speculate that Tspan12 is a molecular chaperone that stabilizes Fz4 protein to facilitate Norrin interaction and signaling. Tspan12, by forming heterodimers with Fz4, may compartmentalize Fz4 into tetraspanin-enriched microdomains on the cell membrane (Bailey et al. 2011) to facilitate Norrin/Fz4-mediated β-catenin signaling.

### E. Structural Basis of Norrie Disease Mutations

In spite of its small size, more than 100 disease-causing Norrin mutations have been identified in human patients (Ye et al. 2010). The Norrin structure provided an opportunity to examine the molecular basis of disease-causing missense mutations (Table 4 and FIG. 10C).

TABLE 4
Known missense mutations in Norrie disease, related to FIG. 2A and FIG.
10C. Mutations were categorized into six groups based on our structural and functional
studies of Norrin (Cysteine mutations, Dimer interface mutations, Hydrophobic packing
and protein stability mutations, Fz4 binding site mutations, Lrp5/6 binding site mutations,
and other mutations). The full names of the disease acronyms are shown below the table.
The information for Norrin mutations is obtained from the website
(http://www.medmolgen.uzh.ch/research/eyediseases/norriedisease/Norrinmutations.html).

| | Mutation | Disease | | Mutation | Disease |
|---|---|---|---|---|---|
| Cysteine | C39R | ND | Dimer Interface | Y44C | ND |
| mutations | C55R, C55F | ND | | L62P | ND |
| | C65Y, C65W | ND | | A63D, A63S | ND |
| | C69S | ND | | E66K | PFV |
| | C95R, C95S | ND | | G67R, G67E | ND |
| | | (severe) | | | |
| | C96Y | ND, EVR | | R74C | ND, FEVR |
| | C96W | CD | | S75P, S75C | ND |
| | C110R, C110S | ND | | F89L | ND |
| | C110G | FEVR | | R97P | ND |
| | C126S | ND | | P98L | ND |
| | C128R | ND | | L108P | ROP |
| Fz4 | R41K | EVR | | A118D | ND |
| binding | R41T | ND | | Y120C | EVR |
| site | R41S | PFV | | R121G | ND, PRDX |
| | H43R, H43Q | ND | | R121W | ND, FEVR, ROP |
| | V45M, V45E | ND | | R121Q | ND, FEVR |
| | L61F | ND, VI | | R121L | FEVR |
| | L61I | FEVR | | I123N | ND |
| | L61P | ND | Hydrophobic | V60E | ND |
| | L124F | FEVR | packing and | R90C, R90P | ND |
| Lp5/6 | K54N | ND, | protein stability | L103V | FEVR |
| binding | | FEVR | | | |
| site | R115L | FEVR | | L108P | ROP |
| | K104Q | ND | | Y120C | EVR |
| | | (mild) | | | |
| | K104N | ND | | I123N | ND |
| | | | | S92P | ND |
| | | | | S101F | ND, PHPV |
| | | | | A105T | ND |

ND = Norrie Disease,
(F)EVR = (Familial) Exudative Vitreoretinopathy,
PFV = Persistent Fetal Vasculature,
ROP = Retinopathy Of Prematurity,
CD = Coats Disease,
PRDX = Primary Retinal Dysplasia,
VI = Venous Insufficiency,
PHPV = Primary Hyperplastic Persistent Vitreous

Based on the structure, the majority of these mutations can be categorized into five different groups: cysteine residue mutations, dimer interface mutations, hydrophobic core mutations, Fz4 binding site mutations, and Lrp5/6 binding site mutations (Table 4). The Norrin structure readily explains these disease-causing mutations, as they would compromise protein functions by affecting protein folding and stability, dimerization, or functional interaction with Fz4 and Lrp5/6. The large number of disease-causing mutations in Norrin suggest that this protein is very sensitive to perturbations, which is consistent with the multiple functions (protein folding, dimerization, receptor and co-receptor binding) that it mediates.

### F. The Norrin Structure May Serve as Model for Mucins and Von Willebrand Factor.

In a new sequence homology search, Norrin was found to be most closely related to the C-terminal domains of mucins and von Willebrand factor, two extracellular proteins with multiple domain structures. While only 7 of the 11 cysteines in Norrin are conserved in TGF-β, all 11 cysteines are conserved in mucins and von Willebrand factor (FIG. 15B). The structure of Norrin may therefore also represent that of mucins and von Willebrand factor C-terminal domains. In particular, the C55-C110 disulfide bond is conserved in Norrin, mucins, and von Willebrand factor (blue stars, FIG. 15B), whereas TGF-β proteins have a unique disulfide bond that is not found in those three proteins (green stars, FIG. 15B). Moreover, the three-disulfide-bond linked dimer is a conserved feature of these proteins (red stars, FIG. 15B) and mutational studies of pig submaxillary mucin also identified three cysteines that may be involved in forming the intermolecular disulfide bonds of the mucin dimer (Perez-Vilar and Hill 1998). Both mucins and von Willebrand factor can modulate Wnt signaling through interaction with other proteins (Rey and Ellies 2010). These domains in mucins and von Willebrand factor could function as a dimerization and signaling domain, for which receptors remain to be identified.

In summary, the inventors have determined the crystal structure of Norrin, which revealed a stable dimer with three intermolecular disulfide bonds. Structure-based mapping revealed separate sites on the Norrin surface for binding of Fz4 and Lrp5/6, and Norrin forms a ternary complex with Fz4 CRD and Lrp6 ECD in vitro. The inventors propose that dimeric Norrin activates Fz4 by inducing heterodimerization of Fz4 with Lrp5/6, and it may also induce conformational changes in the Fz4-Lrp5 signaling complex. These results provide important mechanistic insights into the assembly and activation of the Norrin-Fz4-Lrp5/6-Tspan12 signaling complex and unify the roles of Lrp5/6 as the common co-receptors for both Wnt and Norrin signaling.

### REFERENCES

Bailey R L, Herbert J M, Khan K, Heath V L, Bicknell R, Tomlinson M G. 2011. The emerging role of tetraspanin microdomains on endothelial cells. Biochem Soc Trans 39: 1667-1673.
Berger W. 1998. Molecular dissection of Norrie disease. Acta Anat (Basel) 162: 95-100.
Bourhis E, Wang W, Tam C, Hwang J, Zhang Y, Spittler D, Huang O W, Gong Y, Estevez A, Zilberleyb I et al. 2011. Wnt Antagonists Bind through a Short Peptide to the First beta-Propeller Domain of LRP5/6. Structure 19: 1433-1442.
Bradford M M. 1976. A rapid and sensitive method for the quantitation of microgram quantities of protein utilizing the principle of protein-dye binding. Anal Biochem 72: 248-254.
Chen Z Y, Battinelli E M, Fielder A, Bundey S, Sims K, Breakefield X O, Craig I W. 1993. A mutation in the Norrie disease gene (NDP) associated with X-linked familial exudative vitreoretinopathy. Nat Genet 5: 180-183.
Cheng Y, Prusoff W H. 1973. Relationship between the inhibition constant (K1) and the concentration of inhibitor which causes 50 percent inhibition (I50) of an enzymatic reaction. Biochem Pharmacol 22: 3099-3108.
Cheng Z, Biechele T, Wei Z, Morrone S, Moon R T, Wang L, Xu W. 2011. Crystal structures of the extracellular domain of LRP6 and its complex with DKK1. Nat Struct Mol Biol.
Cong F, Schweizer L, Varmus H. 2004. Wnt signals across the plasma membrane to activate the beta-catenin pathway by forming oligomers containing its receptors, Frizzled and LRP. Development 131: 5103-5115.
Emsley P, Cowtan K. 2004. Coot: model-building tools for molecular graphics. Acta Crystallogr D Biol Crystallogr 60: 2126-2132.
Gong Y, Slee R B, Fukai N, Rawadi G, Roman-Roman S, Reginato A M, Wang H, Cundy T, Glorieux F H, Lev D et al. 2001. LDL receptor-related protein 5 (LRP5) affects bone accrual and eye development. Cell 107: 513-523.
Harikumar K G, Happs R M, Miller L J. 2008a. Dimerization in the absence of higher-order oligomerization of the G protein-coupled secretin receptor. Biochim Biophys Acta 1778: 2555-2563.
Harikumar K G, Morfis M M, Sexton P M, Miller L J. 2008b. Pattern of intra-family hetero-oligomerization involving the G-protein-coupled secretin receptor. J Mol Neurosci 36: 279-285.
Harikumar K G, Pinon D I, Miller L J. 2007. Transmembrane segment I V contributes a functionally important interface for oligomerization of the Class II G protein-coupled secretin receptor. J Biol Chem 282: 30363-30372.
He X, Semenov M, Tamai K, Zeng X. 2004. LDL receptor-related proteins 5 and 6 in Wnt/beta-catenin signaling: arrows point the way. Development 131: 1663-1677.
Holmen S L, Robertson S A, Zylstra C R, Williams B O. 2005. Wnt-independent activation of beta-catenin mediated by a Dkk1-Fz5 fusion protein. Biochem Biophys Res Commun 328: 533-539.
Huelsken J, Behrens J. 2002. The Wnt signalling pathway. J Cell Sci 115: 3977-3978.
Janda C Y, Waghray D, Levin A M, Thomas C, Garcia K C. 2012. Structural basis of Wnt recognition by Frizzled Science 337: 59-64.
Junge H J, Yang S, Burton J B, Paes K, Shu X, French D M, Costa M, Rice D S, Ye W. 2009. TSPAN12 regulates retinal vascular development by promoting Norrin—but not Wnt-induced FZD4/beta-catenin signaling. Cell 139: 299-311.
Kaykas A, Yang-Snyder J, Heroux M, Shah K V, Bouvier M, Moon R T. 2004. Mutant Frizzled 4 associated with vitreoretinopathy traps wild-type Frizzled in the endoplasmic reticulum by oligomerization. Nat Cell Biol 6: 52-58.
Ke J, Chelvarajan R L, Sindhava V, Robertson D A, Lekakis L, Jennings C D, Bondada S. 2009. Anomalous constitutive Src kinase activity promotes B lymphoma survival and growth. Mol Cancer 8: 132.
Ke J, Zhang C, Harikumar K G, Zylstra-Diegel C R, Wang L, Mowry L E, Miller L J,
Williams B O, Xu H E. 2012. Modulation of beta-catenin signaling by glucagon receptor activation. PLoS One 7: e33676.
Liu G, Bafico A, Harris V K, Aaronson S A. 2003. A novel mechanism for Wnt activation of canonical signaling through the LRP6 receptor. Mol Cell Biol 23: 5825-5835.
Logan C Y, Nusse R. 2004. The Wnt signaling pathway in development and disease. Annu Rev Cell Dev Biol 20: 781-810.
Luhmann U F, Meunier D, Shi W, Luttges A, Pfarrer C, Fundele R, Berger W. 2005. Fetal loss in homozygous mutant Norrie disease mice: a new role of Norrin in reproduction. Genesis 42: 253-262.
Mao B, Wu W, Li Y, Hoppe D, Stannek P, Glinka A, Niehrs C. 2001a. LDL-receptor-related protein 6 is a receptor for Dickkopf proteins. Nature 411: 321-325.
Mao J, Wang J, Liu B, Pan W, Farr G H, 3rd, Flynn C, Yuan H, Takada S, Kimelman D, Li L et al. 2001b. Low-density lipoprotein receptor-related protein-5 binds to Axin and regulates the canonical Wnt signaling pathway. Mol Cell 7: 801-809.
Nusse R. 2003. Wnts and Hedgehogs: lipid-modified proteins and similarities in signaling mechanisms at the cell surface. Development 130: 5297-5305.
Ohlmann A, Seitz R, Braunger B, Seitz D, Bosl M R, Tamm E R. 2010. Norrin promotes vascular regrowth after oxygen-induced retinal vessel loss and suppresses retinopathy in mice. J Neurosci 30: 183-193.
Perez-Vilar J, Hill R L. 1997. Norrie disease protein (norrin) forms disulfide-linked oligomers associated with the extracellular matrix. J Biol Chem 272: 33410-33415.
Perez-Vilar J, Hill R L. 1998. The carboxyl-terminal 90 residues of porcine submaxillary mucin are sufficient for forming disulfide-bonded dimers. J Biol Chem 273: 6982-6988.
Pioszak A A, Parker N R, Suino-Powell K, Xu H E. 2008. Molecular recognition of corticotropin-releasing factor by its G-protein-coupled receptor CRFR1. J Biol Chem 283: 32900-32912.
Pioszak A A, Xu H E. 2008. Molecular recognition of parathyroid hormone by its G protein-coupled receptor. Proc Natl Acad Sci USA 105: 5034-5039.
Rehm H L, Zhang D S, Brown M C, Burgess B, Halpin C, Berger W, Morton C C, Corey D P, Chen Z Y. 2002. Vascular defects and sensorineural deafness in a mouse model of Norrie disease. J Neurosci 22: 4286-4292.
Rey J P, Ellies D L. 2010. Wnt modulators in the biotech pipeline. Dev Dyn 239: 102-114.
Seitz R, Hackl S, Seibuchner T, Tamm E R, Ohlmann A. 2010. Norrin mediates neuroprotective effects on retinal ganglion cells via activation of the Wnt/beta-catenin signaling pathway and the induction of neuroprotective growth factors in Muller cells. J Neurosci 30: 5998-6010.
Shastry B S, Trese M T. 2003. Overproduction and partial purification of the Norrie disease gene product, norrin, from a recombinant baculovirus. Biochem Biophys Res Commun 312: 229-234.
Smallwood P M, Williams J, Xu Q, Leahy D J, Nathans J. 2007. Mutational analysis of Norrin-Frizzled4 recognition. J Biol Chem 282: 4057-4068.
Smith P A, Tripp B C, DiBlasio-Smith E A, Lu Z, LaVallie E R, McCoy J M. 1998. A plasmid expression system for quantitative in vivo biotinylation of thioredoxin fusion proteins in Escherichia coli. Nucleic Acids Res 26: 1414-1420.
Sun P D, Davies D R. 1995. The cystine-knot growth-factor superfamily. Annu Rev Biophys Biomol Struct 24: 269-291.
Takada R, Satomi Y, Kurata T, Ueno N, Norioka S, Kondoh H, Takao T, Takada S. 2006. Monounsaturated fatty acid modification of Wnt protein: its role in Wnt secretion. Dev Cell 11: 791-801.
Tamai K, Semenov M, Kato Y, Spokony R, Liu C, Katsuyama Y, Hess F, Saint-Jeannet J P, He X. 2000. LDL-receptor-related proteins in Wnt signal transduction. Nature 407: 530-535.
Wang C, Wu H, Katritch V, Han G W, Huang X P, Liu W, Siu F Y, Roth B L, Cherezov V, Stevens R C. 2013. Structure of the human smoothened receptor bound to an antitumour agent. Nature.
Wang Y, Rattner A, Zhou Y, Williams J, Smallwood P M, Nathans J. 2012. Norrin/Frizzled4 signaling in retinal vascular development and blood brain barrier plasticity. Cell 151: 1332-1344.
Willert K, Brown J D, Danenberg E, Duncan A W, Weissman I L, Reya T, Yates J R, 3rd, Nusse R. 2003. Wnt proteins are lipid-modified and can act as stem cell growth factors. Nature 423: 448-452.
Xu Q, Wang Y, Dabdoub A, Smallwood P M, Williams J, Woods C, Kelley M W, Jiang L, Tasman W, Zhang K et al. 2004. Vascular development in the retina and inner ear: control by Norrin and Frizzled-4, a high-affinity ligand-receptor pair. Cell 116: 883-895.
Ye X, Wang Y, Nathans J. 2010. The Norrin/Frizzled4 signaling pathway in retinal vascular development and disease. Trends Mol Med 16: 417-425.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

1.-50. (canceled)

51. An isolated Norrin mutant polypeptide, the polypeptide comprising the amino acid sequence of SEQ ID NO: 1, said sequence having two or more amino acid substitutions at positions: 89, 123, 41, 43, 44, 45, 59, 60, 61, 120, 121, 122, 52, 53, 54, 107, 109, and 115 relative to SEQ ID NO: 1.

52. The isolated Norrin mutant polypeptide of claim 51, wherein the polypeptide has two or more amino acid substitutions at positions: 59, 122, 43, 45, 52, 53, 54, 93, 107, 109, 120, and 122.

53. The isolated Norrin mutant polypeptide of claim 51, wherein the polypeptide has two to seven amino acid substitutions relative to SEQ ID NO: 1.

54. The isolated Norrin mutant polypeptide of claim 51, wherein the polypeptide has two to five amino acid substitutions in SEQ ID NO: 1.

55. The isolated Norrin mutant polypeptide of claim 51, wherein the polypeptide has two or three amino acid substitutions in SEQ ID NO: 1.

56. The isolated Norrin mutant polypeptide of claim 51, wherein the polypeptide has two amino acid substitutions in SEQ ID NO: 1.

57. The isolated Norrin mutant polypeptide of claim 51, wherein the polypeptide comprises an amino acid sequence of SEQ ID NOs: 11-14, 18-20, 30-32 and 37.

58. The isolated Norrin mutant polypeptide of claim 57, wherein the polypeptide comprises an amino acid sequence of SEQ ID NO: 12.

59. The isolated Norrin mutant polypeptide of claim 51, wherein the polypeptide has an amino acid sequence having two or more amino acid substitutions as shown in Table 1.

60. The isolated Norrin mutant polypeptide of claim 51, having two or more amino acid substitutions: F89R, R41E, H43A, Y44A, V45A, M59A, V60A, L61A, Y120A, R121A, Y122A, L52A, Y53A, K54A, K54E, R107E, R109E, and R115E.

61. The isolated Norrin mutant polypeptide of claim 60, having two or more amino acid substitutions: H43A, V45A, K54E, R109E, Y120A, and Y122A.

62. An isolated Norrin mutant polypeptide, wherein the polypeptide has an amino acid sequence having one or more amino acid substitutions: F89R, H43A, Y44A, V45A, M59A, V60A, L61A, Y120A, R121A, Y122A, L52A, Y53A, K54A, and R107E.

63. The isolated Norrin mutant polypeptide of claim 62, having one or more amino acid substitutions: M59A, Y122A, L52A, Y53A, and R107E.

64. The isolated Norrin mutant polypeptide of claim 62, wherein the polypeptide has an amino acid sequence with a substitution H43A, V45A, L61A or Y122A.

65. The isolated Norrin mutant polypeptide of claim 51, wherein the amino acid substitution is a conservative amino acid substitution.

66. An isolated Norrin mutant polypeptide, the polypeptide comprising the amino acid sequence of SEQ ID NO: 1, said sequence having one to four amino acid substitutions at positions 52, 53, 59, and 122.

67. The isolated Norrin mutant polypeptide of claim 66, wherein the polypeptide has one to three amino acid substitutions relative to SEQ ID NO: 1.

68. The isolated Norrin mutant polypeptide of claim 66, wherein the polypeptide has one or two amino acid substitutions in SEQ ID NO: 1.

69. The isolated Norrin mutant polypeptide of claim 66, wherein the polypeptide has one or two amino acid substitutions: M59A, Y122A, L52A, or Y53A.

70. The isolated Norrin mutant polypeptide of claim 66, wherein the amino acid substitution is a conservative amino acid substitution.

71. The isolated Norrin mutant polypeptide of claim 51, wherein the first 24 amino acids are deleted.

72. A fusion protein comprising a Norrin mutant polypeptide of claim 51 fused to a maltose binding protein.

73. A composition comprising an isolated Norrin mutant polypeptide of claim 51.

74. A pharmaceutical composition comprising an isolated Norrin mutant polypeptide of claim 51 and a pharmaceutically acceptable carrier.

75. The pharmaceutical composition of claim 74, further comprising a secondary anti-angiogenic agent.

76. The pharmaceutical composition of claim 75, wherein the secondary anti-angiogenesis agent comprises an antagonist or inhibitor of VEGF, angiostatin, or endostatin.

77. A method of synthesizing a recombinant MBP-Norrin fusion protein, the method comprising:
providing a nucleic acid comprising a nucleic acid sequence encoding a bacterial maltose binding protein (MBP) operatively fused to a nucleic acid sequence encoding a Norrin construct;
expressing said nucleic acid in a bacterial strain comprising a gor and a trxB genetic mutation;
disrupting the integrity of the bacterial cell wall to provide a crude extract;
isolating the MBP-Norrin fusion protein from the crude extract using an amylose affinity column and
mixing the isolated MBP-Norrin fusion protein with a shuffling solution comprising arginine, reduced gluthathione, oxidized gluthathione, and a disulfide bond isomerase.

78. The method of to claim 77, wherein the nucleic acid encoding the Norrin construct is a Norrin wild-type protein or a Norrin mutant polypeptide.

79. The method of claim 77, wherein the nucleic acid encoding the MBP is fused to the N-terminus of the Norrin construct.

80. The method of claim 77, wherein the bacterial strain is an E. coli bacterial strain.

81. The method of claim 77, wherein the shuffling solution comprises an equimolar amount of reduced glutathione to oxidized glutathione.

82. The method of claim 77, further comprising a gel filtration purification step to isolate the MBP-Norrin fusion protein from the shuffling solution.

83. A method for reducing or inhibiting aberrant angiogenesis in a tissue, the method comprising: contacting the tissue exhibiting aberrant angiogenesis with a composition comprising the isolated Norrin mutant polypeptide of claim 51.

84. The method of claim 83, wherein the Norrin mutant polypeptide comprises an amino acid sequence of SEQ ID NO: 2-38 or 60-62.

85. The method of claim 83, wherein the Norrin mutant polypeptide comprises an amino acid sequence of claim 59.

86. The method of claim 83, wherein the Norrin mutant polypeptide comprises an amino acid sequence of SEQ ID NO: 2, 4, 15, 23, 26, 27, or 33.

87. A method for reducing or inhibiting aberrant angiogenesis in a tissue, the method comprising: contacting the tissue exhibiting aberrant angiogenesis with a composition comprising an isolated Norrin mutant polypeptide having one or more amino acid substitutions at positions 59, 122, 52, and 53 in SEQ ID NO: 1.

88. The method of claim 87, wherein the tissue is ocular tissue.

89. The method of claim 88, wherein the aberrant angiogenesis occurring in an ocular tissue is an ophthalmic disease selected from the group consisting of diabetic retinopathy, choroidal neovascularization, age-related macular degeneration, hypertensive retinopathy, retinopathy of prematurity, branched central retinal vein occlusion, central retinal vein occlusion, pathological myopia, diabetic macular edema, von Hippel-Lindau disease, histoplasmosis of the eye, subconjectival hemorrhage and corneal neovascularization.

90. The method of claim 87, wherein the tissue is a cancerous tissue.

91. The method of claim 90, wherein the cancerous tissue is a cancer in a subject.

92. The method of claim 91, wherein the cancer is malignant melanoma, malignant glioma, pancreas carcinoma, colorectal carcinoma, non-small cell lung cancer, prostate carcinoma, breast cancer, hematological malignancies, hepatocellular carcinoma, sarcoma, renal cell carcinoma, melanoma, cutaneous squamous cell carcinoma, endometrial cancer, esophageal carcinoma and cervical cancer.

93. The method of claim 90, wherein the cancerous tissue comprises cells expressing a higher level of Norrin mediated angiogenesis as compared to noncancerous cells of comparable tissue type.

94. The method of claim 77 further comprising the step, removing the N-terminal maltose binding protein from the recombinant MBP-Norrin fusion protein.

95. An isolated MBP-Norrin fusion protein, the fusion protein comprising an amino acid sequence of SEQ ID NO: 40.

96. A polynucleotide encoding the fusion protein of claim 95 comprising the nucleotide sequence of SEQ ID NO: 47.

97. A vector comprising the polynucleotide of claim 96.

98. A host cell comprising the vector of claim 97.

99. A fusion protein comprising a Norrin mutant polypeptide of claim 62, fused to a maltose binding protein.

100. A composition comprising an isolated Norrin mutant polypeptide of claim 62.

101. A pharmaceutical composition comprising an isolated Norrin mutant polypeptide of claim 62, and a pharmaceutically acceptable carrier.

102. The pharmaceutical composition of claim 101, further comprising a secondary anti-angiogenic agent.

103. The pharmaceutical composition of claim 102, wherein the secondary anti-angiogenesis agent comprises an antagonist or inhibitor of VEGF, angiostatin, or endostatin.
